SEQUENCE LISTING (68 sequences)

SEQ ID NO: 1 - DNA, 858 bp, Mus musculus
tttcagtggctgctactcggcgcttcagtcgcggtcgcttcagtcgtcagcatggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgtgatcaagcatcatgatgctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaac

SEQ ID NO: 2 - protein, 119 aa, Mus musculus
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDM

SEQ ID NO: 3 - DNA, 1675 bp, Homo sapiens
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 4 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 5 - DNA, 1736 bp, Mus musculus
tcagacacccgggatcccagatgggggcgatggctccgcgcacgctgctcctgctgctggcggccgccctggccccgactcagacccgcgcgggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctactacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgatcgttgctgttctgggtgtccttggagctatggccatcattggagctgtggtggcttttgtgatgaagagaaggagaaacacaggtggaaaaggaggggactatgctctggctccaggctcccagagctctgaaatgtctctccgagattgtaaagcgtgaagacagctgcctggactgtactgagtgacagacgatgtgttcaggtctctcctgtgacatccagagccctcagttctctttacacaacattgtctgatgttccctgtgagcttgggttcagtgtgaagaactgtggagcccagcctgccctgcacaccaggaccctatccctgcactgccctgtgttcccttccatagccaaccttgctgctccagccaaacactgggggacatctgcatcctgtaagctccatgctaccctgagctgcagctcctcacttccacactgagaataataatttgaatgtgggtggctggagagatggctcagcgctgactgctcttccaaaggtcctgagttcaaatcccagcaaccacatggtggctcacaaccatctgtaatgggatctaacaccctcttctgcagtgtctgaagacagctacagtgtacttacatataataataaataagtctttaaaaaataatttgaaagtgaccttgattgttaacatcttgatctagggctgatttcttgttaatttcatggattgagaatgcttagaggtttttttgtttgtttgattgatttttttgaagaaataaatggcagatgaaggaacttccag

SEQ ID NO: 6 - protein, 362 aa, Mus musculus
MGAMAPRTLLLLLAAALAPTQTRAGPHSMRYFETAVSRPGLEEPRYISVGYVDNKEFVRFDSDAENPRYEPRAPWMEQEGPEYWERETQKAKGQEQWFRVSLRNLLGYYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 7 - DNA, 1611 bp, Homo sapiens
gagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaattaatggaacgcatgagaaccttccagagtcca

SEQ ID NO: 8 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 9 - DNA, 2109 bp, Artificial Sequence
  The mRNA sequence of the engineered mouse B2M-HLA-A2.1 after humanization
tttcagtggctgctactcggcgcttcagtcgcggtcgcttcagtcgtcagcatggccgtcatggcgccccgaaccctcgtcctgctactctcgggggctctggccctgacccagacctgggcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggtggcggatccggcggaggcggctcgggtggcggcggctctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgatcgttgctgttctgggtgtccttggagctatggccatcattggagctgtggtggcttttgtgatgaagagaaggagaaacacaggtggaaaaggaggggactatgctctggctccaggctcccagagctctgaaatgtctctccgagattgtaaagcgtgaagacagctgcctggactgtactgagtgacagacgatgtgttcaggtctctcctgtgacatccagagccctcagttctctttacacaacattgtctgatgttccctgtgagcttgggttcagtgtgaagaactgtggagcccagcctgccctgcacaccaggaccctatccctgcactgccctgtgttcccttccatagccaaccttgctgctccagccaaacactgggggacatctgcatcctgtaagctccatgctaccctgagctgcagctcctcacttccacactgagaataataatttgaatgtgggtggctggagagatggctcagcgctgactgctcttccaaaggtcctgagttcaaatcccagcaaccacatggtggctcacaaccatctgtaatgggatctaacaccctcttctgcagtgtctgaagacagctacagtgtacttacatataataataaataagtctttaaaaaataatttgaaagtgaccttgattgttaacatcttgatctagggctgatttcttgttaatttcatggattgagaatgcttagaggtttttttgtttgtttgattgatttttttgaagaaataaatggcagatgaaggaacttccag

SEQ ID NO: 10 - DNA, 4694 bp, Artificial Sequence
  The DNA sequence of the engineered mouse B2M-HLA-A2.1 after humanization
tttcagtggctgctactcggcgcttcagtcgcggtcgcttcagtcgtcagcatggccgtcatggcgccccgaaccctcgtcctgctactctcgggggctctggccctgacccagacctgggcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggtggcggatccggcggaggcggctcgggtggcggcggctctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctggatcctgtgtgacatacctgtaccttgtcctccagagtcaggggctgggagtcattttctctggctacagactttgtgatggctgttcactcggactgacagttaacgttggtcagcaagatgaccacagtggttgagtctcagtggtgggacccttccagtagcatatgcccctaattttgatatgaactcaaacagatattaaattacttattttccattccctattccattctgtgactatctctctcatgctattgaacatcacataaggatggccatgttcacccactggctcatgtggattccctcttagcttctttgtcccaaaagaaaatgtgcagtcctgtgctgaggggaccagctctgcttttggtcactagtgcaatgacagtgtagtgtcaaatagacacatagttcactctcatcattgatttaactgagtcttgtgtagatttcagtttgtcttgttaattgtggaatttcttaaatcttccacacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatggggtaaggagggtgtgggtgcagagctggggtcagggaaagctggagccttctgcagaccctgagctggtcagggatgagagctggggtcataaccctcaccttcatttcctgtacctgtccttcccagagcctcctccgtccactgactcttacatggtgatcgttgctgttctgggtgtccttggagctatggccatcattggagctgtggtggcttttgtgatgaagagaaggagaaacacaggtaagaaagggcagggtctgagttttctctcagcctcctttagaagtgtgctctgctcattaatggggaacacagccacaccccacattgctactgtctctaactgggtctgctgtcagttctgggaatttccagtgtcaagatcttccttgaactctcacagcttttcttttcacaggtggaaaaggaggggactatgctctggctccaggttagtgtggggacaggattgttctgggggacattggagtgaagttggagatgatgggagctctgggaatccataatagctcctccagagaaatcttctaggggcctgagttgtgccatgaagtgaatacattcatgtacatatgcatatacatttgttttgttttaccctaggctcccagagctctgaaatgtctctccgagattgtaaaggtgacactctagggtctgattggggaggggcaatgtggacatgattgggtttcagggactcccagaatctcctgagagtgagtggtgggttgctggaatgttgtcttcacagtgatggttcatgactctcattctctagcgtgaagacagctgcctggactgtactgagtgacagacgatgtgttcaggtctctcctgtgacatccagagccctcagttctctttacacaacattgtctgatgttccctgtgagcttgggttcagtgtgaagaactgtggagcccagcctgccctgcacaccaggaccctatccctgcactgccctgtgttcccttccatagccaaccttgctgctccagccaaacactgggggacatctgcatcctgtaagctccatgctaccctgagctgcagctcctcacttccacactgagaataataatttgaatgtgggtggctggagagatggctcagcgctgactgctcttccaaaggtcctgagttcaaatcccagcaaccacatggtggctcacaaccatctgtaatgggatctaacaccctcttctgcagtgtctgaagacagctacagtgtacttacatataataataaataagtctttaaaaaataatttgaaagtgaccttgattgttaacatcttgatctagggctgatttcttgttaatttcatggattgagaatgcttagaggtttttttgtttgtttgattgatttttttgaagaaataaatggcagatgaaggaacttccagaatctgggtcactatgctgtgtgtatctgttgggacaggatgaggctgtggcagctgagtgtgaacagggctgtgcccaggtgggctcagtccattttgatctgtgatggggccacacctcggctgtgtcacctctgggctctgttctctccatcactctgaggcacatcctgagagtttgtgatcacaaagacacagggagggcctgagccttgccctgtccccaggattatgagcccctagggctaaagatcagagactcaactaggacagagctctgcgggtacatcttagccctttcagtctctagttggtaggcactagatttaccttctggaggcttccggacactcagggaaagaaatggacagggttgtaacttcatgtaaggcaccgtcactgatgtgtcagaaggaagttgaggagcgtgagagggaacgtgggtgtctctgtcaggtggagtctagtggtagaaaatccagcttttcggtgaaatccagggcccttgaggccaaaagctcactcaaatcttgtattatatttatttctaggaaaggggaattgatgaagggggtggggatgggtgatctgcccagacaagcagttaccaaatggtggagccagaactcacctcctttaagaaggtgattgtcctacaaactcatttgatggtgaggtctggaatgtaaataatgtaatgttcggctaaccttctccttctctccctcccttccccttttcttttcagctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaac

SEQ ID NO: 11 - DNA, 1409 bp, Artificial Sequence
  5′ arm
gcatttcctagtacagttcaacacagtgtttagtataatttaatattaacatttcaggtctttaatgtattaaatgagtttttctttttaattagatgaagtatcttaactcacacctttatacataaaaattattcttatatttcagttacaaaaagggatagcaaaataacataaatgattatgattcttaaacattactggatcaaaatactatatgctgggtagacactgtaggattgggtctctgtttattatctcattaatgtccgtggagactattttatttttttctagtttagacacagcctccacactagagattgttaaaggtttttaaaaatatgttcaagaaatccagaagttatgttgttctccttggggaagccaaggccaaagccacttcaactgcaagcatggccatttctccatgtcctgatgccccatgccctcttcctacccttcctcactcgagctgcttgcggtggtgtctccctgtgctcacagcctgcctctgtcctggcttttggccaggggtttaacttctctactgggtccaccgctgatgtaatatacattctgagtaaaactatggcagcctttgtcttccgagccgataagcagaaaccccaagggaatgcatctcaaggtttatctcctcatccagaaaaaataatttattcggggattcacttcacttggtcacttagtttaccaatggccctgttctaagaaagtactactaagagaatggacaggaaacccagctaagccattgtccttccccaaaacccatggacagaacaaagaaaattacctaaatttttcaagggtacccgattaagttctccatccaaagctctaaaaggagaaacctgctagaagcaaggtcagaaatcctctcagttgcctttaggacaggagggtctttttacacacggaatcctgaaactgcctttgtatttctgggctgagattataaactaagggttgagttctgccagttaatgctcttaattgtcctggctttagttttcaagaatgcaaacttcaggtcctaagtccttttctgagtgggatattgtcagcaattgaataaatgaaggcggtcccaggctgaacgaccagatacaccaaactcaagagcacaccctagatagtagggcaccaagggtccagcccaggctgtttgaaatatcacgggactttataagaacatgaaactgaaaatgggaaagtccctttgtaacctagttcagcatcaacagctaggagactggtgacgacctccggatctgagtccggattggctgtgagttcaggaactatataagagcgcgcgccctggctggctctcatttcagtggctgctactcggcgcttcagtcgcggtcgcttcagtcgtcagc

SEQ ID NO: 12 - DNA, 1343 bp, Artificial Sequence
  3′ arm
gacagagctctgcgggtacatcttagccctttcagtctctagttggtaggcactagatttaccttctggaggcttccggacactcagggaaagaaatggacagggttgtaacttcatgtaaggcaccgtcactgatgtgtcagaaggaagttgaggagcgtgagagggaacgtgggtgtctctgtcaggtggagtctagtggtagaaaatccagcttttcggtgaaatccagggcccttgaggccaaaagctcactcaaatcttgtattatatttatttctaggaaaggggaattgatgaagggggtggggatgggtgatctgcccagacaagcagttaccaaatggtggagccagaactcacctcctttaagaaggtgattgtcctacaaactcatttgatggtgaggtctggaatgtaaataatgtaatgttcggctaaccttctccttctctccctcccttccccttttcttttcagctctgaagattcatttgaacctgcttaattacaaatccagtttctaatatgctatacaatttatgcacgcagaaagaaatagcaatgtacacatcaccttctttatatcttactttaaatattttatgcatgttttcaaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaactaaaaaaaaaaaaaaaaattgttgccagacattgccagtgttacatgaagtggtttctaggtagcataaatgcctgcaagcacgtgcgtatttttcatcatctaaagaggcagtgggtatcttagaaggttcttgcagaagcaggctcctcaactaagggtcgagaggaaagtgactaggaaaggcctccaaagcagagcagtaggaggtggtgatgaaggtcagggaataataggaagaaactgagaggggagtgaggatctcagacgtgctggtcccacagggagctctagagcagagatcacatcttacagtttgtcttggtgatgtgcacagcttgttgctggctccacaggactgtatgctaggatgaaagcccagatactttggggtgcaggttatggttatggggtgcaagttatggttattgg

SEQ ID NO: 13 - DNA, 72 bp, Homo sapiens
atggccgtcatggcgccccgaaccctcgtcctgctactctcgggggctctggccctgacccagacctgggcg

SEQ ID NO: 14 - DNA, 297 bp, Homo sapiens
atccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 15 - DNA, 831 bp, Homo sapiens
ggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatct

SEQ ID NO: 16 - DNA, 2486 bp, Mus musculus
ggatcctgtgtgacatacctgtaccttgtcctccagagtcaggggctgggagtcattttctctggctacagactttgtgatggctgttcactcggactgacagttaacgttggtcagcaagatgaccacagtggttgagtctcagtggtgggacccttccagtagcatatgcccctaattttgatatgaactcaaacagatattaaattacttattttccattccctattccattctgtgactatctctctcatgctattgaacatcacataaggatggccatgttcacccactggctcatgtggattccctcttagcttctttgtcccaaaagaaaatgtgcagtcctgtgctgaggggaccagctctgcttttggtcactagtgcaatgacagtgtagtgtcaaatagacacatagttcactctcatcattgatttaactgagtcttgtgtagatttcagtttgtcttgttaattgtggaatttcttaaatcttccacacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatggggtaaggagggtgtgggtgcagagctggggtcagggaaagctggagccttctgcagaccctgagctggtcagggatgagagctggggtcataaccctcaccttcatttcctgtacctgtccttcccagagcctcctccgtccactgactcttacatggtgatcgttgctgttctgggtgtccttggagctatggccatcattggagctgtggtggcttttgtgatgaagagaaggagaaacacaggtaagaaagggcagggtctgagttttctctcagcctcctttagaagtgtgctctgctcattaatggggaacacagccacaccccacattgctactgtctctaactgggtctgctgtcagttctgggaatttccagtgtcaagatcttccttgaactctcacagcttttcttttcacaggtggaaaaggaggggactatgctctggctccaggttagtgtggggacaggattgttctgggggacattggagtgaagttggagatgatgggagctctgggaatccataatagctcctccagagaaatcttctaggggcctgagttgtgccatgaagtgaatacattcatgtacatatgcatatacatttgttttgttttaccctaggctcccagagctctgaaatgtctctccgagattgtaaaggtgacactctagggtctgattggggaggggcaatgtggacatgattgggtttcagggactcccagaatctcctgagagtgagtggtgggttgctggaatgttgtcttcacagtgatggttcatgactctcattctctagcgtgaagacagctgcctggactgtactgagtgacagacgatgtgttcaggtctctcctgtgacatccagagccctcagttctctttacacaacattgtctgatgttccctgtgagcttgggttcagtgtgaagaactgtggagcccagcctgccctgcacaccaggaccctatccctgcactgccctgtgttcccttccatagccaaccttgctgctccagccaaacactgggggacatctgcatcctgtaagctccatgctaccctgagctgcagctcctcacttccacactgagaataataatttgaatgtgggtggctggagagatggctcagcgctgactgctcttccaaaggtcctgagttcaaatcccagcaaccacatggtggctcacaaccatctgtaatgggatctaacaccctcttctgcagtgtctgaagacagctacagtgtacttacatataataataaataagtctttaaaaaataatttgaaagtgaccttgattgttaacatcttgatctagggctgatttcttgttaatttcatggattgagaatgcttagaggtttttttgtttgtttgattgatttttttgaagaaataaatggcagatgaaggaacttccagaatctgggtcactatgctgtgtgtatctgttgggacaggatgaggctgtggcagctgagtgtgaacagggctgtgcccaggtgggctcagtccattttgatctgtgatggggccacacctcggctgtgtcacctctgggctctgttctctccatcactctgaggcacatcctgagagtttgtgatcacaaagacacagggagggcctgagccttgccctgtccccaggattatgagcccctagggctaaagatcagagactcaactag

SEQ ID NO: 17 - DNA, 23 bp, Artificial Sequence
  sgRNA1 targeting site sequence
cctggccaatcccgtcgggaagg

SEQ ID NO: 18 - DNA, 23 bp, Artificial Sequence
  sgRNA2 targeting site sequence
ccgtcagcacactcgcaaacagg

SEQ ID NO: 19 - DNA, 23 bp, Artificial Sequence
  sgRNA3 targeting site sequence
gttctccttcccgacgggattgg

SEQ ID NO: 20 - DNA, 23 bp, Artificial Sequence
  sgRNA4 targeting site sequence
actctggatagcatacaggccgg

SEQ ID NO: 21 - DNA, 23 bp, Artificial Sequence
  sgRNA5 targeting site sequence
ctggtgcttgtctcactgaccgg

SEQ ID NO: 22 - DNA, 23 bp, Artificial Sequence
  sgRNA6 targeting site sequence
ggggaaagaggcactcactctgg

SEQ ID NO: 23 - DNA, 23 bp, Artificial Sequence
  sgRNA7 targeting site sequence
gacaagcaccagaaagaccaggg

SEQ ID NO: 24 - DNA, 23 bp, Artificial Sequence
  sgRNA8 targeting site sequence
ctggaggcttccggacactcagg

SEQ ID NO: 25 - DNA, 23 bp, Artificial Sequence
  sgRNA9 targeting site sequence
tgatcaagcatcatgatggtagg

SEQ ID NO: 26 - DNA, 23 bp, Artificial Sequence
  sgRNA10 targeting site sequence
aggagcgtgagagggaacgtggg

SEQ ID NO: 27 - DNA, 23 bp, Artificial Sequence
  sgRNA11 targeting site sequence
gaggaacgtagccatgtcactgg

SEQ ID NO: 28 - DNA, 23 bp, Artificial Sequence
  sgRNA12 targeting site sequence
catgtcactggccctctaaaggg

SEQ ID NO: 29 - DNA, 23 bp, Artificial Sequence
  sgRNA13 targeting site sequence
catgtgatcaagcatcatgatgg

SEQ ID NO: 30 - DNA, 23 bp, Artificial Sequence
  sgRNA14 targeting site sequence
acccgcagagctctgtcactcgg

SEQ ID NO: 31 - DNA, 23 bp, Artificial Sequence
  sgRNA15 targeting site sequence
ctctgtcactcggctcctctggg

SEQ ID NO: 32 - DNA, 20 bp, Artificial Sequence
  sgRNA-3 upstream sequence
gttctccttcccgacgggat

SEQ ID NO: 33 - DNA, 23 bp, Artificial Sequence
  sgRNA-3 forward oligonucleotide
taggttctccttcccgacgggat

SEQ ID NO: 34 - DNA, 19 bp, Artificial Sequence
  sgRNA-3 downstream sequence
atcccgtcgggaaggagaa

SEQ ID NO: 35 - DNA, 23 bp, Artificial Sequence
  sgRNA-3 reverse oligonucleotide
aaacatcccgtcgggaaggagaa

SEQ ID NO: 36 - DNA, 20 bp, Artificial Sequence
  sgRNA-14 upstream sequence
acccgcagagctctgtcact

SEQ ID NO: 37 - DNA, 24 bp, Artificial Sequence
  sgRNA-14 forward oligonucleotide
taggacccgcagagctctgtcact

SEQ ID NO: 38 - DNA, 19 bp, Artificial Sequence
  sgRNA-14 downstream sequence
agtgacagactctgcgggt

SEQ ID NO: 39 - DNA, 23 bp, Artificial Sequence
  sgRNA-14 reverse oligonucleotide
aaacagtgacagactctgcgggt

SEQ ID NO: 40 - DNA, 132 bp, Artificial Sequence
  DNA fragment containing the T7 promoter and sgRNA fragment
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 41 - DNA, 25 bp, Artificial Sequence
  5′end primer
gaatgtgtgcctcctctcagtttcc

SEQ ID NO: 42 - DNA, 25 bp, Artificial Sequence
  5′end primer
tccttcccgttctccaggtatctgc

SEQ ID NO: 43 - DNA, 23 bp, Artificial Sequence
  3′end primer
gcggctactacaaccagagcgag

SEQ ID NO: 44 - DNA, 27 bp, Artificial Sequence
  3′end primer
tccagcaataagaaccagtccctagct

SEQ ID NO: 45 - DNA, 25 bp, Artificial Sequence
  Probe primer
atgaggtctttttgtgggcagagca

SEQ ID NO: 46 - DNA, 25 bp, Artificial Sequence
  Probe primer
ctccctacggccacatcaccattac

SEQ ID NO: 47 - DNA, 24 bp, Artificial Sequence
  Probe primer
taacttcatgtaaggcaccgtcac

SEQ ID NO: 48 - DNA, 23 bp, Artificial Sequence
  Probe primer
tccagacctcaccatcaaatgag

SEQ ID NO: 49 - DNA, 25 bp, Artificial Sequence
  5′end primer for KO mouse
gaataaatgaaggcggtcccaggct

SEQ ID NO: 50 - DNA, 25 bp, Artificial Sequence
  3′end primer for KO mouse
aggtgagttctggctccaccatttg

SEQ ID NO: 51 - DNA, 45 bp, Artificial Sequence
  The flexible linker nucleotide sequence
ggaggtggcggatccggcggaggcggctcgggtggcggcggctct

SEQ ID NO: 52 - DNA, 1891 bp, Artificial Sequence
  mRNA sequence of humanized B-NDG mouse
tttcagtggctgctactcggcgcttcagtcgcggtcgcttcagtcgtcagcatggccgtcatggcgccccgaaccctcgtcctgctactctcgggggctctggccctgacccagacctgggcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggtggcggatccggcggaggcggctcgggtggcggcggctctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaa

SEQ ID NO: 53 - DNA, 1342 bp, Artificial Sequence
  3′ arm
gacagagctctgcgggtacatcttagccctttcagtctctagttggtaggcactagatttaccttctggaggcttccggacactcagagaaagaaatggacagggttgtaacttcatgtaaggcaccgtcactgatgtgtcagaaggaagttgaggagcgtgagagggaacgtgggtgtctctgtcaggtggagtctagtggtagaaaatccagcttttcagtgaaatccagggcccttgaggccaaaagctcactcaaatcttgtattatatttatttctaggaaaggggaattgatgaagggggtggggatgggtgatctgcccagacaagcagttaccaaatggtggagccagaactcacctcctttaagaaggtgattgtcctacaaactcatttgatggtgaggtctggaatgtaaataatgtaatgttcggctaaccttctccttctctccctcccttccccttttcttttcagctctgaagattcatttgaacctgcttaattacaaatccagtttctaatatgctatacaatttatgcacgcagaaagaaatagcaatgtacacatcaccttctttatatcttactttaaatgttttatgcatgttttcaaaaattggaaatatcctagatagctgagcaataaatcttcaataagtattttgatcagaataataaatataattttaagaacaatagttgatcatatgccaaaccctctgtacttctcattacttggatgcagttactcatctttggtctatcacaacataagtgacatactttccttttggtaaagcaaagaggcctaattgaagtctgtcactgtgcccaatgcttagcaattctcacccccaaccctgtggctacttctgcttttgttacttttactaaaaataaaaaactaaaaaaaaaaaaaaaaattgctgccagacattgccagtgttacatgaagtggtttctaggtagcctaaatgcctgcaagcacgtgcgtatttttcatcatctaaagaggcagtgggtatcttagaaggttcttgcagaagcaggctcctcaactaagggtcgagaggaaagtgactaggaaaggcctccaaagcagagcagtaggaggtggtgatgaaggtcagggaataataggaagaaactgagaggggagtgaggatctcagacgtgctggtcccacagggagctctagagcagagatcacatcttacagtttgtcttggtgatgtgcacagcttgttgctggctccacaggactgtatgctaggatgaaagcccagatactttggggtgcaggttatggttatggggtgcaagttatggttattgg

SEQ ID NO: 54 - DNA, 3944 bp, Homo sapiens
ggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttggcccttcagtagaaccttgtcccactaagacctgtgatcacagggagttggatgtcacctagggtggtccctgcatacaaatctccttgcggtatcaagagacaaattttcagacctgtctaggtcttgccttcctcccagggctttttcctcaattgtattttcaatttttctccaatctttttaaaggaaccagattgtgacatttgcagagaggagtggtcccatagtttctcatcatgattaactttctgttggaactcctgttctgccctcctactcttcttcctgctctgaattgtagtaatcctagtgctggctccaatccaaactcatagatttataaagcagagtctaatttagattcatatgtggttggaaaattgtacccataaggctagggttattgttcctgaagagaaatatatggttttgtgctgaagtgtgcaggagggttggtgtgggaggagggaggacacacaagcagccctggtgagaaaagcactggcggcgtggatgtccatgtgaacttatgttctttagctgccacaaaacagcatttgccctgtggctacattaataaaggtatgggctttagaatagggagatgctctacagtggtcattcattcaactgacatttgttgtctgctagggatatgactgctttt

SEQ ID NO: 55 - DNA, 24 bp, Artificial Sequence
  5′ end primer
cagctccaaagagaaccaggccag

SEQ ID NO: 56 - DNA, 24 bp, Artificial Sequence
  3′ end primer
taccctgcggagatcacactgacc

SEQ ID NO: 57 - DNA, 24 bp, Artificial Sequence
  Probe primer
ttctgatgctccttccttccgtgc

SEQ ID NO: 58 - DNA, 24 bp, Artificial Sequence
  Probe primer
ttctctgtgctcagtgttccctgc

SEQ ID NO: 59 - protein, 362 aa, Homo sapiens
MAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 60 - DNA, 1409 bp, Artificial Sequence
  5′ arm
gcatttcctagtacagttcaacacagtgtttagtataatttaatattaacatttcaggtctttaatgtattaaatgagtttttctttttaattagatgaagtatcttaactcacacctttatacataaaaattattcttatatttcagttacaaaaagggatagcaaaataacataaatgattatgattcttaaacattactggatcaaaatactatatgctgggtagacactgtaggattgggtctctgtttattatctcattaatgtccgtggagactattttatttttttctagtttagacacagcctccacactagagattgttaaaggtttttaaaaatatgttcaagaaatccagaagttatgttgttctccttggggaagccaaggccaaagccacttcaactgcaagcatggccatttctccatgtcctgatgccccatgccctcttcctacccttcctcactcgagctgcttgcggtggtgtctccctgtgctcacagcctgcctctgtcctggcttttggccaggggtttaacttctctactgggtccaccgctgatgtaatatacattctgagtaaaactatggcagcctttgtcttccgagccgataagcagaaaccccaagggaatgcatctcaaggtttatctcctcatccagaaaaaataatttattcgggtattcacttcacttggtcacttagtttaccaatggccctgttctaagaaagtactactaagagaatggacaggaaacccagctaagccattgttcttccccaaaacccatggacagaacaaagaaaattacctaaatttttcaagggtacccgattaagttctccatccaaagctctaaaaggagaaacctgctagaagcaaggtcagaaatcctctcagttgcctttaggacaggagggtctttttacacacggaatcctgaaactgcctttgtatttctgggctgagattataaactaagggttgagttctgccagttaatgctcttaattgtcctggctttagttttcaagaatgcaaacttcaggtcctaagtccttttctgagtgggatattgtcagcaattgaataaatgaaggcggtcccaggctgaacgaccagatacaccaaactcaagagcacaccctagatagtagggcaccaagggtccagcccaggctgtttgaaatatcacgggactttataagaacatgaaactgaaaatgggaaagtccctttgtaacctagttcagcattaacagctaggagactggtgacgacctccggatctgagtccggattggctgtgagttcaggaactatataagagcgcgcgccctggctggctctcatttcagtggctgctactcggcgcttcagtcgcggtcgcttcagtcgtcagc

SEQ ID NO: 61 - protein, 476 aa, Artificial Sequence
  Protein sequence of humanized C57
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 62 - protein, 479 aa, Artificial Sequence
  Protein sequence of humanized NDG
MAVMAPRTLVLLLSGALALTQTWAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 63 - protein, 337 aa, Artificial Sequence
  Human B2M and HLA-A2.1 protein signal peptide, Alpha-1and Alpha-2 regions
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSMAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRT

SEQ ID NO: 64 - protein, 358 aa, Artificial Sequence
  The signal peptide, Alpha-1 and Alpha-2 regions of human HLA-A2.1 protein and Alpha-3, connecting peptide, transmembrane region and intracellular region of mouse H2-D1
MAPRTLVLLLSGALALTQTWAGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKA

SEQ ID NO: 65 - DNA, 3690 bp, Artificial Sequence
  the BNDG-A fragment
atggccgtcatggcgccccgaaccctcgtcctgctactctcgggggctctggccctgacccagacctgggcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggtggcggatccggcggaggcggctcgggtggcggcggctctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttggc

SEQ ID NO: 66 - protein, 119 aa, Rattus norvegicus
MARSVTVIFLVLVSLAVVLAIQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 67 - protein, 15 aa, Artificial Sequence
  The linker polypeptide sequence
GGGGSGGGGSGGGGS

SEQ ID NO: 68 - protein, 5 aa, Artificial Sequence
  The linker polypeptide sequence
GGGGS

(19) United States
(12) Patent Application Publication
(10) Pub. No.: US 2023/0072216 A1
(43) Pub. Date: Mar. 9, 2023

(54) GENETICALLY MODIFIED NON-HUMAN ANIMAL WITH HUMAN OR CHIMERIC MHC PROTEIN COMPLEX

(71) Applicant: Biocytogen Pharmaceuticals (Beijing) Co., Ltd., Beijing (CN)

(72) Inventors: Yuelei Shen, Beijing (CN); Chaoshe Guo, Beijing (CN); Meiling Zhang, Beijing (CN); Rui Huang, Beijing (CN); Yanan Guo, Beijing (CN); Yang Bai, Beijing (CN); Jiawei Yao, Beijing (CN); Chengzhang Shang, Beijing (CN)

(21) Appl. No.: 17/791,026

(22) PCT Filed: Jan. 8, 2021
(86) PCT No.: PCT/CN2021/070967

§ 371 Date: Jul. 6, 2022

(30) Foreign Application Priority Data
Jan. 10, 2020 (CN) 202010028231.9

(51) Int. Cl. A01K 67/027; C07K 14/74; C12N 15/85

(52) U.S. Cl. CPC A01K 67/0275; C07K 14/70539; C12N 15/85; A01K 2217/072; A01K 2217/075; A01K 2227/105; A01K 2267/03; C07K 2319/01

(57) ABSTRACT

The present disclosure relates to genetically modified non-human animals that express a human or chimeric (e.g., humanized) major histocompatibility complex (MHC) protein complex, and methods of use thereof.

### TECHNICAL FIELD

This disclosure relates to genetically modified animals which express a human or chimeric (e.g., humanized) major histocompatibility complex (MHC) protein complex, and methods of use thereof.

### BACKGROUND

Major histocompatibility complex (MHC) class I and class II proteins play a pivotal role in the adaptive branch of the immune system. Both classes of proteins share the task of presenting peptides on the cell surface for recognition by T cells. Immunogenic peptide-MHC class I (pMHCI) complexes are presented on nucleated cells and are recognized by cytotoxic CD8+ T cells. In contrast, the presentation of pMHCII by antigen-presenting cells (e.g., dendritic cells (DCs), macrophages, or B cells) can activate CD4+ T cells, leading to the coordination and regulation of effector cells.

Human MHC protein complexes are highly polymorphic, and are different from animals' MHC. This difference may result in a higher rate of failure in drug development. Particularly, the test results obtained from the use of conventional experimental animals for in vivo pharmacological test may not reflect the real disease state and the cellular interaction in human, thus the results in many clinical trials can be significantly different from the animal experimental results. There is a need for genetically engineered animals that can generate an immune system that is more similar to the human immune system.

### SUMMARY

This disclosure is related to genetically-modified animals that express a human or chimeric (e.g., humanized) major histocompatibility complex (MHC) protein complex. In some embodiments, the animal is used as an immune-deficient animal model (e.g., having a CD132 gene knockout). As MHC is involved in T cell development, the human or humanized MHC can provide a better environment for T cell development in an animal model with the reconstructed human immune system.

In one aspect, the disclosure is related to a genetically-modified non-human animal expressing a fusion protein comprising β2 microglobulin (B2M) and a human or humanized major histocompatibility complex (MHC) molecule (e.g., MHC α chain).

In some embodiments, the genome of the animal comprises at least one chromosome comprising a sequence encoding the fusion protein.

In some embodiments, the fusion protein comprises a human or humanized B2M protein.

In some embodiments, the MHC molecule is a MHC class I or MHC class II α chain.

In some embodiments, the MHC α chain is a human HLA-A protein.

In some embodiments, the MHC α chain is a chimeric MHC α chain. In some embodiments, the MHC α chain is a human HLA-A/mouse H2-D1 chimeric molecule.

In some embodiments, the fusion protein comprises a human B2M protein and a chimeric MHC α chain comprising human HLA-A α1 and/or α2 domains. In some embodiments, the chimeric MHC α chain further comprises a mouse H2-D1 domain (e.g., α1, α2 and/or α3 domains).

In some embodiments, the fusion protein comprises a human B2M protein and a human HLA-A protein.

In some embodiments, the sequence encoding the fusion protein is operably linked to an endogenous regulatory element (e.g., a promoter) at the endogenous β2 microglobulin (B2M) gene locus in the at least one chromosome.

In some embodiments, the sequence encoding the fusion protein is operably linked to an endogenous regulatory element (e.g., a promoter) at the endogenous MHC gene locus in the at least one chromosome.

In some embodiments, the animal is a mouse, and the sequence encoding the MHC molecule is operably linked to an endogenous regulatory element at the mouse H2-D1 gene locus in the at least one chromosome.

In some embodiments, the human HLA-A is human HLA-A2.1. In some embodiments, the human HLA-A is human HLA-A1*0101.

In some embodiments, the fusion protein comprises (a) a human B2M; and (b) a human HLA-A.

In some embodiments, the human B2M and the human HLA-A are linked via a linker peptide sequence.

In some embodiments, the human B2M comprises or consists of an amino acid sequence that is at least 70%, 75%, 80%, 85%, 90%, 95%, 99%, or 100% identical to SEQ ID NO: 4 or amino acids 21-119 of SEQ ID NO: 4.

In some embodiments, the human HLA-A is HLA-A2.1 or HLA-A1*0101.

In some embodiments, the human HLA-A comprises or consists of an amino acid sequence that is at least 70%, 75%, 80%, 85%, 90%, 95%, 99%, or 100% identical to SEQ ID NO: 8, amino acids 25-365 of SEQ ID NO: 8, SEQ ID NO: 59, or amino acids 22-362 of SEQ ID NO: 59.

In some embodiments, the fusion protein comprises or consists of an amino acid sequence that is at least 70%, 75%, 80%, 85%, 90%, 95%, 99%, or 100% identical to SEQ ID NO: 62.

In some embodiments, the fusion protein comprises (a) a human B2M; and (b) a chimeric MHC α chain.

In some embodiments, the human B2M and the chimeric MHC α chain are linked via a linker peptide sequence.

In some embodiments, the human B2M comprises or consists of an amino acid sequence that is at least 70%, 75%, 80%, 85%, 90%, 95%, 99%, or 100% identical to SEQ ID NO: 4 or amino acids 21-119 of SEQ ID NO: 4.

In some embodiments, the chimeric MHC α chain comprises human HLA-A α1 and α2 domains.

In some embodiments, the chimeric MHC α chain further comprises a human HLA-A α3 domain.

In some embodiments, the chimeric MHC α chain further comprises an endogenous MHC α3 domain and/or an endogenous MHC cytoplasmic region.

In some embodiments, the chimeric MHC α chain comprises an amino acid sequence that is at least 70%, 75%, 80%, 85%, 90%, 95%, 99%, or 100% identical to SEQ ID NO: 8, amino acids 25-206 of SEQ ID NO: 8, SEQ ID NO: 59, or amino acids 22-203 of SEQ ID NO: 59.

In some embodiments, the chimeric MHC α chain comprises a α3 domain, a connecting peptide, a transmembrane region, and a cytoplasmic region of an endogenous MHC.

In some embodiments, the animal is a mouse, and the chimeric MHC comprises a α3 domain, a connecting peptide, a transmembrane region, and a cytoplasmic region of mouse H2-D1.

In some embodiments, the chimeric MHC comprises an amino acid sequence that is at least 70%, 75%, 80%, 85%, 90%, 95%, 99%, or 100% identical to amino acids 207-362 of SEQ ID NO: 6.

In some embodiments, the chimeric MHC comprises an amino acid sequence that is at least 70%, 75%, 80%, 85%, 90%, 95%, 99%, or 100% identical to SEQ ID NO: 61 or SEQ ID NO: 63.

In some embodiments, the fusion protein further comprises a signal peptide of human HLA-A2.1 (e.g., at the N-terminus of the fusion protein).

In some embodiments, the signal peptide comprises an amino acid sequence that is at least 70%, 75%, 80%, 85%, 90%, 95%, 99%, or 100% identical to amino acids 1-21 of SEQ ID NO: 59.

In some embodiments, the animal is heterozygous with respect to the sequence encoding the human or humanized MHC α chain or the fusion protein. In some embodiments, the animal is homozygous with respect to the sequence encoding the human or humanized MHC α chain or the fusion protein.

In one aspect, the disclosure is related to a genetically-modified, non-human animal whose genome comprises at least one chromosome comprising a sequence encoding a chimeric MHC α chain comprising a human HLA-A α1 domain, a human HLA-A α2 domain and an endogenous MHC α3 domain.

In some embodiments, the sequence encoding the chimeric MHC α chain is operably linked to an endogenous regulatory element at the endogenous MHC gene locus in the at least one chromosome.

In some embodiments, the genome of the animal further comprises a sequence encoding a human B2M. In some embodiments, the human B2M and the chimeric MHC α chain can associate with each other, forming a functional MHC protein complex in the animal.

In some embodiments, the sequence encoding the human B2M is operably linked to an endogenous regulatory element (e.g., a promoter) at the endogenous B2M gene locus.

In some embodiments, the animal is a mouse, and the sequence encoding the chimeric MHC α chain is operably linked to an endogenous regulatory element (e.g., a promoter) at the mouse H2-D1 gene locus.

In some embodiments, the human HLA-A is human HLA-A2.1.

In one aspect, the disclosure is related to a genetically-modified, non-human animal whose genome comprises at least one chromosome comprising a sequence encoding a human HLA-A.

In some embodiments, the sequence encoding the human HLA-A is operably linked to an endogenous regulatory element at the endogenous MHC gene locus in the at least one chromosome.

In some embodiments, the genome of the animal further comprises a sequence encoding a human B2M. In some embodiments, the human B2M and the human HLA-A can associate with each other, forming a functional MHC protein complex in the animal.

In some embodiments, the sequence encoding the human B2M is operably linked to an endogenous regulatory element (e.g., a promoter) at the endogenous B2M gene locus.

In some embodiments, the animal is a mouse, and the sequence encoding the human HLA-A is operably linked to an endogenous regulatory element (e.g., a promoter) at the mouse H2-D1 gene locus.

In some embodiments, the human HLA-A is human HLA-A2.1.

In some embodiments, the animal does not express endogenous B2M.

In some embodiments, the animal does not express an endogenous MHC molecule (e.g., MHC α chain).

In some embodiments, B2M and the MHC α chain can associate with each other, forming a functional MHC protein complex. In some embodiments, the protein complex can present a non-self antigen to the surface of one or more cells.

In some embodiments, a human T cell (e.g., a cytotoxic T cell) can recognize the presented non-self antigen and initiate immune response. In some embodiments, an endogenous T cells (e.g., a cytotoxic T cell) can recognize the presented non-self antigen and initiate immune response.

In some embodiments, the animal is a mammal, e.g., a monkey, a rodent or a mouse. In some embodiments, the animal is a mouse (e.g., with a C57BL/6 background).

In some embodiments, the genome of the animal comprises a disruption in the animal's endogenous CD132 gene. In some embodiments, the animal is a B-NDG mouse, NOD/scid mouse, a NOD/scid nude mouse, or a B-NDG mouse. In some embodiments, the animal is an immunodeficient mouse.

In some embodiments, the animal further comprises a sequence encoding an additional human or chimeric protein. In some embodiments, the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, SIRPα, CD47, THPO, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α(SIRPα) or TNF Receptor Superfamily Member 4 (OX40).

In one aspect, the disclosure is related to a method for making a genetically-modified, non-human animal, comprising: replacing in at least one cell of the animal, at an endogenous B2M gene locus, a sequence encoding a region of endogenous B2M with a sequence encoding a human B2M or a sequence encoding a fusion protein as described herein.

In some embodiments, the sequence encoding the region of endogenous B2M comprises all or a part of exon 1, exon 2, and exon 3 of endogenous B2M gene.

In one aspect, the disclosure is related to a method for making a genetically-modified, non-human animal, comprising: replacing in at least one cell of the animal, at an endogenous MHC gene locus, a sequence encoding a region of endogenous MHC gene with a sequence encoding a human MHC gene or a sequence encoding a fusion protein.

In some embodiments, the sequence encoding the region of endogenous MHC comprises all or a part of exon 1, exon 2, exon 3, exon 4, exon 5, exon 6, exon 7, and exon 8 of endogenous MHC gene.

In some embodiments, the animal is mouse, and the sequence encoding the region of endogenous MHC comprises all or a part of exon 1, exon 2, exon 3 of mouse H2-D1 gene.

In some embodiments, the sequence encoding the fusion protein comprises the following elements: (a) exon 1, exon 2, and/or exon 3 of human B2M; (b) an optional sequence encoding a linker peptide sequence; and (c) exon 2 and/or exon 3 of human HLA-A2.1. In some embodiments, the sequence encoding the fusion protein further comprises exon 4, exon 5, exon 6, exon 7, and/or exon 8 of endogenous MHC that is downstream of element (c).

In some embodiments, the animal is mouse, and the sequence encoding the fusion protein further comprises the 3′ UTR of mouse H2-D1 gene.

In some embodiments, the fusion protein comprises an amino acid sequence that is at least 70%, 75%, 80%, 85%, 90%, 95%, 99%, or 100% identical to SEQ ID NO: 61, SEQ ID NO: 62, SEQ ID NO: 63, or SEQ ID NO: 64.

In one aspect, the disclosure is related to a method of determining effectiveness of an agent or a combination of agents for the treatment of cancer, comprising: engrafting tumor cells to the animal as described herein, thereby forming one or more tumors in the animal; administering the agent or the combination of agents to the animal; and determining the inhibitory effects on the tumors.

In some embodiments, before engrafting the tumor cells to the animal, human peripheral blood cells (hPBMC) or human hematopoietic stem cells are injected to the animal.

In some embodiments, the tumor cells are from cancer cell lines. In some embodiments, the tumor cells are from a tumor sample obtained from a human patient.

In some embodiments, the inhibitory effects are determined by measuring the tumor volume in the animal.

In some embodiments, the tumor cells are melanoma cells, lung cancer cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

In one aspect, the disclosure is related to a method of producing an animal comprising a human hemato-lymphoid system, the method comprising: engrafting a population of cells comprising human hematopoietic cells or human peripheral blood cells into the animal as described herein.

In some embodiments, the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

In some embodiments, the method described herein further comprises: irradiating the animal prior to the engrafting.

In one aspect, the disclosure is related to a fusion protein comprising β2 microglobulin (B2M) and a human or humanized major histocompatibility complex (MHC) molecule.

In one aspect, the disclosure is related to a nucleic acid encoding the fusion protein as described herein.

In one aspect, the disclosure is related to a protein comprising an amino acid sequence. In some embodiments, the amino acid sequence is one of the following:
(a) an amino acid sequence set forth in SEQ ID NO: 4, 8, 59, 61, 62, 63, or 64;
(b) an amino acid sequence that is at least 90% identical to SEQ ID NO: 4, 8, 59, 61, 62, 63, or 64;
(c) an amino acid sequence that is at least 91%, 92%, 93%, 94%, 95%, 96%, 97%, 98%, or 99% identical to SEQ ID NO: 4, 8, 59, 61, 62, 63, or 64;
(d) an amino acid sequence that is different from the amino acid sequence set forth in SEQ ID NO: 4, 8, 59, 61, 62, 63, or 64 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 4, 8, 59, 61, 62, 63, or 64.

In one aspect, the disclosure is related to a nucleic acid comprising a nucleotide sequence. In some embodiments, the nucleotide sequence is one of the following:
(a) a sequence that encodes the protein as described herein;
(b) SEQ ID NO: 9, 10, 13, 14, 15, 16, 52, 54, or 65;
(c) a sequence that is at least 90% identical to SEQ ID NO: 9, 10, 13, 14, 15, 16, 52, 54, or 65; and
(d) a sequence that is at least 91%, 92%, 93%, 94%, 95%, 96%, 97%, 98%, or 99% identical to SEQ ID NO: 9, 10, 13, 14, 15, 16, 52, 54, or 65.

In one aspect, the disclosure is related to a cell comprising the protein of and/or the nucleic acid as described herein. In one aspect, the disclosure is related to an animal comprising the protein and/or the nucleic acid as described herein.

In one aspect, provided herein is a genetically-modified non-human animal whose genome comprises at least one chromosome comprising a sequence expressing a fusion protein (e.g., a chimeric polypeptide). In some embodiments, the fusion protein comprises all or a part of human B2M protein, and/or all or a part of human MHC molecule (e.g., MHC α chain). In some embodiments, the fusion protein is any one of the fusion protein described in the disclosure.

In some embodiments, the fusion protein comprises at least 50%, at least 60%, at least 70%, at least 80%, at least 90%, or 100% of the protein activity (e.g., antigen-presenting) of a wildtype human MHC molecule (e.g., HLA-A) In some embodiments, the sequence encoding the fusion protein is operably linked to an endogenous regulatory element (e.g., a promoter) at the endogenous B2M gene locus in the at least one chromosome. In some embodiments, the sequence encoding the fusion protein is operably linked to an endogenous regulatory element (e.g., a promoter) at the endogenous MHC gene locus in the at least one chromosome. In some embodiments, the animal is a mouse, and the sequence encoding the fusion protein is operably linked to an endogenous regulatory element at the mouse H2-D1 gene locus in the at least one chromosome.

In another aspect, the disclosure relates to a non-human mammalian cell, comprising a disruption, a deletion, or a genetic modification as described herein.

In some embodiments, the cell includes Cas9 mRNA or an in vitro transcript thereof.

In some embodiments, the non-human mammalian cell is a mouse cell. In some embodiments, the cell is a fertilized egg cell. In some embodiments, the cell is a germ cell. In some embodiments, the cell is a blastocyst.

In another aspect, the disclosure relates to a tumor bearing non-human mammal model, characterized in that the non-human mammal model is obtained through the methods as described herein.

The disclosure also relates to a cell or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Immunodeficient animals are an indispensable research tool for studying the mechanism of diseases, and methods of treating such diseases. They can easily accept xenogeneic cells or tissues due to their immunodeficiency, and have been widely used in the research. The commonly used immunodeficient animals include e.g., NOD-PrkdC^{scid}IL-2rγ^{null} mice, NOD-Rag 1^{-/-}-IL2rg^{-/-} (NRG), Rag 2^{-/-} IL2rg^{-/-} (RG), NOD/SCID (NOD-Prkdc^{scid}), and NOD/SCID nude mice. Among them, NOD-Prkdc^{scid} II-2rγ^{null} mice may be the best recipient mice for transplantation. Some of these mice are described in detail e.g., in Ito et al. “Current advances in humanized mouse models.” Cellular & molecular immunology 9.3 (2012): 208; and US20190320631A1, each of which is incorporated herein by reference in its entirety.

It has been well established that T cells recognize antigen in association with self MHC proteins but not in association with foreign MHC proteins: that is, T cells show MHC restriction. This restriction results from a process of positive selection during T cell development in the thymus. In this process, those immature T cells that will be capable of recognizing foreign peptides presented by self MHC proteins are selected to survive, while the remainder, which would be of no use to the animal, undergo apoptosis. Thus, MHC restriction is an acquired property of the immune system that emerges as T cells develop in the thymus. However, in these immunodeficient animals (e.g., NOD-Prkdc^{scid} IL-2rγ^{null}) the MHC of immunodeficient animals are still the endogenous MHC. After transplantation of human cells or tissues, human-derived immune cells cannot go through the restriction process mediated by human HLA in the animal's thymus, and cannot reflect the restriction of human MHC after immunotherapy or infection of pathogens. Thus, in some cases, the transplanted human T and B cells are not fully functionally mature in these immunodeficient animals.

This disclosure relates to genetically modified animals which express a human or chimeric (e.g., humanized) major histocompatibility complex (MHC) protein complex, and methods of use thereof. In some embodiments, the animal is an immunodeficient animal (e.g., with NOD-Prkdc^{scid} IL-2rγ^{null}, NOD-Rag 1^{-/-}-IL2rg^{-/-} (NRG), Rag 2^{-/-} IL2rg^{-/-} (RG), or NOD/SCID (NOD-Prkdc^{scid}) background). The human or humanized MHC provides a better environment for the transplanted human immune cells (e.g., T cells and B cells) to mature, and particularly these animals provide a more actuate model for determining the efficacy of various therapies for human.

### Major Histocompatibility Complex

The major histocompatibility complex (MHC) plays a key role in the defense mechanism of a body induced by T cell immune responses by presenting a cancer- or virus-derived antigen peptide. MHC is classified as class I or class II. Class I is expressed in all somatic cells except for germ cells and erythrocytes. MHC class I protein complex is composed of an α chain (alpha chain or heavy chain) and a smaller chain known as β2 microglobulin (B2M). The α chain is composed of α1 and α2 domains associated with the formation of an antigen peptide-holding groove and an α3 domain associated with the binding to a co-receptor CD8 molecule expressed on the cytotoxic T cell (CTL) surface. Like MHC class I molecules, class II molecules are also heterodimers, and have two peptides, α and β chains.

Both humans and mice have MHC class I and class II genes. In humans, the classical class I genes are termed HLA-A, HLA-B and HLA-C, whereas in mice they are H-2K, H-2D and H-2L. In Class I molecules, the α-chain (also known as heavy chain) is polymorphic, and the smaller chain B2M (also known as light chain) is generally not polymorphic. The α-chain contains three domains (α1, α2 and α3). Usually, exon 1 of the α-chain gene encodes the leader sequence, exons 2 and 3 encode the α1 and α2 domains, exon 4 encodes the α3 domain, exon 5 encodes the transmembrane domain, and exons 6 and 7 encode the cytoplasmic tail. The α-chain forms a peptide-binding cleft involving the α1 and α2 domains (which resemble Ig-like domains) followed by the α3 domain, which is similar to β2-microglobulin.

Class I MHC are expressed on all nucleated cells, including tumor cells. They are expressed specifically on T and B lymphocytes, macrophages, dendritic cells and neutrophils, among other cells, and function to display peptide fragments (typically 8-10 amino acids in length) on the surface to CD8+ cytotoxic T lymphocytes (CTLs). CTLs are specialized to kill any cell that bears an MHC I-bound peptide recognized by its own membrane-bound TCR. When a cell displays peptides derived from cellular proteins not normally present (e.g., of viral, tumor, or other non-self origin), such peptides are recognized by CTLs, which become activated and kill the cell displaying the peptide.

This disclosure relates to genetically modified animals which express a human or chimeric (e.g., humanized) MHC protein complex, MHC Class I polypeptide, or B2M. As used herein, the term “MHC I complex” or “MHC Class I complex” refers to the complex formed by the MHC I α chain polypeptide and the B2M polypeptide. In some embodiments, the MHC I α chain polypeptide and the B2M polypeptide are fused together. The term “MHC I polypeptide” or “MHC Class I polypeptide,” as used herein, refers to the MHC I α chain polypeptide alone.

### β2 Microglobulin (B2M)

As discussed above, a wildtype MHC class I molecules are heterodimers that consist of two polypeptide chains, α and β2-microglobulin (B2M) (FIG. 1). The two chains are linked noncovalently via interaction of B2M and the αβ domain. Only the α chain is polymorphic and encoded by a HLA gene. The B2M subunit is not polymorphic and is encoded by the B2M gene. The α3 domain is plasma membrane-spanning and interacts with the CD8 co-receptor of T-cells. The α3-CD8 interaction holds the MHC I molecule in place while the T cell receptor (TCR) on the surface of the cytotoxic T cell binds its α1-α2 heterodimer ligand, and checks the coupled peptide for antigenicity. The α1 and α2 domains fold to make up a groove for peptides to bind. MHC class I molecules bind peptides that are predominantly 8-10 amino acid in length.

B2M (also known as β2M, β₂ microglobulin or beta-2 microglobulin) is a small protein (about 11,800 Dalton), presenting in nearly all nucleated cells and most biological fluids, including serum, urine, and synovial fluid. The human β2M shows 70% amino acid sequence similarity to the murine protein and both of them are located on the syntenic chromosomes. The secondary structure of B2M consists of seven β-strands which are organized into two β-sheets linked by a single disulfide bridge, presenting a classical β-sandwich typical of the immunoglobulin (Ig) domain. B2M has no transmembrane region and contains a distinctive molecular structure called a constant-1 Ig superfamily domain, sharing with other adaptive immune molecules including major histocompatibility complex (MHC) class I and class II. Two evolutionary conserved tryptophan (Trp) residues are important for correct structural fold and function of B2M. Trp60 is exposed to the solvent at the apex of a protein loop and is critical for promoting the association of B2M in MHC I.

Normally, B2M is noncovalently linked with the other polypeptide chain (α chain) to form MHC I or like structures, including MHC I, neonatal Fc receptor (FcRn), a cluster of differentiation 1 (CD1), human hemochromatosis protein (HFE), Qa, and so on. B2M makes extensive contacts with all three domains of the α chain. Thus, the conformation of α chain is highly dependent on the presence of B2M. Although α1 and α2 domains differ among molecules, α3 domain and B2M are relatively conserved, where the intermolecular interaction occurs. A number of residues at the points of contact with B2M are shared among MHC I or like molecules. Furthermore, interactions with α1 and α2 domains are important for the paired association of α3 domain and B2M in the presence of native antigens. B2M can dissociate from such molecules and shed into the serum, where it is transported to the kidneys to be degraded and excreted. An 88-kD protein (calnexin) associates rapidly and quantitatively with newly synthesized murine MHC I molecules within the endoplasmic reticulum. Both B2M and peptide are required for efficient calnexin dissociation and subsequent MHC I transport.

B2M can stabilize the tertiary structure of the MHC I or like molecules. It is also extensively involved in the functional regulation of survival, proliferation, apoptosis, and even metastasis in cancer cells.

A detailed description of B2M and its function can be found, e.g., in Li et al., “The implication and significance of beta 2 microglobulin: A conservative multifunctional regulator.” Chinese medical journal 129.4 (2016): 448; Wang et al., “Targeted Disruption of the 02-Microglobulin Gene Minimizes the Immunogenicity of Human Embryonic Stem Cells,” Stem cells translational medicine 4.10 (2015): 1234-1245; each of which is incorporated herein by reference in its entirety.

In human genomes, B2M gene (Gene ID: 567) locus has four exons, exon 1, exon 2, exon 3, and exon 4 (FIG. 2). The B2M protein also has a signal peptide. The nucleotide sequence for human B2M mRNA is NM_004048.4 (SEQ ID NO: 3), and the amino acid sequence for human B2M is NP_004039.1 (SEQ ID NO: 4). The location for each exon and each region in human B2M nucleotide sequence and amino acid sequence is listed below.

TABLE 1

| | NM_004048.3 | NP_004039.1 |
|---|---|---|
| Human B2M | 1675 bp | 119 aa |
| (approximate location) | (SEQ ID NO: 3) | (SEQ ID NO: 4) |
| Exon 1 | 1-97 | 1-22 |
| Exon 2 | 98-376 | 23-115 |
| Exon 3 | 377-404 | 116-119 |
| Exon 4 | 405-1675 | Non-coding |
| Signal peptide | 31-90 | 1-20 |
| Donor region in Example | 91-387 | 21-119 |

The Human B2M gene (Gene ID: 567) is located in Chromosome 15 of the human genome, which is located from 44,711,487 to 44,718,877, of NC_000015.10 (GRCh38.p13 (GCF_000001405.39)). The 5′-UTR is from 44,711,517 to 44,711,546, exon 1 is from 44,711,614 to 44,711,613, the first intron is from 44,711,614 to 44,715,422, exon 2 is from 44,715,423 to 44,715,701, the second intron is from 44,715,702 to 44,716,328, exon 3 is from 44,716,329 to 44,716,356, the third intron is from 44,716,357 to 44,717,606, exon 4 is from 44,717,607 to 44,718,145, the 3′-UTR is from 44,716,343 to 44,716,356 and 44,717,607 to 44,718,145, based on transcript NM_004048.3. All relevant information for human B2M locus can be found in the NCBI website with Gene ID: 567, which is incorporated by reference herein in its entirety.

In mice, B2M gene locus has four exons, exon 1, exon 2, exon 3, and exon 4 (FIG. 2). The mouse B2M protein also has a signal peptide. The nucleotide sequence for mouse B2M mRNA is NM_009735.3 (SEQ ID NO: 1), the amino acid sequence for mouse B2M is NP_033865.2 (SEQ ID NO: 2). The location for each exon and each region in the mouse B2M nucleotide sequence and amino acid sequence is listed below:

TABLE 2

| | NM_009735.3 | NP_033865.2 |
|---|---|---|
| Mouse B2M | 858 bp | 119 aa |
| (approximate location) | (SEQ ID NO: 1) | (SEQ ID NO: 2) |
| Exon 1 | 1-118 | 1-22 |
| Exon 2 | 119-397 | 23-115 |
| Exon 3 | 398-426 | 116-119 |
| Exon 4 | 427-858 | Non-coding |
| Signal peptide | 52-111 | 1-20 |
| Replaced region in Examples | 52-426 | 1-119 |

The mouse B2m gene (Gene ID: 12010) is located in Chromosome 2 of the mouse genome, which is located from 122,147,686 to 122,153,083, of NC_000068.7 (GRCm38.p6 (GCF_000001635.26)). The 5′-UTR is from 122,147,686 to 122,147,736, exon 1 is from 122,147,686 to 122,147,804, the first intron is from 122,147,805 to 122,150,872, exon 2 is from 122,150,873 to 122,151,151, the second intron is from 122,151,152 to 122,151,646, exon 3 is from 122,151,647 to 122,151,675, the third intron is from 122,151,676 to 122,152,650, exon 4 is from 122,152,651 to 122,153,083, the 3′-UTR is from 122,151,661 to 122,153,083, based on transcript NM_009735.3. All relevant information for mouse B2m locus can be found in the NCBI website with Gene ID: 12010, which is incorporated by reference herein in its entirety.

FIG. 23 shows the alignment between mouse B2M amino acid sequence (NP_033865.2; SEQ ID NO: 2) and human B2M amino acid sequence (NP_004039.1; SEQ ID NO: 4). Thus, the corresponding amino acid residue or region between human and mouse B2M can be found in FIG. 23.

B2M genes, proteins, and locus of the other species are also known in the art. For example, the gene ID for B2M in Rattus norvegicus (rat) is 24223, the gene ID for B2M in Macaca mulatta (Rhesus monkey) is 712428, the gene ID for B2M in Equus caballus (horse) is 100034203, and the gene ID for B2M in Sus scrofa (pig) is 397033. The relevant information for these genes (e.g., intron sequences, exon sequences, amino acid residues of these proteins) can be found, e.g., in NCBI database, which is incorporated by reference herein in its entirety. FIG. 24 shows the alignment between rodent B2M amino acid sequence (NP_036644.1; SEQ ID NO: 66) and human B2M amino acid sequence (NP_004039.1; SEQ ID NO: 4). Thus, the corresponding amino acid residue or region between human and rodent B2M can be found in FIG. 24.

The present disclosure provides human or chimeric (e.g., humanized) B2M nucleotide sequence and/or amino acid sequences. In some embodiments, the entire sequence of mouse exon 1, exon 2, exon 3, exon 4, and/or signal peptide, are replaced by the corresponding human sequence. In some embodiments, a “region” or “portion” of mouse exon 1, exon 2, exon 3, exon 4, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, or 110 amino acid residues.

In some embodiments, the “region” or “portion” can be at least 50%, 55%, 60%, 65%, 70%, 75%, 80%, 85%, 90%, 95%, or 99% identical to exon 1, exon 2, exon 3, exon 4, or signal peptide. In some embodiments, a region, a portion, or the entire sequence of mouse exon 1, exon 2, exon 3, and/or exon 4 (e.g., a part of exon 1, exon 2, and a part of exon 3) are replaced by a region, a portion, or the entire sequence of the human exon 1, exon 2, exon 3, and/or exon 4 (e.g., a part of exon 1, exon 2, and a part of exon 3) sequence.

In some embodiments, the present disclosure is related to a genetically-modified, non-human animal whose genome comprises a chimeric (e.g., humanized) B2M nucleotide sequence. In some embodiments, the chimeric (e.g., humanized) B2M nucleotide sequence encodes a B2M protein comprising a signal peptide. In some embodiments, the signal peptide described herein is at least 80%, 85%, 90%, 95%, or 100% identical to amino acids 1-22 of SEQ ID NO: 2. In some embodiments, the signal peptide described herein is at least 80%, 85%, 90%, 95%, or 100% identical to amino acids 1-22 of SEQ ID NO: 4. In some embodiments, the humanized protein has a sequence that is at least 80%, 85%, 90%, 95%, or 100% identical to amino acids 1-119 or 23-119 of SEQ ID NO: 2. In some embodiments, the humanized protein has a sequence that is at least 80%, 85%, 90%, 95%, or 100% identical to amino acids 1-119, 23-119, or 21-119 of SEQ ID NO: 4.

In some embodiments, the present disclosure also provides a chimeric (e.g., humanized) B2M nucleotide sequence and/or amino acid sequences, wherein in some embodiments, at least 1%,2%, 3%, 4%, 5%, 6%, 7%, 8%, 9%, 10%, 10%15%, 20%,25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse B2M mRNA sequence (e.g., SEQ ID NO: 1), mouse B2M amino acid sequence (e.g., SEQ ID NO: 2), or a portion thereof (a portion of exon 1, exon 2, and a portion of exon 3); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human B2M mRNA sequence (e.g., SEQ ID NO: 3), human B2M amino acid sequence (e.g., SEQ ID NO: 4), or a portion thereof (e.g., a portion of exon 1, exon 2, and a portion of exon 3).

In some embodiments, the sequence encoding a region of mouse B2M (e.g., amino acids 1-119 of SEQ ID NO: 2) is replaced. In some embodiments, the sequence is replaced by a sequence encoding a corresponding region of human B2M (e.g., amino acids 1-119 of human B2M (SEQ ID NO: 4)).

In some embodiments, the nucleic acids as described herein are operably linked to a promotor or regulatory element, e.g., an endogenous mouse B2M promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse B2M nucleotide sequence (e.g., exon 1, exon 2, exon 3, exon 4, or NM_009735.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse B2M nucleotide sequence (e.g., exon 1, exon 2, exon 3, exon 4, or NM_009735.3 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human B2M nucleotide sequence (e.g., exon 1, exon 2, exon 3, exon 4, or NM_004048.4 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human B2M nucleotide sequence (e.g., exon 1, exon 2, exon 3, exon 4, or NM_004048.4 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse B2M amino acid sequence (e.g., amino acids encoded by exon 1, exon 2, exon 3, and/or exon 4 of NM_009735.3 (SEQ ID NO: 1); or NP_033865.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse B2M amino acid sequence (e.g., amino acids encoded by exon 1, exon 2, exon 3, and/or exon 4 of NM_009735.3 (SEQ ID NO: 1); or NP_033865.2 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human B2M amino acid sequence (e.g., amino acids encoded by exon 1, exon 2, exon 3, and/or exon 4 of NM_004048.4 (SEQ ID NO: 3); or NP_004039.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human B2M amino acid sequence (e.g., amino acids encoded by exon 1, exon 2, exon 3, and/or exon 4 of NM_004048.4 (SEQ ID NO: 3); or NP_004039.1 (SEQ ID NO: 4)).

### Human Leukocyte Antigen (HLA)

Human leukocyte antigens (HLAs) corresponding to MHC class I molecules include, e.g., HLA-A, HLA-B, and HLA-C. Human HLAs corresponding to MHC class II molecules include, e.g., HLA-DP, HLA-DM, HLA-DO, HLA-DQ, and HLA-DR.

Human HLA-A can have many serotype groups, e.g., HLA-A1 and HLA-A*02. For HLA-A1 (A1), the serotype is determined by the antibody recognition of α1 subset of HLA-A α-chains. For A1, the α chain is encoded by the HLA-A*01 allele group and the β-chain is encoded by B2M locus. This group currently is dominated by A*0101 (A*01:01:01:01). For HLA-A*02 (HLA-A2), the serotype is determined by the antibody recognition of the α2 domain of the HLA-A α-chain. For A*02, the α chain is encoded by the HLA-A*02 gene and the β chain is encoded by the B2M locus. A subtype of HLA-A2 is HLA-A2.1. Details of HLA nomenclature can be found, e.g., in Marsh, S. G. et al., “Nomenclature for factors of the HLA system, 2010.” Tissue antigens 75.4 (2010): 291, which is incorporated herein by reference in its entirety.

In human genomes, a typical HLA-A gene locus has eight exons, exon 1, exon 2, exon 3, exon 4, exon 5, exon 6, exon 7, and exon 8 (FIG. 3). The HLA-A protein also has a signal peptide, an extracellular region, a transmembrane region, and a cytoplasmic region. Further, the extracellular region includes an α1 domain, an α2 domain, an α3 domain, and a connecting peptide. The nucleotide sequence for human HLA-A*0101 mRNA is NM_001242758.1 (SEQ ID NO: 7), and the amino acid sequence for human HLA-A*0101 is NP_001229687.1 (SEQ ID NO: 8). In addition, the nucleotide sequence for human HLA-A2.1 is nucleic acids 95493-99436 of AF055066.1 (SEQ ID NO: 54), and the amino acid sequence for human HLA-A2.1 is AAC24825.1 (SEQ ID NO: 59). The location for each exon and each region in human HLA-A nucleotide sequence and amino acid sequence is listed below.

TABLE 3

| | NM_001242758.1 | NP_001229687.1 | AAC24825.1 |
|---|---|---|---|
| Human HLA-A | 1611bp | 365 aa | 362aa |
| (approximate | (SEQ ID | (SEQ ID | (SEQ ID |
| location) | NO: 7) | NO: 8) | NO: 59) |
| Exon 1 | 1-157 | 1-24 | 1-21 |
| Exon 2 | 158-427 | 25-114 | 22-111 |
| Exon 3 | 428-703 | 115-206 | 112-203 |
| Exon 4 | 704-979 | 207-298 | 204-295 |
| Exon 5 | 980-1096 | 299-337 | 296-334 |
| Exon 6 | 1097-1129 | 338-348 | 335-345 |
| Exon 7 | 1130-1177 | 349-364 | 346-362 |
| Exon 8 | 1178-1611 | 365 | Non-coding |
| Signal peptide | 85-156 | 1-24 | 1-21 |
| Extracellular | 157-1008 | 25-308 | 22-305 |
| Transmembrane | 1009-1080 | 309-332 | 306-329 |
| Cytoplasmic | 1081-1611 | 333-365 | 330-362 |
| Alpha-1 | 157-426 | 25-114 | 22-111 |
| Alpha-2 | 427-702 | 115-206 | 112-203 |
| Alpha-3 | 703-978 | 207-298 | 204-295 |
| Connecting | 979-1008 | 299-308 | 296-305 |
| peptide | | | |
| Donor region | NA | NA | 1-203 |
| in Example 1 | | | (C57BL/B6 |
| | | | background) |
| Donor region | NA | NA | 1-362 |
| in Example 2 | | | (B-NDG |
| | | | background) |

Human MHC class I region (GenBank ID: AF055066.1) is located in Chromosome 6 of the human genome. The Human HLA-A2.1 gene is located from 95493 to 99436 of AF055066.1. Exon 1 is from 99566 to 99629, the first intron is from 99436 to 99565, exon 2 is from 99166 to 99435, the second intron is from 98925 to 99165, exon 3 is from 98649 to 98924, the third intron is from 98049 to 98648, exon 4 is from 97773 to 98048, the fourth intron is from 97674 to 97772, exon 5 is from 97557 to 97673, the fifth intron is from 97119 to 97556, exon 6 is from 97086 to 97118, the sixth intron is from 97085 to 96944, exon 7 is from 96896 to 96943, the seventh intron is from 96727 to 96895, exon 8 is from 96726 to 96322, the 3′-UTR is from 96721 to 96322, based on AF055066.1. All relevant information for human HLA-A2.1 gene locus can be found in the NCBI website with GenBank ID: AF055066.1, which is incorporated by reference herein in its entirety.

In mice, H-2K, H-2D and H-2L are MHC class I genes. Particularly, the H2-D1 gene locus has eight exons, exon 1, exon 2, exon 3, exon 4, exon 5, exon 6, exon 7 and exon 8 (FIG. 3). The mouse H2-D1 protein (encoding MHC class I α chain) also has a signal peptide, an extracellular region, a transmembrane region, and a cytoplasmic region. Specifically, the extracellular region includes an α1 domain, an α2 domain, an α3 domain, and a connecting peptide. The nucleotide sequence for mouse H2-D1 mRNA is NM_010380.3 (SEQ ID NO: 5), the amino acid sequence for mouse H2-D1 is NP_034510.3 (SEQ ID NO: 6). The location for each exon and each region in the mouse H2-D1 nucleotide sequence and amino acid sequence is listed below:

TABLE 4

| | NM_010380.3 | NP_034510.3 |
|---|---|---|
| Mouse H2-D1 | 1736 bp | 362aa |
| (approximate location) | (SEQ ID NO: 5) | (SEQ ID NO: 6) |
| Exon 1 | 1-93 | 1-24 |
| Exon 2 | 94-363 | 25-114 |
| Exon 3 | 364-639 | 115-206 |
| Exon 4 | 640-915 | 207-298 |
| Exon 5 | 916-1032 | 299-337 |
| Exon 6 | 1033-1065 | 338-348 |
| Exon 7 | 1066-1104 | 349-361 |
| Exon 8 | 1105-1736 | 362 |
| Signal peptide | 21-92 | 1-24 |
| Extracellular | 93-947 | 25-309 |
| Transmembrane | 948-1013 | 310-331 |
| Cytoplasmic | 1014-1736 | 332-362 |
| Alpha-1 | 93-362 | 25-114 |
| Alpha-2 | 363-638 | 115-206 |
| Alpha-3 | 639-914 | 207-298 |
| Connecting peptide | 915-947 | 299-309 |
| Donor region in Examples | 640-1736 | 207-362 |

The mouse H2-D1 gene (Gene ID: 14964; MGI: 95896) is located in Chromosome 17 of the mouse genome, which is located from 35482070 to 35486473 of NC_000083.7 (GRCm39 (GCF_000001635.27)). The 5′-UTR is from 35,262,730 to 35,263,113, exon 1 is from 35,262,730 to 35,263,186, the first intron is from 35,263,187 to 35,263,378, exon 2 is from 35,263,379 to 35,263,648, the second intron is from 35,263,649 to 35,263,838, exon 3 is from 35,263,839 to 35,264,114, the third intron is from 35,264,115 to 35,265,783, exon 4 is from 35,265,784 to 35,266,059, the forth intron is from 35,266,060 to 35,266,186, exon 5 is from 35,266,187 to 35,266,303, the fifth intron is from 35,266,304 to 35,266,481, exon 6 is from 35,266,482 to 35,266,514, the sixth intron is from 35,266,515 to 35,266,687, exon 7 is from 35,266,688 to 35,266,726, the seventh intron is from 35,266,727 to 35,266,865, exon 8 is from 35,266,866 to 35,267,499, the 3′-UTR is from 35,266,871 to 35,267,499, based on transcript NM_010380.3. All relevant information for mouse H2-D1 locus can be found in the NCBI website with Gene ID: 14964, which is incorporated by reference herein in its entirety.

FIG. 25 shows the alignment between mouse H2-D1 amino acid sequence (NP_034510.3; SEQ ID NO: 6) and human HLA-A2.1 amino acid sequence (AAC24825.1; SEQ ID NO: 59). Thus, the corresponding amino acid residue or region between human HLA-A2.1 and mouse H2-D1 can be found in FIG. 25.

FIG. 26 shows the alignment between mouse H2-D1 amino acid sequence (NP_034510.3; SEQ ID NO: 6) and human HLA-A*0101 amino acid sequence (NP_001229687.1; SEQ ID NO: 8). Thus, the corresponding amino acid residue or region between human HLA-A*0101 and mouse H2-D1 can be found in FIG. 26.

MHC molecule genes, proteins, and locus of the other species are also known in the art. For example, the gene ID and the relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) MHC molecule (e.g., MHC class I alpha chain) nucleotide sequence and/or amino acid sequences. This disclosure also relates to genetically modified animals which express a human or chimeric (e.g., humanized) HLA-A protein complex and/or HLA-A polypeptide. As used herein, the term “HLA-A complex” or “HLA-A protein complex” refers to the complex formed by the HLA-A α chain polypeptide and the B2M polypeptide. In some embodiments, the HLA-A α chain polypeptide and the B2M polypeptide are fused together. The term “HLA-A” or “HLA-A polypeptide” as used herein refers to the HLA-A α chain polypeptide.

In some embodiments, the entire sequence of mouse H2-D1 exon 1, exon 2, exon 3, exon 4, exon 5, exon 6, exon 7, exon 8, signal peptide, extracellular region (e.g., α1 domain, α2 domain, α3 domain, and/or connecting peptide), transmembrane region, and/or cytoplasmic region are replaced by the corresponding human sequence. In some embodiments, a “region” or “portion” of mouse H2-D1 exon 1, exon 2, exon 3, exon 4, exon 5, exon 6, exon 7, exon 8, signal peptide, extracellular region (e.g., α1 domain, α2 domain, α3 domain, and/or connecting peptide), transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, or 360 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region (e.g., α1 domain, α2 domain, α3 domain, and/or connecting peptide), transmembrane region, and/or cytoplasmic region. In some embodiments, a region, a portion, or the entire sequence of mouse H2-D1 exon 1, exon 2, exon 3, exon 4, exon 5, exon 6, exon 7, and/or exon 8 (e.g., exon 1, exon 2, and exon 3) are replaced by a region, a portion, or the entire sequence of the human HLA-A exon 1, exon 2, exon 3, exon 4, exon 5, exon 6, exon 7, and/or exon 8 (e.g., exon 1, exon 2, exon 3) sequence.

In some embodiments, the present disclosure is related to a genetically-modified, non-human animal whose genome comprises a chimeric (e.g., humanized) MHC molecule (e.g., human HLA/mouse H2-D1) nucleotide sequence. In some embodiments, the chimeric (e.g., humanized) MHC molecule nucleotide sequence encodes a MHC molecule protein comprising an extracellular region, a transmembrane region, a cytoplasmic region, and a signal peptide. In some embodiments, the extracellular region comprises the entire or part of human HLA-A (e.g., HLA-A*0101, or HLA-A2.1) extracellular region. For example, the extracellular region described herein comprises an amino acid sequence that is at least 80%, 85%, 90%, 95%, or 100% identical to human HLA-A extracellular region (e.g., amino acids 25-308 of SEQ ID NO:8, or amino acids 22-305 of SEQ ID NO: 59). In some embodiments, the transmembrane region comprises the entire or part of human HLA-A (e.g., HLA-A*0101, or HLA-A2.1) transmembrane region. For example, the transmembrane region is at least 80%, 85%, 90%, 95%, or 100% identical to human HLA transmembrane region (e.g., amino acids 309-332 of SEQ ID NO:8, or amino acids 306-329 of SEQ ID NO: 59). In some embodiments, the cytoplasmic region comprises the entire or part of human HLA-A (e.g., HLA-A*0101, or HLA-A2.1) cytoplasmic region. For example, the cytoplasmic region is at least 80%, 85%, 90%, 95%, or 100% identical to human HLA extracellular region (e.g., amino acids 333-365 of SEQ ID NO:8, or amino acids 330-362 of SEQ ID NO: 59).

In some embodiments, the chimeric (e.g., humanized) MHC molecule nucleotide sequence encodes a MHC molecule protein comprising a signal peptide. In some embodiments, the signal peptide described herein is at least 80%, 85%, 90%, 95%, or 100% identical to amino acids 1-24 of SEQ ID NO: 6. In some embodiments, the signal peptide described herein is at least 80%, 85%, 90%, 95%, or 100% identical to amino acids 1-24 of SEQ ID NO: 8, or amino acids 1-21 of SEQ ID NO: 59.

In some embodiments, the present disclosure also provides a chimeric (e.g., humanized) MHC molecule nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse H2-D1 mRNA sequence (e.g., SEQ ID NO: 5), mouse H2-D1 amino acid sequence (e.g., SEQ ID NO: 6), or a portion thereof (e.g., exon 4, exon 5, exon 6, exon 7, and a portion of exon 8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human HLA-A molecule mRNA sequence (e.g., SEQ ID NO: 7), human HLA-A amino acid sequence (e.g., SEQ ID NO: 8 or SEQ ID NO: 59), or a portion thereof (e.g., a portion of exon 1, exon 2, and exon 3).

In some embodiments, the sequence encoding a region of mouse H2-D1 (e.g., amino acids 1-206 or 25-206 of SEQ ID NO: 6) is replaced. In some embodiments, the sequence is replaced by a sequence encoding a corresponding region of human HLA-A (e.g., amino acids 1-206 or 25-206 of human HLA-A*0101 (SEQ ID NO: 8); or amino acids 1-203 or 22-203 of human HLA-A2.1 (SEQ ID NO: 59)).

In some embodiments, the nucleic acids as described herein are operably linked to a promotor or regulatory element, e.g., an endogenous mouse H2-D1 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse H2-D1 nucleotide sequence (e.g., exon 1, exon 2, exon 3, exon 4, exon 5, exon 6, exon 7, exon 8, or NM_010380.3 (SEQ ID NO: 5)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse H2-D1 nucleotide sequence (e.g., exon 1, exon 2, exon 3, exon 4, exon 5, exon 6, exon 7, exon 8, or NM_010380.3 (SEQ ID NO: 5)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human HLA-A nucleotide sequence (e.g., exon 1, exon 2, exon 3, exon 4, exon 5, exon 6, exon 7, exon 8, or NM_001242758.1 (SEQ ID NO: 7)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human HLA-A nucleotide sequence (e.g., exon 1, exon 2, exon 3, exon 4, exon 5, exon 6, exon 7, exon 8, or NM_001242758.1 (SEQ ID NO: 7)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse H2-D1 amino acid sequence (e.g., amino acids encoded by exon 1, exon 2, exon 3, exon 4, exon 5, exon 6, exon 7, and/or exon 8 of NM_010380.3 (SEQ ID NO: 5); or NP_034510.3 (SEQ ID NO: 6)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse H2-D1 amino acid sequence (e.g., amino acids encoded by exon 1, exon 2, exon 3, exon 4, exon 5, exon 6, exon 7, and/or exon 8 of NM_010380.3 (SEQ ID NO: 5); or NP_034510.3 (SEQ ID NO: 6)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human HLA-A amino acid sequence (e.g., amino acids encoded by exon 1, exon 2, exon 3, exon 4, exon 5, exon 6, exon 7, and/or exon 8 of NM_001242758.1 (SEQ ID NO: 8); NP_001229687.1 (SEQ ID NO: 8); or AAC24825.1 (SEQ ID NO: 59)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human HLA-A amino acid sequence (e.g., amino acids encoded by exon 1, exon 2, exon 3, exon 4, exon 5, exon 6, exon 7, and/or exon 8 of NM_001242758.1 (SEQ ID NO: 8); NP_001229687.1 (SEQ ID NO: 8); or AAC24825.1 (SEQ ID NO: 59)).

The present disclosure further relates to a B2M or MHC molecule genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 9, 10, 13, 14, 15, 16, 52, 54, or 65.

The disclosure also provides an amino acid sequence that has a homology of at least 90% with, or at least 90% identical to the sequence shown in SEQ ID NO: 4, 8, 59, 61, 62, 63, or 64, and has protein activity. In some embodiments, the homology with the sequence shown in SEQ ID NO: 4, 8, 59, 61, 62, 63, or 64 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 4, 8, 59, 61, 62, 63, or 64 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 9, 10, 13, 14, 15, 16, 52, 54, or 65, and encodes a polypeptide that has protein activity. In some embodiments, the homology with the sequence shown in SEQ ID NO: 9, 10, 13, 14, 15, 16, 52, 54, or 65 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 9, 10, 13, 14, 15, 16, 52, 54, or 65 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) MHC from an endogenous non-human B2M or MHC gene locus.

### Genetically Modified Animals

As used herein, the term “genetically-modified non-human animal” refers to a non-human animal having a modified sequence (e.g., replacement of endogenous B2M gene with a sequence encoding the fusion protein described herein or a sequence encoding the humanized B2M described herein, and/or replacement of endogenous MHC gene (e.g., MHC class I α chain) with a sequence encoding the fusion protein described herein or a sequence encoding the humanized MHC gene described herein) in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the modified sequence in its genome. The cell having the modified sequence can be various kinds of cells, e.g., an endogenous cell, a somatic cell, an immune cell, a T cell, a B cell, a germ cell, a blastocyst, or an endogenous tumor cell. In some embodiments, genetically-modified non-human animals are provided that comprise a human or humanized B2M and/or human or humanized MHC molecule gene (e.g., MHC class I α chain) at the endogenous B2M or MHC gene locus. The animals are generally able to pass the modification to progeny, i.e., through germline transmission.

As used herein, the term “humanized” and the like refers to a molecule (e.g., a nucleic acid, protein, etc.) that was non-human in origin and for which a portion has been replaced with a corresponding portion of a corresponding human molecule in such a manner that the modified (e.g., humanized) molecule retains its biological function and/or maintains the structure that performs the retained biological function. A humanized molecule may be considered derived from a human molecule where the humanized molecule is encoded by a nucleotide comprising a nucleic acid sequence that encodes the human molecule (or a portion thereof).

In some embodiments, the genetically-modified non-human animal does not express an endogenous B2M (e.g., mouse B2M). In some embodiments, the genetically-modified non-human animal does not express a functional endogenous B2M (e.g., mouse B2M). In some embodiments, the genetically-modified non-human animal does not express an endogenous MHC molecule (e.g., mouse H2-D1). In some embodiments, the genetically-modified non-human animal does not express a functional endogenous MHC molecule (e.g., mouse H2-D1) or a functional endogenous MHC protein complex.

In some embodiments, the genetically-modified non-human animal described herein is immunodeficient. In some embodiments, the animal has a NOD-Prkdc^{scid} IL-2rγ^{nul}, NOD-Rag 1^{-/-}-IL2rg^{-/-} (NRG), Rag 2^{-/-}-IL2rg^{-/-} (RG), or NOD/SCID (NOD-Prkdc^{scid}) background.

In some embodiments, the genetically-modified non-human animal described herein (e.g., mouse) have a disrupted endogenous B2M gene. In some embodiments, the genetically-modified non-human animal described herein (e.g., mouse) expresses a dysfunctional endogenous B2M protein (e.g., mouse B2M). In some embodiments, the genetically-modified non-human animal described herein (e.g., mouse) have a disrupted endogenous MHC gene. In some embodiments, the genetically-modified non-human animal described herein (e.g., mouse) expresses a dysfunctional endogenous MHC molecule (e.g., mouse H2-D1) or a dysfunctional endogenous MHC protein complex.

As used herein, the term “leukocytes” or “white blood cells” include T cells (CD3+), B cells (CD19+), myeloid cells (CD33+), NK cells (CD56+), granulocytes (CD66b+), and monocytes (CD14+). All leukocytes have nuclei, which distinguishes them from the anucleated red blood cells (RBCs) and platelets. CD45, also known as leukocyte common antigen (LCA), is a cell surface marker for leukocytes. Lymphocyte is a subtype of leukocyte. Lymphocytes include natural killer (NK) cells (which function in cell-mediated, cytotoxic innate immunity), T cells, and B cells. Myeloid cell is a subtype of leukocyte. Myeloid cells include monocytes and granulocytes.

In some embodiments, the genetically-modified non-human animal is a mouse. In some embodiments, the genetically-modified non-human animal is a B-NDG mouse. Details of B-NDG mice can be found, e.g., in PCT/CN2018/079365; U.S. Ser. No. 10/820,580B2, each of which is incorporated herein by reference in its entirety. In some embodiments, the genetically modified animal is a NSG mouse or NOG mouse. A detailed description of the NSG mice and NOD mice can be found, e.g., in Ishikawa et al. “Development of functional human blood and immune systems in NOD/SCID/IL2 receptor γ chainnull mice.” Blood 106.5 (2005): 1565-1573; Katano et al. “NOD-Rag2null IL-2Rγnull mice: an alternative to NOG mice for generation of humanized mice.” Experimental animals 63.3 (2014): 321-330, both of which are incorporated herein by reference in the entirety.

In one aspect, the genetically-modified non-human animal (e.g., mouse) is engrafted with human hematopoietic stem cells to develop a human immune system.

In one aspect, the genetically-modified animal is engrafted with human hematopoietic stem cells to develop a human immune system. In some embodiments, the average percentage of human leukocytes (or CD45+ cells) in the animal is at least or about 10%, 15%, 20%, 25%, 30%, 35%, 40%, 45%, or 50% of the total live cells (e.g., from blood after lysis of red blood cells) in the animal. In some embodiments, the average percentage of human leukocytes (or CD45+ cells) in the animal is at least or about 50%, 80%, 1-fold, 2-fold, 3-fold, 4-fold, 5-fold, 6-fold, 7-fold, 8-fold, 9-fold, 10-fold, 15-fold, or 20-fold higher than that of an animal with B-NDG background (e.g., a B-NDG mouse), wherein the animal with B-NDG background is irradiated and then engrafted with human hematopoietic stem cells to develop a human immune system. In some embodiments, the average percentage of human leukocytes (or CD45+ cells) is determined at least or about 12 weeks, at least or about 16 weeks, at least or about 20 weeks, at least or about 24 weeks, at least or about 26 weeks, at least or about 28 weeks, or at least or about 30 weeks after being engrafted.

In some embodiments, the success rate of reconstruction in the genetically-modified animal (e.g., mouse) is at least or about 20%, 25%, 30%, 35%, 40%, 45%, 50%, 55%, 60%, 65%, 70%, 75%, 80%, 85%, 90%, 95%, or 100%. In some embodiments, the success rate of reconstruction in the genetically-modified animal (e.g., mouse) is at least or about 10%, 20%, 30%, 40%, 50%, 60%, 70%, 80%, 90%, 1-fold, 2-fold, 3-fold, 5-fold, 10-fold, 20-fold, 50-fold, or 100-fold higher than that of an animal with B-NDG background (e.g., a B-NDG mouse). The success rate is calculated by dividing number of mice with successfully reconstructed immune system (hCD45+ cell percentage≥25% of total live cells from blood after lysis of red blood cells) over total number of survived mice. In some embodiments, the success rate is determined at least or about 16 weeks, at least or about 20 weeks, at least or about 24 weeks, at least or about 26 weeks, at least or about 28 weeks, or at least or about 30 weeks after the animal (e.g., mouse) is engrafted with human cells (e.g., hematopoietic stem cells) to develop a human immune system. In some embodiments, at least or about 16 weeks after engraftment, the success rate of reconstruction in the animal is at least or about 50%, 55%, 60%, 65%, 70%, 75%, 80%, 85%, 90%, 95%, or 100% (e.g., 80%). In some embodiments, at least or about 20 weeks after engraftment, the success rate of reconstruction in the animal is at least or about 50%, 55%, 60%, 65%, 70%, 75%, 80%, 85%, 90%, 95%, or 100% (e.g., 80%).

In some embodiments, the survival rate of the genetically-modified animal (e.g., mouse) is at least or about 20%, 25%, 30%, 35%, 40%, 45%, 50%, 55%, 60%, 65%, 70%, 75%, 80%, 85%, 90%, 95%, or 100% after about 100 days, about 120 days, about 140 days, about 160 days, or about 180 days of the engraftment. In some embodiments, the survival rate of the genetically-modified animal (e.g., mouse) is at least or about 10%, 20%, 30%, 40%, 50%, 60%, 70%, 80%, 90%, 1-fold, 2-fold, 3-fold, 5-fold, or 10-fold higher than that of an animal with B-NDG background (e.g., a B-NDG mouse), after about 100 days, about 120 days, about 140 days, about 160 days, or about 180 days of the engraftment.

The genetically modified non-human animal can also be various other animals, e.g., a rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiment, the rodent is selected from the superfamily Muroidea. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In one embodiment, the non-human animal is a mouse.

In some embodiments, the animal is a mouse of a strain selected from BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, C57BL/Ola, C57BL, C58, CBA/Br, CBA/Ca, CBA/J, CBA/st, and CBA/H. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiment, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the animal expressing human or humanized B2M and/or MHC molecule (e.g., MHC class I α chain) is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes).

Non-limiting examples of such mice include, e.g., NOD mice, SCID mice, NOD/SCID mice, nude mice, NOD/SCID nude mice, NOD-Rag 1^{-/-}-IL2rg^{-/-} (NRG) mice, Rag 2^{-/-}-IL2rg^{-/-} (RG) mice, B-NDG (NOD-Prkdc^{scid} IL-2rγ^{null}) mice, and Rag1 and/or Rag2 knockout mice. In some embodiments, these mice can optionally be irradiated, or otherwise treated to destroy one or more immune cell types. Thus, in various embodiments, a genetically modified mouse is provided that can include one or more mutations at the endogenous non-human B2M or MHC gene locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, B-NDG (NOD-Prkdc^{scid} IL-2rγ^{null}) mice, nude mice, Rag1 and/or Rag2 knockout mice, and a combination thereof. These genetically modified animals are described, e.g., in US20150106961 and PCT/CN2018/079365; each of which is incorporated herein by reference in its entirety.

Although genetically modified cells are also provided that can comprise the modifications (e.g., disruption, mutations) described herein (e.g., ES cells, somatic cells), in many embodiments, the genetically modified non-human animals comprise the modification of the endogenous B2M and/or MHC gene locus in the germline of the animal.

Furthermore, the genetically modified animal can be homozygous with respect to the modifications (e.g., replacement) of the endogenous B2M and/or MHC gene. In some embodiments, the animal can be heterozygous with respect to the modification (e.g., replacement) of the endogenous B2M and/or MHC gene.

In one aspect, the disclosure relates to a genetically-modified, non-human animal whose genome comprise a disruption in the animal's endogenous CD132 gene, wherein the disruption of the endogenous CD132 gene comprises deletion of exon 2 of the endogenous CD132 gene.

In some embodiments, the disruption of the endogenous CD132 gene further comprises deletion of exon 1 of the endogenous CD132 gene. In some embodiments, the disruption of the endogenous CD132 gene comprises deletion of part of exon 1 of the endogenous CD132 gene.

In some embodiments, the disruption of the endogenous CD132 gene further comprises deletion of one or more exons or part of exons selected from the group consisting of exon 3, exon 4, exon 5, exon 6, exon 7, and exon 8 of the endogenous CD132 gene. In some embodiments, the disruption of the endogenous CD132 gene comprises deletion of exons 1-8 of the endogenous CD132 gene.

In some embodiments, the disruption of the endogenous CD132 gene further comprises deletion of one or more introns or part of introns selected from the group consisting of intron 1, intron 2, intron 3, intron 4, intron 5, intron 6, and intron 7 of the endogenous CD132 gene.

In some embodiments, the disruption consists of deletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8.

In some embodiments, the animal is homozygous with respect to the disruption of the endogenous CD132 gene. In some embodiments, the animal is heterozygous with respect to the disruption of the endogenous CD132 gene.

In some embodiments, the disruption prevents the expression of functional CD132 protein.

In some embodiments, the length of the remaining exon sequences at the endogenous CD132 gene locus is less than 30% of the total length of all exon sequences of the endogenous CD132 gene. In some embodiments, the length of the remaining sequences at that the endogenous CD132 gene locus is less than 15% of the full sequence of the endogenous CD132 gene.

In another aspect, the disclosure relates to a genetically-modified, non-human animal, wherein the genome of the animal does not have exon 2 of CD132 gene at the animal's endogenous CD132 gene locus.

In some embodiments, the genome of the animal does not have one or more exons or part of exons selected from the group consisting of exon 1, exon 3, exon 4, exon 5, exon 6, exon 7, and exon 8. In some embodiments, the genome of the animal does not have one or more introns or part of introns selected from the group consisting of intron 1, intron 2, intron 3, intron 4, intron 5, intron 6, and intron 7.

In one aspect, the disclosure also provides a CD132 knockout non-human animal, wherein the genome of the animal comprises from 5′ to 3′ at the endogenous CD132 gene locus, (a) a first DNA sequence; optionally (b) a second DNA sequence comprising an exogenous sequence; (c) a third DNA sequence, wherein the first DNA sequence, the optional second DNA sequence, and the third DNA sequence are linked, wherein the first DNA sequence comprises an endogenous CD132 gene sequence that is located upstream of intron 1, the second DNA sequence can have a length of 0 nucleotides to 300 nucleotides, and the third DNA sequence comprises an endogenous CD132 gene sequence that is located downstream of intron 7.

In some embodiments, the first DNA sequence comprises a sequence that has a length (5′ to 3′) of from 10 to 100 nucleotides (e.g., approximately 10, 20, 30, 40, 50, 60, 70, 80, 90, 100 nucleotides), wherein the length of the sequence refers to the length from the first nucleotide in exon 1 of the CD132 gene to the last nucleotide of the first DNA sequence.

In some embodiments, the first DNA sequence comprises at least 10 nucleotides from exon 1 of the endogenous CD132 gene. In some embodiments, the first DNA sequence has at most 100 nucleotides from exon 1 of the endogenous CD132 gene.

In some embodiments, the third DNA sequence comprises a sequence that has a length (5′ to 3′) of from 200 to 600 nucleotides (e.g., approximately 200, 250, 300, 350, 400, 450, 500, 550, 600 nucleotides), wherein the length of the sequence refers to the length from the first nucleotide in the third DNA sequence to the last nucleotide in exon 8 of the endogenous CD132 gene.

In some embodiments, the third DNA sequence comprises at least 300 nucleotides from exon 8 of the endogenous CD132 gene. In some embodiments, the third DNA sequence has at most 400 nucleotides from exon 8 of the endogenous CD132 gene.

In one aspect, the disclosure also relates to a genetically-modified, non-human animal produced by a method comprising knocking out one or more exons of endogenous CD132 gene by using (1) a first nuclease comprising a zinc finger protein, a TAL-effector domain, or a single guide RNA (sgRNA) DNA-binding domain that binds to a target sequence in exon 1 of the endogenous CD132 gene or upstream of exon 1 of the endogenous CD132 gene, and (2) a second nuclease comprising a zinc finger protein, a TAL-effector domain, or a single guide RNA (sgRNA) DNA-binding domain that binds to a sequence in exon 8 of the endogenous CD132 gene. In some embodiments, the nuclease is CRISPR associated protein 9 (Cas9). In some embodiments, the animal does not express a functional CD132 protein. In some embodiments, the animal does not express a functional interleukin-2 receptor.

In one aspect, the disclosure relates to a genetically-modified mouse or a progeny thereof, whose genome comprises a disruption in the mouse's endogenous CD132 gene, wherein the disruption of the endogenous CD132 gene comprises deletion of more than 150 nucleotides in exon 1; deletion of the entirety of intron 1, exon 2, intron 2, exon 3, intron 3, exon 4, intron 4, exon 5, intron 5, exon 6, intron 6, exon 7, intron 7; and deletion of more than 250 nucleotides in exon 8. In some embodiments, the animal has an enhanced engraftment capacity of exogenous cells relative to a NSG mouse, a NOG mouse, or a NOD/scid mouse.

The present disclosure further relates to a non-human mammal generated through the methods as described herein. In some embodiments, the genome thereof contains human gene(s).

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains a modification (e.g., replacement) of the B2M and/or MHC gene in the genome of the animal.

Genetic, molecular and behavioral analyses for the non-human mammals described above can be performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the cell transfection techniques. The modification of B2M and/or MHC gene can be detected by a variety of methods.

There are also many analytical methods that can be used to detect DNA expression, including methods at the level of RNA (including the mRNA quantification approaches using reverse transcriptase polymerase chain reaction (RT-PCR) or Southern Blotting, and in situ hybridization) and methods at the protein level (including histochemistry, immunoblot analysis and in vitro binding studies). Analysis methods can be used to complete quantitative measurements. For example, transcription levels of wild-type genes and the modified sequences can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human proteins.

In some embodiments, the expression of human or humanized MHC protein complex, human or humanized B2M, human or humanized MHC gene (e.g., MHC class I α chain), and/or the fusion protein in a genetically modified animal is controllable, as by the addition of a specific inducer or repressor substance. In some embodiments, the specific inducer is selected from Tet-Off System/Tet-On System, or Tamoxifen System.

### Fusion Protein

In one aspect, the disclosure is related to a genetically-modified non-human animal expressing a fusion protein comprising, preferably from N-terminus to C-terminus:

(a) a human B2M (with or without a signal peptide);

(b) an optional linker peptide sequence; and

(c) a human MHC α chain (with or without a signal peptide).

In some embodiments, the human MHC α chain is a human HLA-A, HLA-B, or HLA-C α chain. In some embodiments, the human B2M does not have a signal peptide (e.g., amino acids 1-22 of SEQ ID NO: 4). In some embodiments, the human B2M comprises or consists of an amino acid sequence that is at least 70%, 75%, 80%, 85%, 90%, 95%, 99%, or 100% identical to amino acids 1-119, 23-119, or 21-119 of SEQ ID NO: 4. In some embodiments, the human HLA-A does not have a signal peptide (e.g., amino acids 1-24 of SEQ ID NO: 8, or amino acids 1-21 of SEQ ID NO: 59). In some embodiments, the human HLA-A has a signal peptide. In some embodiments, the human HLA-A is HLA-A*0101. In some embodiments, the human HLA-A comprises or consists of an amino acid sequence that is at least 70%, 75%, 80%, 85%, 90%, 95%, 99%, or 100% identical to amino acids 1-365, or 25-365 of SEQ ID NO: 8. In some embodiments, the human HLA-A is HLA-A2.1. In some embodiments, the human HLA-A comprises or consists of an amino acid sequence that is at least 70%, 75%, 80%, 85%, 90%, 95%, 99%, or 100% identical to amino acids 1-362, or 22-362 of SEQ ID NO: 59.

In one aspect, the disclosure is related to a genetically-modified non-human animal expressing a fusion protein comprising, preferably from N-terminus to C-terminus:

(a) a human B2M (with or without a signal peptide);

(b) a linker peptide sequence (optional); and

(c) a chimeric MHC α chain (with or without a signal peptide).

In some embodiments, the chimeric MHC α chain is a chimeric HLA-A, HLA-B, or HLA-C α chain. In some embodiments, the human B2M does not have a signal peptide (e.g., amino acids 1-22 of SEQ ID NO: 4). In some embodiments, the human B2M comprises or consists of an amino acid sequence that is at least 70%, 75%, 80%, 85%, 90%, 95%, 99%, or 100% identical to amino acids 1-119, 23-119, or 21-119 of SEQ ID NO: 4.

In some embodiments, the chimeric MHC α chain comprises a chimeric extracellular region, an endogenous transmembrane region, and an endogenous cytoplasmic region. In some embodiments, the chimeric extracellular region comprises a human HLA-A α1 domain (e.g., human HLA-A*0101 α1 domain, or human HLA-A2.1 α1 domain), a human HLA-A α2 domain (e.g., human HLA-A*0101 α2 domain, or human HLA-A2.1 α2 domain), and an endogenous MHC α chain α3 domain (e.g., mouse H2-D1 α3 domain). In some embodiments, the chimeric MHC α chain does not comprise a signal peptide (e.g., amino acids 1-24 of SEQ ID NO: 8, or amino acids 1-21 of SEQ ID NO: 59). In some embodiments, the chimeric MHC α chain comprises a human HLA-A α1 domain that is at least 70%, 75%, 80%, 85%, 90%, 95%, 99%, or 100% identical to amino acids 25-114 of SEQ ID NO: 8, or amino acids 22-111 of SEQ ID NO: 59. In some embodiments, the chimeric MHC α chain comprises a human HLA-A α2 domain that is at least 70%, 75%, 80%, 85%, 90%, 95%, 99%, or 100% identical to amino acids 115-206 of SEQ ID NO: 8, or amino acids 112-203 of SEQ ID NO: 59. In some embodiments, the chimeric MHC α chain comprises human HLA-A α1 and α2 domains that is at least 70%, 75%, 80%, 85%, 90%, 95%, 99%, or 100% identical to amino acids 25-206 or 1-206 of SEQ ID NO: 8; or amino acids 22-203 or 1-203 of SEQ ID NO: 59. In some embodiments, the chimeric MHC α chain comprises an endogenous MHC α chain α3 domain that is at least 70%, 75%, 80%, 85%, 90%, 95%, 99%, or 100% identical to amino acids 207-298 of SEQ ID NO: 6. In some embodiments, the chimeric MHC α chain further comprises an endogenous MHC α chain connecting peptide that is at least 70%, 75%, 80%, 85%, 90%, 95%, 99%, or 100% identical to amino acids 299-309 of SEQ ID NO: 6. In some embodiments, the chimeric MHC α chain comprises an endogenous MHC α chain transmembrane region that is at least 70%, 75%, 80%, 85%, 90%, 95%, 99%, or 100% identical to amino acids 310-331 of SEQ ID NO: 6. In some embodiments, the chimeric MHC α chain comprises an endogenous MHC α chain cytoplasmic region that is at least 70%, 75%, 80%, 85%, 90%, 95%, 99%, or 100% identical to amino acids 332-362 of SEQ ID NO: 6. In some embodiments, the chimeric MHC α chain comprises an endogenous MHC α chain α3 domain, an endogenous connecting peptide, an endogenous transmembrane region, and an endogenous cytoplasmic region that is at least 70%, 75%, 80%, 85%, 90%, 95%, 99%, or 100% identical to amino acids 207-362 of SEQ ID NO: 6.

In some embodiments, the fusion protein described herein is encoded by a nucleotide sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 9100, 92%, 93%, 94%, 95%, 96%, 97%, 98%, 99% of the sequence are identical to or derived from human B2M mRNA sequence (e.g., NM_004048.3 (SEQ ID NO: 3)), or a portion thereof (e.g., a portion of exon 1, exon 2, and a portion of exon 3). In some embodiments, the fusion protein described herein comprises an amino acid sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the amino acid sequence are identical or derived from human B2M amino acid sequence (e.g., NP_000563.1 (SEQ ID NO: 4)), or a portion thereof (e.g., amino acids 1-119, 23-119, or 21-119 of SEQ ID NO: 4). In some embodiments, the nucleotide sequence is a cDNA sequence.

In some embodiments, the fusion protein described herein is encoded by a nucleotide sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 9100, 92%, 93%, 94%, 95%, 96%, 97%, 98%, 99% of the sequence are identical to or derived from human HLA-A DNA or mRNA sequence (e.g., nucleic acids 95493-99436 of AF055066.1 (SEQ ID NO: 54); or NM_001242758.1 (SEQ ID NO: 7)), or a portion thereof (e.g., exon 2 and exon 3 of SEQ ID NO: 7). In some embodiments, the fusion protein described herein comprises an amino acid sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the amino acid sequence are identical or derived from human HLA-A amino acid sequence (e.g., AAC24825.1 (SEQ ID NO: 59) or NP_001229687.1 (SEQ ID NO: 8)), or a portion thereof (e.g., amino acids 1-206 or 25-206 of SEQ ID NO: 8; or amino acids 1-203 or 22-203 of SEQ ID NO: 59).

In some embodiments, the fusion protein described herein is encoded by a nucleotide sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 9100, 92%, 93%, 94%, 95%, 96%, 97%, 98%, 99% of the sequence are identical to or derived from endogenous MHC α chain mRNA sequence (e.g., mouse H2-D1 mRNA sequence NM_010380.3 (SEQ ID NO: 5)), or a portion thereof (e.g., exon 4, exon 5, exon 6, exon 7, and exon 8 of SEQ ID NO: 5). In some embodiments, the fusion protein described herein comprises an amino acid sequence. In some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 9100, 92%, 93%, 94%, 95%, 96%, 97%, 98%, 99% of the amino acid sequence are identical or derived from endogenous MHC α chain amino acid sequence (e.g., mouse H2-D1 amino acid sequence NP_034510.3 (SEQ ID NO: 6)), or a portion thereof (e.g., amino acids 207-362 of SEQ ID NO: 6).

In some embodiments, the fusion protein described herein is encoded by a nucleotide sequence. In some embodiments, the nucleotide sequence which further comprises a 3′ UTR of the endogenous MHC α chain mRNA sequence (e.g., 3′UTR of mouse H2-D1 mRNA sequence NM_010380.3 (SEQ ID NO: 5)), preferably at the 3′ end of the nucleotide sequence.

In some embodiments, the genome of the animal comprises at least one chromosome comprising a sequence encoding the fusion protein. In some embodiments, the sequence encoding the fusion protein is operably linked to a promotor or regulatory element, e.g., an endogenous B2M (e.g., mouse B2M) promotor, an inducible promoter, an enhancer, and/or mouse or human regulatory elements. In some embodiments, the sequence encoding the fusion protein is operably linked to a promotor or regulatory element, e.g., an endogenous mouse H2-D1 promotor, an inducible promoter, an enhancer, and/or mouse or human regulatory elements.

In some embodiments, all or a part of endogenous B2M locus (e.g., a portion of exon 1, exon 2, and a portion of exon 3 of mouse B2M gene locus) is replaced with a sequence encoding the fusion protein. In some embodiments, the replaced sequence is the entire coding region of the endogenous B2M gene. In some embodiments, the replaced sequence encodes a region of mouse B2M (e.g., amino acids 1-119 or 23-119 of SEQ ID NO: 2). In some embodiments, all or a part of endogenous MHC α chain gene locus (e.g., a portion of exon 1, exon 2, and exon 3 of mouse H2-D1 gene locus) is replaced with a sequence encoding the fusion protein. In some embodiments, the replaced region is the entire coding region of the endogenous MHC α chain gene (e.g., mouse H2-D1 gene). In some embodiments, the replaced sequence encodes a region of mouse H2-D1 (e.g., amino acids 1-206 or 25-206 of SEQ ID NO: 6).

In some embodiments, all or a part of endogenous B2M gene is knocked out. In some embodiments, all or a part of endogenous MHC α chain gene (e.g., mouse H2-D1 gene) is knocked out.

In some embodiments, a recombinant sequence encoding the fusion protein described herein is inserted within the endogenous B2M or MHC α chain gene locus. In some embodiments, the endogenous B2M or MHC α chain gene coding region are not transcribed or translated, due to the presence of a stop codon and the polyA signal after the inserted recombinant sequence.

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire human B2M nucleotide sequence (e.g., exon 1, exon 2, exon 3, exon 4, or NM_004048.4 (SEQ ID NO: 3)).

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as part of or the entire human B2M nucleotide sequence (e.g., exon 1, exon 2, exon 3, exon 4, or NM_004048.4 (SEQ ID NO: 3)).

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire human HLA-A nucleotide sequence (e.g., exon 1, exon 2, exon 3, exon 4, exon 5, exon 6, exon 7, exon 8 of NM_001242758.1 (SEQ ID NO: 7), or nucleic acids 95493-99436 of AF055066.1 (SEQ ID NO: 54)).

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as part of or the entire human HLA-A nucleotide sequence (e.g., exon 1, exon 2, exon 3, exon 4, exon 5, exon 6, exon 7, exon 8 of NM_001242758.1 (SEQ ID NO: 7), or nucleic acids 95493-99436 of AF055066.1 (SEQ ID NO: 54)).

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse H2-D1 nucleotide sequence (e.g., exon 1, exon 2, exon 3, exon 4, exon 5, exon 6, exon 7, exon 8, or NM_010380.3 (SEQ ID NO: 5)).

In some embodiments, the nucleotide sequence encoding the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are the same as part of or the entire mouse H2-D1 nucleotide sequence (e.g., exon 1, exon 2, exon 3, exon 4, exon 5, exon 6, exon 7, exon 8, or NM_010380.3 (SEQ ID NO: 5)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are different from part of or the entire human B2M amino acid sequence (e.g., amino acids encoded by exon 1, exon 2, exon 3, exon 4, or NM_004048.4 (SEQ ID NO: 3); or NP_004039.1 (SEQ ID NO: 4)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are the same as part of or the entire human B2M amino acid sequence (e.g., amino acids encoded by exon 1, exon 2, exon 3, exon 4, or NM_004048.4 (SEQ ID NO: 3); or NP_004039.1 (SEQ ID NO: 4)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are different from part of or the entire human HLA-A amino acid sequence (e.g., amino acids encoded by exon 1, exon 2, exon 3, exon 4, exon 5, exon 6, exon 7, exon 8 of NM_001242758.1 (SEQ ID NO: 7), or nucleic acids 95493-99436 of AF055066.1 (SEQ ID NO: 54); NP_001229687.1 (SEQ ID NO: 8); or AAC24825.1 (SEQ ID NO: 59)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are the same as part of or the entire human HLA-A amino acid sequence (e.g., amino acids encoded by exon 1, exon 2, exon 3, exon 4, exon 5, exon 6, exon 7, exon 8 of NM_001242758.1 (SEQ ID NO: 7), or nucleic acids 95493-99436 of AF055066.1 (SEQ ID NO: 54); NP_001229687.1 (SEQ ID NO: 8); or AAC24825.1 (SEQ ID NO: 59)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are different from part of or the entire mouse H2-D1 amino acid sequence (e.g., amino acids encoded by exon 1, exon 2, exon 3, exon 4, exon 5, exon 6, exon 7, exon 8, or NM_010380.3 (SEQ ID NO: 5); or NP_034510.3 (SEQ ID NO: 6)).

In some embodiments, the amino acid sequence of the fusion protein described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acids, e.g., contiguous or non-contiguous nucleotides) that are the same as part of or the entire mouse H2-D1 amino acid sequence (e.g., amino acids encoded by exon 1, exon 2, exon 3, exon 4, exon 5, exon 6, exon 7, exon 8, or NM_010380.3 (SEQ ID NO: 5); or NP_034510.3 (SEQ ID NO: 6)).

In some embodiments, the fusion protein described herein comprises or consists of an amino acid sequence, wherein the amino acid sequence is selected from the group consisting of:

a) an amino acid sequence shown in SEQ ID NO: 4, 8, 59, 61, 62, 63, or 64;

b) an amino acid sequence having a homology of at least 90% with or at least 90% identical to the amino acid sequence shown in SEQ ID NO: 4, 8, 59, 61, 62, 63, or 64;

c) an amino acid sequence encoded by a nucleic acid sequence, wherein the nucleic acid sequence is able to hybridize to a nucleotide sequence encoding the amino acid shown in SEQ ID NO: 4, 8, 59, 61, 62, 63, or 64 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 4, 8, 59, 61, 62, 63, or 64;

e) an amino acid sequence that is different from the amino acid sequence shown in SEQ ID NO: 4, 8, 59, 61, 62, 63, or 64 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 4, 8, 59, 61, 62, 63, or 64.

The present disclosure also relates to a nucleic acid (e.g., DNA or RNA) sequence, wherein the nucleic acid sequence can be selected from the group consisting of:

a) a nucleic acid sequence as shown in SEQ ID NO: 9, 10, 13, 14, 15, 16, 52, 54, or 65, or a nucleic acid sequence encoding a homologous B2M or MHC α chain amino acid sequence of a humanized mouse B2M or MHC α chain;

b) a nucleic acid sequence that is able to hybridize to the nucleotide sequence as shown in SEQ ID NO: 9, 10, 13, 14, 15, 16, 52, 54, or 65 under a low stringency condition or a strict stringency condition;

c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 9, 10, 13, 14, 15, 16, 52, 54, or 65;

d) a nucleic acid sequence that encodes an amino acid sequence, wherein the amino acid sequence has a homology of at least 90% with or at least 90% identical to the amino acid sequence shown in SEQ ID NO: 4, 8, 59, 61, 62, 63, or 64;

e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 4, 8, 59, 61, 62, 63, or 64;

f) a nucleic acid sequence that encodes an amino acid sequence, wherein the amino acid sequence is different from the amino acid sequence shown in SEQ ID NO: 4, 8, 59, 61, 62, 63, or 64 by no more than 10, 9, 8, 7, 6, 5, 4, 3, 2 or no more than 1 amino acid; and/or

g) a nucleic acid sequence that encodes an amino acid sequence, wherein the amino acid sequence comprises a substitution, a deletion and/or insertion of one or more amino acids to the amino acid sequence shown in SEQ ID NO: 4, 8, 59, 61, 62, 63, or 64.

In some embodiments, the fusion protein comprises a human MHC α chain signal peptide at the N-terminus of the fusion protein. In some embodiments, the human MHC α chain signal peptide is a signal peptide of human HLA-A (e.g., HLA-A*0101, or HLA-A2.1). In some embodiments, the signal peptide of human HLA-A comprises or consists of an amino acid sequence that is at least 70%, 75%, 80%, 85%, 90%, 95%, 99%, or 100% identical to amino acids 1-24 of SEQ ID NO: 8, or amino acids 1-21 of SEQ ID NO: 59. In some embodiments, the signal peptide of human HLA-A is encoded by a sequence that is at least 70%, 75%, 80%, 85%, 90%, 95%, 99%, or 100% identical to SEQ ID NO: 13.

In some embodiments, the fusion protein comprises an endogenous MHC molecule (e.g., mouse H2-D1 gene) signal peptide at the N-terminus of the fusion protein. In some embodiments, the signal peptide comprises or consists of an amino acid sequence that is at least 70%, 75%, 80%, 85%, 90%, 95%, 99%, or 100% identical to amino acids 1-24 of SEQ ID NO: 6.

In some embodiments, the fusion protein comprises a human or endogenous B2M signal peptide at the N-terminus of the fusion protein. In some embodiments, the human or endogenous B2M signal peptide comprises or consists of an amino acid sequence that is at least 70%, 75%, 80%, 85%, 90%, 95%, 99%, or 100% identical to amino acids 1-20 of SEQ ID NO: 4 or amino acids 1-20 of SEQ ID NO: 2.

In some embodiments, the human B2M is fused to the human MHC α chain with or without a linker peptide sequence. In some embodiments, the linker peptide sequence is optional, i.e., the two regions that are linked together can be directly linked by a peptide bond. In some embodiments, the linker peptide sequence comprises at least or about 5, 6, 7, 8, 9, 10, 11, 12, 13, 14, 15, 16, 17, 18, 19, 20, 30, 40, or 50 amino acid residues. In some embodiments, the linker peptide sequence comprises at least or about 4, 5, 6, 7, 8, 9, 10, 11, 12, 13, 14, 15, 16, 20, 25, 30, or 40 glycine residues. In some embodiments, the linker peptide sequence comprises at least or about 1, 2, 3, 4, 5, 6, 7, or 8 serine residues. In some embodiments, the linker peptide sequence comprises or consists of both glycine and serine residues. In some embodiments, the linker peptide sequence comprises or consists of a sequence that is at least or about 70%, at least or about 75%, at least or about 80%, at least or about 85%, at least or about 90%, at least or about 95%, at least or about 99%, or 100% identical to any SEQ ID NO: 67. In some embodiments, the linker peptide sequence comprises at least 1, 2, 3, 4, 5, 6, 7, or 8 repeats of GGGGS (SEQ ID NO: 68). In some embodiments, the linker peptide sequence has no more than 10, 11, 12, 13, 14, 15, 16, 17, 18, 19, 20, 30, 40, or 50 amino acid residues. In some embodiments, the linker peptide sequence is encoded by a sequence that is at least 70%, 75%, 80%, 85%, 90%, 95%, 99%, or 100% identical to SEQ ID NO: 51.

In some embodiments, the genetically-modified non-human animal expressing a humanized MHC protein complex described herein expresses normal level of endogenous B2M (e.g., mouse B2M). In some embodiments, the genetically-modified non-human animal expressing a humanized MHC protein complex described herein expressed a decreased level (e.g., less than 90%, less than 80%, less than 70%, less than 60%, or less than 50% as compared to that of an animal without the genetic modification) of endogenous B2M (e.g., mouse B2M). In some embodiments, the genetically-modified non-human animal expressing a humanized MHC protein complex described herein does not express endogenous B2M (e.g., mouse B2M).

### Vectors

The disclosure also provides vectors for constructing a humanized MHC protein complex animal model. In some embodiments, the vectors comprise a sgRNA sequence. In some embodiments, the sgRNA sequence targets B2M gene (e.g., of the non-human animal described herein), and the sgRNA is unique on the target sequence of the B2M gene to be altered, and meets the sequence arrangement rule of 5′-NNN (20)-NGG3′ or 5′-CCN-N (20)-3′. In some embodiments, the targeting site of the sgRNA in the mouse B2M gene is located on the exon 1, exon 2, exon 3, exon 4, intron 1, intron 2, intron 3, upstream of exon 1, or downstream of exon 4 of the mouse B2M gene. In some embodiments, the targeting site of the sgRNA in the mouse B2M gene is located on exon 1 or intron 1. In some embodiments, the targeting site of the sgRNA in the mouse B2M gene is located on exon 3 or intron 3.

In some embodiments, the sgRNA sequence recognizes a targeting site within exon 1 or intron 1 of mouse B2M gene. In some embodiments, the targeting sites within exon 1 or intron 1 are set forth in SEQ ID NOS: 17-23. In some embodiments, the targeting site within exon 1 or intron 1 is set forth in SEQ ID NO: 19. In some embodiments, the sgRNA sequence recognizes a targeting site within exon 3 or intron 3 of mouse B2M gene. In some embodiments, the targeting sites within exon 3 or intron 3 are set forth in SEQ ID NOS: 24-31. In some embodiments, the targeting site within exon 3 or intron 3 is set forth in SEQ ID NO: 30. In some embodiments, the sgRNA sequences are encoded by double-strand DNA molecules with sequences set forth in SEQ ID NO: 32 and SEQ ID NO: 34; SEQ ID NO: 33 and SEQ ID NO: 35; SEQ ID NO: 36 and SEQ ID NO: 38; or SEQ ID NO: 37 and SEQ ID NO: 39.

In some embodiments, the disclosure relates to a plasmid construct (e.g., pT7-sgRNA) including the sgRNA sequence, and/or a cell including the construct.

In some embodiments, the disclosure relates to a targeting vector including a 5′ homologous arm and a 3′ homologous arm. In some embodiments, the 5′ homologous arm comprises a sequence spanning the entire or part of upstream of exon 1, and exon 1. In some embodiments, the 3′ homologous arm comprises a sequence spanning the entire or part of intron 3, exon 4, and downstream of exon 4.

In some embodiments, the 5′ homologous arm comprises a sequence that is at least 80%, 85%, 90%, 95%, or 100% identical to SEQ ID NO: 11. In some embodiments, the 3′ homologous arm comprises a sequence that is at least 80%, 85%, 90%, 95%, or 100% identical to SEQ ID NO: 12. In some embodiments, the 5′ homologous arm comprises a sequence that is at least 80%, 85%, 90%, 95%, or 100% identical to 122146329-122147737 of the NCBI Reference Sequence NC_000068.7. In some embodiments, the 3′ homologous arm comprises a sequence that is at least 80%, 85%, 90%, 95%, 97.5%, or 100% identical to 122152171-122153513 of the NCBI Reference Sequence NC_000068.7.

In some embodiments, the targeting vector further comprises a nucleotide sequence between the 5′ and 3′ homologous arms. In some embodiments, the nucleotide sequence comprises a sequence (e.g., a cDNA sequence) encoding the entire or a part of the fusion protein described herein. In some embodiments, the nucleotide sequence comprises or consists, preferably from 5′ end to 3′ end: a sequence encoding human HLA-A2.1 signal peptide, a sequence encoding human B2M, a sequence encoding the linker peptide sequence described herein, a sequence encoding a portion of human HLA-A2.1, and a sequence encoding a portion of mouse H2-D1. In some embodiments, the sequence encoding human HLA-A2.1 signal peptide is at least 80%, 85%, 90%, 95%, 97.5%, or 100% identical to SEQ ID NO: 13. In some embodiments, the sequence encoding human B2M is at least 80%, 85%, 90%, 95%, 97.5%, or 100% identical to SEQ ID NO: 14. In some embodiments, the sequence encoding the linker peptide sequence is at least 80%, 85%, 90%, 95%, 97.5%, or 100% identical to SEQ ID NO: 51. In some embodiments, the sequence encoding the portion of human HLA-A2.1 is at least 80%, 85%, 90%, 95%, 97.5%, or 100% identical to SEQ ID NO: 15. In some embodiments, the sequence encoding the portion of mouse H2-D1 is at least 80%, 85%, 90%, 95%, 97.5%, or 100% identical to SEQ ID NO: 16.

In some embodiments, the 5′ homologous arm comprises a sequence that is at least 80%, 85%, 90%, 95%, or 100% identical to SEQ ID NO: 60. In some embodiments, the 3′ homologous arm comprises a sequence that is at least 80%, 85%, 90%, 95%, or 100% identical to SEQ ID NO: 53. In some embodiments, the 5′ homologous arm comprises a sequence that is at least 80%, 85%, 90%, 95%, 99%, or 100% identical to 122146329-122147737 of the NCBI Reference Sequence NC_000068.7. In some embodiments, the 5′ homologous arm comprises a G to T mutation at position 122147015, a C to T mutation at positions 122147108 and 122147591 of NCBI Reference Sequence NC_000068.7. In some embodiments, the 3′ homologous arm comprises a sequence that is at least 80%, 85%, 90%, 95%, 97.5%, 99%, or 100% identical to 122152171-122153513 of the NCBI Reference Sequence NC_000068.7. In some embodiments, the 3′ homologous arm comprises the following mutations within NCBI Reference Sequence NC_000068.7: mutations at position 122152258 (from G to A), position 122152391 (from G to A), position 122152771 (from A to G), position 122153104 (from T to C), and position 122153148 (from A to C); and deletion at position 122152788 (deletion of A).

In some embodiments, the targeting vector further comprises a nucleotide sequence between the 5′ and 3′ homologous arms. In some embodiments, the nucleotide sequence comprises a sequence (e.g., a cDNA sequence) encoding the entire or a part of the fusion protein described herein. In some embodiments, the nucleotide sequence comprises or consists, preferably from 5′ end to 3′ end: a sequence encoding human HLA-A2.1 signal peptide, a sequence encoding human B2M, a sequence encoding the linker peptide sequence described herein, and a sequence encoding human HLA-A2.1. In some embodiments, the sequence encoding human HLA-A2.1 signal peptide is at least 80%, 85%, 90%, 95%, 97.5%, or 100% identical to SEQ ID NO: 13. In some embodiments, the sequence encoding human B2M is at least 80%, 85%, 90%, 95%, 97.5%, or 100% identical to SEQ ID NO: 14. In some embodiments, the sequence encoding the linker peptide sequence is at least 80%, 85%, 90%, 95%, 97.5%, or 100% identical to SEQ ID NO: 51. In some embodiments, the sequence encoding human HLA-A2.1 is at least 80%, 85%, 90%, 95%, 97.5%, or 100% identical to SEQ ID NO: 54. In some embodiments, the nucleotide sequence between the 5′ and 3′ homologous arms is at least 80%, 85%, 90%, 95%, 97.5%, or 100% identical to SEQ ID NO: 65.

In addition, the present disclosure further relates to a non-human mammalian cell, having any one of the foregoing targeting vectors, and one or more in vitro transcripts of the sgRNA construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

In some embodiments, provided herein is a method for preparing a vector comprising an sgRNA sequence, the method includes the following steps: (a) providing the sgRNA sequence, which is obtained using a forward oligonucleotide sequence and a reverse oligonucleotide sequence, wherein the sgRNA sequence targets the non-human animal B2M gene described herein, wherein the sgRNA is unique on the target B2M gene to be altered, and meets the sequence arrangement rule of 5′-NNN(20)-NGG3′ or 5′-CCN-N(20)-3′; (b) synthesizing a DNA fragment containing the T7 promoter and an sgRNA scaffold (e.g., at least 80% identical to SEQ ID NO: 40), then ligating the DNA fragment to the backbone vector after EcoRI and BamHI digestion, and obtaining a pT7-sgRNA vector after verification by sequencing; (c) denaturing and annealing the forward oligonucleotide and the reverse oligonucleotide obtained in step (a) to form a double strand that can be ligated to the pT7-sgRNA vector described in step (b); (d) ligating the double-stranded sgRNA oligonucleotides annealed in step (c) with the pT7-sgRNA vector, and screening to obtain the sgRNA vector.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous B2M or MHC gene locus, a sequence encoding a region of an endogenous B2M or MHC α chain with a sequence encoding a fusion protein described herein. In some embodiments, the replacement occurs in a germ cell, a somatic cell, a blastocyst, or a fibroblast, etc. The nucleus of a somatic cell or the fibroblast can be inserted into an enucleated oocyte.

FIG. 9 and FIG. 18 shows a MHC protein complex humanization strategy for at mouse B2M gene locus. In FIG. 9 and FIG. 18, the targeting strategy involves a vector comprising the 5′ homologous arm, a sequence encoding the fusion protein, and the 3′ homologous arm. The process can involve replacing endogenous B2M gene sequence with the sequence encoding the fusion protein by homologous recombination. In some embodiments, the cleavage at the upstream and the downstream of the target site (e.g., by zinc finger nucleases, TALEN or CRISPR) can result in DNA double strands break, and the homologous recombination is used to replace endogenous B2M gene sequence with the sequence encoding the fusion protein.

In some embodiments, the sequence between the 5′ end targeting site and the 3′ end targeting site is knocked out. In some embodiments, the sequence between the 5′ end targeting site and the 3′ end targeting site is replaced. In some embodiments, the replaced sequence starts from within exon 1 or intron 1 of mouse B2M gene. In some embodiments, the replaced sequence ends within exon 3 or intron 3 of mouse B2M gene.

Thus, in some embodiments, the methods for making a genetically modified, humanized animal, can include the step of replacing at an endogenous B2M locus (or site), a nucleic acid encoding a sequence encoding a region of endogenous B2M with a sequence encoding the fusion protein described herein. The sequence can include a region (e.g., a part or the entire region) of exon 1, exon 2, exon 3, exon 4 of a an endogenous B2M gene. In some embodiments, the sequence encoding the fusion protein includes a region (e.g., a part or the entire region) of exon 1, exon 2, exon 3, exon 4 of a human B2M gene, and a region (e.g., a part or the entire region) of exon 1, exon 2, exon 3, exon 4, exon 5, exon 6, exon 7, and exon 8 of an endogenous or human MHC α chain gene. In some embodiments, the endogenous B2M locus is a portion of exon 1, exon 2, and a portion of exon 3 of mouse B2M gene (e.g., a sequence encoding amino acids 1-119 of SEQ ID NO: 2).

In some embodiments, the methods of modifying a B2M gene locus of a mouse to express the fusion protein described herein can include the steps of replacing at the endogenous mouse B2M gene locus a nucleotide sequence encoding a mouse B2M with a nucleotide sequence encoding the fusion protein, thereby generating a sequence encoding a fusion protein comprising a human B2M and a human or chimeric MHC α chain.

In some embodiments, the nucleotide sequences as described herein do not overlap with each other (e.g., the 5′ homologous arm, the A fragment (or the BNDG-A fragment), and/or the 3′ homologous arm do not overlap). In some embodiments, the amino acid sequences as described herein do not overlap with each other.

Zinc finger proteins, TAL-effector domains, or single guide RNA (sgRNA) DNA-binding domains can be designed to target regions within exon 1, exon 2, exon 3, exon 4, intron 1, intron 2, and/or intron 3 of endogenous (e.g., mouse) B2M gene locus. For example, targeting sequences of SEQ ID NOs: 17-23 are located in exon 1 or intron 1 of the endogenous (e.g., mouse) B2M gene locus; and targeting sequences of SEQ ID NOs: 24-31 are located in exon 3 or intron 3 of the endogenous (e.g., mouse) B2M gene locus. After the zinc finger proteins, TAL-effector domains, or single guide RNA (sgRNA) DNA-binding domains bind to the target sequences, the nuclease cleaves the genomic DNA. In some embodiments, the nuclease is CRISPR associated protein 9 (Cas9).

Thus, the methods of producing a mouse expressing a human or humanized MHC protein complex, human or humanized B2M, and/or human or humanized MHC molecules can involve one or more of the following steps: transforming a mouse embryonic stem cell with a gene editing system that targets endogenous B2M or MHC gene, thereby producing a transformed embryonic stem cell; introducing the transformed embryonic stem cell into a mouse blastocyst; implanting the mouse blastocyst into a pseudopregnant female mouse; and allowing the blastocyst to undergo fetal development to term.

In some embodiments, the transformed embryonic cell is directly implanted into a pseudopregnant female mouse instead, and the embryonic cell undergoes fetal development.

In some embodiments, the gene editing system can involve Zinc finger proteins, TAL-effector domains, or single guide RNA (sgRNA) DNA-binding domains.

The present disclosure further provides a method for establishing an animal model expressing a human or humanized MHC protein complex, human or humanized B2M, and/or human or humanized MHC molecules, involving the following steps:

(a) providing the cell (e.g. a fertilized egg cell) with the genetic modification based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse, a NOD/scid mouse, a NOD/scid nude mouse, or a B-NDG mouse). In some embodiments, the non-human mammal is a B-NDG (NOD-Prkdc^{scid} IL-2rγ^{null}) mouse. In some embodiments, the non-human mammal is a NOD/scid mouse.

In the B-NDG mouse, the Prkdc^{scid} (commonly known as “SCID” or “severe combined immunodeficiency”) mutation has been transferred onto a non-obese diabetic (NOD) background. Animals homozygous for the SCID mutation have impaired T and B cell lymphocyte development. The NOD background additionally results in deficient natural killer (NK) cell function. IL-2rγ^{null} refers to a specific knock out modification in mouse CD132 gene. Details can be found, e.g., in PCT/CN2018/079365, which is incorporated herein by reference in its entirety. In some embodiments, the non-human mammal is a B-NDG mouse. The B-NDG mouse additionally has a disruption of FOXN1 gene on chromosome 11 in mice.

In some embodiments, the fertilized eggs for the methods described above are NOD/scid fertilized eggs, NOD/scid nude fertilized eggs, or B-NDG fertilized eggs. Other fertilized eggs that can also be used in the methods as described herein include, but are not limited to, C57BL/6 fertilized eggs, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the method described above.

The genetically modified animals (e.g., mice) as described herein can have several advantages. For example, the genetically modified mice do not require backcrossing, and thus have a relatively purer background (e.g., B-NDG) as compared to some other immunodeficient mice known in the art. A pure background is beneficial to obtain consistent experiment results.

### Methods of Using Genetically Modified Animals

Genetically modified animals that express a human or humanized MHC protein complex can provide a variety of uses that include, but are not limited to, establishing a human hemato-lymphoid animal model, developing therapeutics for human diseases and disorders, and assessing the efficacy of these therapeutics in the animal models.

In some embodiments, the genetically modified animals can be used for establishing a human hemato-lymphoid system. The methods involve engrafting a population of cells comprising human hematopoietic cells (CD34+ cells) or human peripheral blood cells into the genetically modified animal described herein. In some embodiments, the methods further include the step of irradiating the animal prior to the engrafting. In some embodiments, the step of irradiating is not required prior to the engrafting. The human hemato-lymphoid system in the genetically modified animals can include various human cells, e.g., hematopoietic stem cells, myeloid precursor cells, myeloid cells, dendritic cells, monocytes, granulocytes, neutrophils, mast cells, lymphocytes, and platelets.

The genetically modified animals described herein (e.g., expressing human or humanized MHC protein complex) are also an excellent animal model for establishing the human hemato-lymphoid system. In some embodiments, the animal after being engrafted with human hematopoietic stem cells or human peripheral blood cells to develop a human immune system has one or more of the following characteristics:
(a) the percentage of human leukocytes (or CD45+ cells) is at least or about 10%, 15%, 20%, 25%, 30%, 35%, 40%, 45%, or 50% of total live cells from blood (after lysis of red blood cells) in the animal;
(b) the percentage of human T cells (or CD3+ cells) is at least or about 1%, 2%, 3%, 4%, 5%, 8%, 10%, 15%, 20%, 30%, 40%, or 50% of human leukocytes (or CD45+ cells) in the animal;
(c) the percentage of human B cells (or CD19+ cells) is at least or about 30%, 35%, 40%, 45%, 50%, 55%, 60%, 65%, 70%, 75%, or 80% of human leukocytes (or CD45+ cells) in the animal;
(d) the percentage of human NK cells (or CD56+ cells) is at least or about 1%, 2%, 3%, 4%, 5%, 8%, or 10% of human leukocytes (or CD45+ cells) in the animal
(e) the percentage of human myeloid cells (or CD33+ cells) is at least or about 2%, 5%, 8%, 10%, 15%, or 20% of human leukocytes (or CD45+ cells) in the animal;
(f) the percentage of human monocytes (or CD14+ cells) is at least or about 50%, 55%, 60%, 65%, 70%, 75%, 80%, 85%, 90%, or 95% of human myeloid cells (or CD33+ cells) in the animal; and
(g) the percentage of human granulocytes (or CD66b+ cells) is at least or about 1%, 2%, 3%, 4%, 5%, 8%, 10%, 15%, 20%, 25%, or 30% of human myeloid cells (or CD33+ cells) in the animal.

In some embodiments, the one or more characteristics are determined at least or about 4 weeks, at least or about 8 weeks, at least or about 12 weeks, at least or about 16 weeks, at least or about 20 weeks, at least or about 24 weeks, at least or about 26 weeks, at least or about 28 weeks, at least or about 30 weeks after the animal (mouse) is engrafted with human hematopoietic stem cells to develop a human immune system.

In some embodiments, the animal has an enhanced engraftment capacity of exogenous cells relative to a NSG mouse, a NOG mouse, a NOD/scid mouse, or a B-NDG mouse. In some embodiments, the animal models described here are better animal models for establishing the human hemato-lymphoid system (e.g. having a higher survival rate; having a higher percentage of leukocytes in total live cells; or having a higher success rate of reconstruction). A detailed description of the NSG mice, NOD mice, and B-NDG can be found, e.g., in Ishikawa et al. “Development of functional human blood and immune systems in NOD/SCID/IL2 receptor γ chainnull mice.” Blood 106.5 (2005): 1565-1573; Katano et al. “NOD-Rag2null IL-2Rγnull mice: an alternative to NOG mice for generation of humanized mice.” Experimental animals 63.3 (2014): 321-330; US20190320631A1; each of which is incorporated herein by reference in the entirety.

In some embodiments, the genetically modified animals can be used to determine the effectiveness of an agent or a combination of agents for the treatment of cancer. The methods involve engrafting tumor cells to the animal as described herein, administering the agent or the combination of agents to the animal; and determining the inhibitory effects on the tumors.

In some embodiments, the tumor cells are from a tumor sample obtained from a human patient. These animal models are also known as Patient derived xenografts (PDX) models. PDX models are often used to create an environment that resembles the natural growth of cancer, for the study of cancer progression and treatment. Within PDX models, patient tumor samples grow in physiologically-relevant tumor microenvironments that mimic the oxygen, nutrient, and hormone levels that are found in the patient's primary tumor site. Furthermore, implanted tumor tissue maintains the genetic and epigenetic abnormalities found in the patient and the xenograft tissue can be excised from the patient to include the surrounding human stroma. As a result, PDX models can often exhibit similar responses to anti-cancer agents as seen in the actual patient who provide the tumor sample.

While the genetically modified immunodeficient animals (e.g., mice with B-NDG background) do not have functional T cells or B cells, the animals still have functional phagocytic cells, e.g., neutrophils, eosinophils (acidophilus), basophils, or monocytes. Macrophages can be derived from monocytes, and can engulf and digest cellular debris, foreign substances, microbes, cancer cells. Thus, the genetically modified animals described herein can be used to determine the effect of an agent (e.g., anti-CD47 antibodies, anti-IL6 antibodies, anti-IL15 antibodies, or anti-SIRPα antibodies) on phagocytosis, and the effects of the agent to inhibit the growth of tumor cells.

In some embodiments, human peripheral blood cells (hPBMC) or human hematopoietic stem cells are injected to the animal to develop human hematopoietic system. The genetically modified animals described herein can be used to determine the effect of an agent in human hematopoietic system, and the effects of the agent to inhibit tumor cell growth or tumor growth. Thus, in some embodiments, the methods as described herein are also designed to determine the effects of the agent on human immune cells (e.g., human T cells, B cells, or NK cells), e.g., whether the agent can stimulate T cells or inhibit T cells, whether the agent can upregulate the immune response or downregulate immune response. In some embodiments, the genetically modified animals can be used for determining the effective dosage of a therapeutic agent for treating a disease in the subject, e.g., cancer, or autoimmune diseases.

In some embodiments, the tested agent or the combination of tested agents is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the tested agent is designed for the treating melanoma, primary lung carcinoma, non-small cell lung carcinoma (NSCLC), small cell lung cancer (SCLC), primary gastric carcinoma, bladder cancer, breast cancer, and/or prostate cancer.

In some embodiments, the injected tumor cells are human tumor cells. In some embodiments, the injected tumor cells are melanoma cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

The inhibitory effects on tumors can also be determined by any methods known in the art. In some embodiments, the tumor cells can be labeled by a luciferase gene. Thus, the number of the tumor cells or the size of the tumor in the animal can be determined by an in vivo imaging system (e.g., the intensity of fluorescence). In some embodiments, the inhibitory effects on tumors can also be determined by measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the tested agent can be one or more agents selected from the group consisting of paclitaxel, cisplatin, carboplatin, pemetrexed, 5-FU, gemcitabine, oxaliplatin, docetaxel, and capecitabine.

In some embodiments, the tested agent can be an antibody, for example, an antibody that binds to CSF2, IL3, CSF1, IL15, CD47, PD-1, CTLA-4, LAG-3, TIM-3, BTLA, PD-L1, 4-1BB, CD27, CD28, CD47, TIGIT, CD27, GITR, or OX40. In some embodiments, the antibody is a human antibody.

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

In most immunodeficient mice (e.g., B-NDG mice), the MHC protein complex is mouse endogenous MHC protein complex (e.g., comprising mouse H2 molecules). After transplantation of human cells or tissues, human-derived cells (e.g., human hematopoietic cells or human peripheral blood cells) cannot obtain human MHC-restricted antigen recognition. In addition, human MHC-restricted immune response cannot be evaluated after immunotherapy or infection by specific pathogens in the immunodeficient mice. Further, transplanted human T and B lymphocytes cannot be fully functionally mature in the immunodeficient mice. In some embodiments, the genetically-modified non-human animal expressing a humanized MHC protein complex described herein can improve MHC restriction effect after being engrafted with human cells (e.g., human hematopoietic cells or human peripheral blood cells) or tissues by at least 10%, 20%, 30%, 40%, 50%, 60%, 70%, 80%, 90%, or 100%, as compared to that in control mice (with the same background) without expressing the humanized MHC protein complex. In some embodiments, the genetically-modified non-human animal expressing a humanized MHC protein complex described herein can improve human T cells and/or B cells maturation after being engrafted with human cells (e.g., human hematopoietic cells or human peripheral blood cells) or tissues by at least 10%, 20%, 30%, 40%, 50%, 60%, 70%, 80%, 90%, or 100%, as compared to that in control mice (with the same background) without expressing the humanized MHC protein complex.

Thus, in one aspect, the genetic modified animals as described herein are particularly suitable for evaluating the efficacy of cell therapy (e.g., T cell based cell therapy). In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying immunotherapies (e.g., human CAR-T therapy) to the animal with human tumor cells. Effectiveness of the CAR-T therapy can be determined and evaluated. In some embodiments, the animal is selected from the non-human animal prepared by the methods described herein, the non-human animal described herein, the double- or multi-humanized non-human animal generated by the methods described herein (or progeny thereof), a non-human animal expressing humanized MHIC protein complex, or the tumor-bearing or inflammatory animal models described herein. In some embodiments, the TCR-T, CAR-T, and/or other immunotherapies can treat the diseases described herein. In some embodiments, the TCR-T, CAR-T, and/or other immunotherapies provides an evaluation method for treating the diseases (e.g., cancer) described herein.

Animal Models with Additional Genetic Modifications

The present disclosure further relates to methods for generating genetically modified animal models described herein with some additional modifications (e.g., human or chimeric genes or additional gene knockout).

In some embodiments, the animal can comprise a sequence encoding a human or humanized MHIC protein complex and a sequence encoding an additional human or chimeric protein. In some embodiments, the additional human or chimeric protein can be Colony Stimulating Factor 2 (CSF2), IL3, Colony Stimulating Factor 1 (CSF1), IL15, programmed cell death protein 1 (PD-1), TNF Receptor Superfamily Member 9 (4-1BB or CD137), cytotoxic T-lymphocyte-associated protein 4 (CTLA-4), LAG-3, T-cell immunoglobulin and mucin-domain containing-3 (TIM-3), B And T Lymphocyte Associated (BTLA), Programmed Cell Death 1 Ligand 1 (PD-L1), CD27, CD28, Signal-regulatory protein alpha (SIRPα), CD47, Thrombopoietin (THPO), T-Cell Immunoreceptor With Ig And ITIM Domains (TIGIT), Glucocorticoid-Induced TNFR-Related Protein (GITR), or TNF Receptor Superfamily Member 4 (TNFRSF4; or OX40).

In some embodiments, the animal can comprise a sequence encoding a human or humanized MHC protein complex and a disruption at some other endogenous genes (e.g., CD132, Beta-2-Microglobulin (B2m) or Forkhead Box N1 (Foxn1)). In some embodiments, the animal has a mutation in KIT. The genetically modified non-human animals with a mutation in KIT is described, e.g., in PCT/CN2020/113608, which is incorporated herein by reference in its entirety.

The methods of generating genetically modified animal model with two or more human or chimeric genes (e.g., humanized genes) can include the following steps:

(a) using the methods of introducing a sequence encoding the fusion protein as described herein to obtain a genetically modified non-human animal;

(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric CSF2, IL3, CSF1, IL15, PD-1, CTLA-4, LAG-3, TIM-3, BTLA, PD-L1, 4-1BB, CD27, CD28, SIRPα, CD47, THPO, TIGIT, GITR, or OX40. Some of these genetically modified non-human animals are described, e.g., in PCT/CN2017/090320, PCT/CN2017/099577, PCT/CN2017/099575, PCT/CN2017/099576, PCT/CN2017/099574, PCT/CN2017/106024, PCT/CN2020/125489, PCT/CN2020/142546, CN111172190A, CN111118019A, and CN111073907A; each of which is incorporated herein by reference in its entirety.

In some embodiments, the genetic modification described herein can be directly performed on a genetically modified animal having a human or chimeric CSF2, IL3, CSF1, IL15, PD-1, CTLA-4, LAG-3, BTLA, TIM-3, PD-L1, 4-1BB, CD27, CD28, SIRPα, CD47, THPO, TIGIT, GITR, or OX40 gene.

In some embodiments, the genetic modification described herein can be directly performed on a B2m knockout mouse or a Foxn1 knockout mouse. In some embodiments, the genetic modification described herein can be directly performed on a B-NDG mouse.

As these proteins may involve different mechanisms, a combination therapy that targets two or more of these proteins thereof may be a more effective treatment. In fact, many related clinical trials are in progress and have shown a good effect.

The MHC protein complex humanized animal model, and/or the MHC protein complex humanized animal model with additional genetic modifications can be used for determining effectiveness of a combination therapy.

In some embodiments, the combination of agents can include one or more agents selected from the group consisting of paclitaxel, cisplatin, carboplatin, pemetrexed, 5-FU, gemcitabine, oxaliplatin, docetaxel, and capecitabine.

In some embodiments, the combination of agents can include one or more agents selected from the group consisting of campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and methotrexate.

In some embodiments, the combination of agents can include one or more antibodies that bind to CSF2, IL3, CSF1, IL15, PD-1, CTLA-4, LAG-3, BTLA, TIM-3, PD-L1, 4-1BB, CD27, CD28, SIRPα, CD47, THPO, TIGIT, GITR, and/or OX40.

Alternatively or in addition, the methods can also include performing surgery on the subject to remove at least a portion of the cancer, e.g., to remove a portion of or all of a tumor(s), from the subject.

### DESCRIPTION OF DRAWINGS

FIG. 1 is a 3D schematic structure of HLA-A.

FIG. 2 are schematic diagrams showing mouse B2M gene locus and human B2M gene locus.

FIG. 3 are schematic diagrams showing mouse H2-D1 gene locus and human HLA-A gene locus.

FIG. 4 is a schematic diagram showing humanized mouse B2M gene locus. Mouse B2M gene coding region is replaced with a nucleic acid sequence encoding human B2M protein, a portion of human HLA-A2.1 protein, and a portion of mouse H2-D1 protein.

FIG. 5 is a schematic diagram showing humanized mouse B2M gene locus. Mouse B2M gene coding region is replaced with the coding region of human B2M gene.

FIG. 6 is a schematic diagram showing humanized mouse H2-D1 gene locus. A portion of mouse H2-D1 gene is replaced with a nucleic acid sequence encoding a portion of human HLA-A2.1 protein.

FIG. 7 is a schematic diagram showing humanized mouse H2-D1 gene locus. A portion of mouse H2-D1 gene is replaced with a nucleic acid sequence encoding human B2M protein and a portion of human HLA-A2.1 protein.

FIG. 8 is a schematic diagram showing humanized mouse B2M gene locus. Mouse B2M gene coding region was replaced with a nucleic acid sequence encoding the signal peptide of human HLA-A2.1, human B2M protein, a portion of human HLA-A2.1 protein, and a portion of mouse H2-D1 protein.

FIG. 9 shows a schematic diagram of a targeting strategy at mouse B2M gene locus.

FIG. 10A shows activity testing results for sgRNA1-sgRNA7 (sg1-sg7). PC is positive control. Con. is negative control. Blank is blank control.

FIG. 10B shows activity testing results for sgRNA9-sgRNA15 (sg9-sg15). PC is positive control. Con. is negative control. Blank is blank control.

FIG. 11A shows 5′ end PCR detection result of F0 generation mice by primers L-GT-F and L-GT-R. M is marker. H₂O is water control. WT is wildtype control. PC is positive control. F0-01, F0-02, F0-03, F0-04, F0-05, F0-06, F0-07, F0-08, F0-09, and F0-10 are mouse numbers.

FIG. 11B shows 3′ end PCR detection result of F0 generation mice by primers R-GT-F and R-GT-R. M is marker. H₂O is water control. WT is wildtype control. PC is positive control. F0-01, F0-02, F0-03, F0-04, F0-05, F0-06, F0-07, F0-08, F0-09, and F0-10 are mouse numbers.

FIG. 12A shows 5′ end PCR detection result of F1 generation mice by primers L-GT-F and L-GT-R. M is marker. H₂O is water control. WT is wildtype control. PC is positive control. F1-01, F1-02, F1-03, F1-04, F1-05, F1-06 and F1-07 are positive mouse numbers.

FIG. 12B shows 3′ end PCR detection result of F1 generation mice by primers R-GT-F and R-GT-R. M is marker. H₂O is water control. WT is wildtype control. PC is positive control. F1-01, F1-02, F1-03, F1-04, F1-05, F1-06, and F1-07 are positive mouse numbers.

FIG. 13 shows Southern Blot analysis result of F1 generation mice by P1 or P2 probe. M is marker. WT is wildtype control. F1-01, F1-02, F1-03, F1-04, F1-05, F1-06, and F1-07 are mouse numbers.

FIG. 14A shows a flow cytometry result of spleen cells from unstimulated wildtype C57BL/6 mouse. The spleen cells were stained with anti-mouse B2M antibody mβ2M PE and anti-mouse CD45 antibody mCD45 APC.

FIG. 14B shows a flow cytometry result of spleen cells from unstimulated MHC humanized homozygous mouse (H/H). The spleen cells were stained with anti-mouse B2M antibody mβ2M PE and anti-mouse CD45 antibody mCD45 APC.

FIG. 14C shows a flow cytometry result of spleen cells from wildtype C57BL/6 mouse stimulated by anti-mouse CD3 antibody. The spleen cells were stained with anti-mouse B2M antibody mβ2M PE and anti-mouse CD45 antibody mCD45 APC.

FIG. 14D shows a flow cytometry result of spleen cells from MHC humanized homozygous mouse (H/H) stimulated by anti-mouse CD3 antibody. The spleen cells were stained with anti-mouse B2M antibody mβ2M PE and anti-mouse CD45 antibody mCD45 APC.

FIG. 14E shows a flow cytometry result of spleen cells from unstimulated wildtype C57BL/6 mouse. The spleen cells were stained with anti-human B2M antibody hβ2M PE and anti-mouse CD45 antibody mCD45 APC.

FIG. 14F shows a flow cytometry result of spleen cells from unstimulated MHC humanized homozygous mouse (H/H). The spleen cells were stained with anti-human B2M antibody hβ2M PE and anti-mouse CD45 antibody mCD45 APC.

FIG. 14G shows a flow cytometry result of spleen cells from wildtype C57BL/6 mouse stimulated by anti-mouse CD3 antibody. The spleen cells were stained with anti-human B2M antibody hβ2M PE and anti-mouse CD45 antibody mCD45 APC.

FIG. 14H shows a flow cytometry result of spleen cells from MHC humanized homozygous mouse (H/H) stimulated by anti-mouse CD3 antibody. The spleen cells were stained with anti-human B2M antibody hβ2M PE and anti-mouse CD45 antibody mCD45 APC.

FIG. 14I shows a flow cytometry result of spleen cells from unstimulated wildtype C57BL/6 mouse. The spleen cells were stained with anti-mouse H-2Kb/H-2Db antibody and anti-mouse CD45 antibody mCD45 APC.

FIG. 14J shows a flow cytometry result of spleen cells from unstimulated MHC humanized homozygous mouse (H/H). The spleen cells were stained with anti-mouse H-2Kb/H-2Db antibody and anti-mouse CD45 antibody mCD45 APC.

FIG. 14K shows a flow cytometry result of spleen cells from wildtype C57BL/6 mouse stimulated by anti-mouse CD3 antibody. The spleen cells were stained with anti-mouse H-2Kb/H-2Db antibody and anti-mouse CD45 antibody mCD45 APC.

FIG. 14L shows a flow cytometry result of spleen cells from MHC humanized homozygous mouse (H/H) stimulated by anti-mouse CD3 antibody. The spleen cells were stained with anti-mouse H-2Kb/H-2Db antibody and anti-mouse CD45 antibody mCD45 APC.

FIG. 14M shows a flow cytometry result of spleen cells from unstimulated wildtype C57BL/6 mouse. The spleen cells were stained with anti-human HLA-A2 antibody hHLA-A2 PE and anti-mouse CD45 antibody mCD45 APC.

FIG. 14N shows a flow cytometry result of spleen cells from unstimulated MHC humanized homozygous mouse (H/H). The spleen cells were stained with anti-human HLA-A2 antibody hHLA-A2 PE and anti-mouse CD45 antibody mCD45 APC.

FIG. 14O shows a flow cytometry result of spleen cells from wildtype C57BL/6 mouse stimulated by anti-mouse CD3 antibody. The spleen cells were stained with anti-human HLA-A2 antibody hHLA-A2 PE and anti-mouse CD45 antibody mCD45 APC.

FIG. 14P shows a flow cytometry result of spleen cells from MHC humanized homozygous mouse (H/H) stimulated by anti-mouse CD3 antibody. The spleen cells were stained with anti-human HLA-A2 antibody hHLA-A2 PE and anti-mouse CD45 antibody mCD45 APC.

FIG. 15A shows a flow cytometry result of leukocytes in wildtype C57BL/6 mouse spleen cells. The spleen cells were stained with anti-mouse CD45 antibody mCD45 APC. The ratio of leukocytes was 88.6%.

FIG. 15B shows a flow cytometry result of leukocytes in MHC humanized homozygous mouse (H/H) spleen cells. The spleen cells were stained with anti-mouse CD45 antibody mCD45 APC. The ratio of leukocytes was 96.5%.

FIG. 15C shows a flow cytometry result of T cells and B cells in wildtype C57BL/6 mouse leukocytes. The T cells and B cells were stained with mouse T cell surface antibody mTCRB-APC-Cy7 and anti-mouse CD19 antibody mCD19-PE, respectively.

FIG. 15D shows a flow cytometry result of T cells and B cells in MHC humanized homozygous mouse (H/H) leukocytes. The T cells and B cells were stained with mouse T cell surface antibody mTCRB-APC-Cy7 and anti-mouse CD19 antibody mCD19-PE, respectively.

FIG. 15E shows a flow cytometry result of CD4+ T cells and CD8+ T cells in wildtype C57BL/6 mouse T cells. The CD4+ T cells and CD8+ T cells were stained with anti-mouse CD4 antibody mCD4-BV421 and the anti-mouse mCD8a antibody mCD8a-BV711, respectively.

FIG. 15F shows a flow cytometry result of CD4+ T cells and CD8+ T cells in MHC humanized homozygous mouse (H/H) T cells. The CD4+ T cells and CD8+ T cells were stained with anti-mouse CD4 antibody mCD4-BV421 and the anti-mouse mCD8a antibody mCD8a-BV711, respectively.

FIG. 16 shows PCR results from SIRPα knockout mice. M is Marker. WT is wildtype control. H₂O is water control. Numbers 1-10 are positive mouse numbers.

FIG. 17 is a schematic diagram showing humanized mouse B2M gene locus. Mouse B2M gene coding region was replaced with a nucleic acid sequence encoding the signal peptide of human HLA-A2.1, human B2M protein, and human HLA-A2.1 protein.

FIG. 18 shows a schematic diagram of a targeting strategy at mouse B2M gene locus.

FIG. 19A shows 5′ end PCR detection result of F0 generation mice by primers L-GT-F and BNDG-L-GT-R. M is marker. H₂O is water control. WT is wildtype control. PC is positive control. BNDG-F0-01, BNDG-F0-02, BNDG-F0-03, BNDG-F0-04, BNDG-F0-05, and BNDG-F0-06 are mouse numbers.

FIG. 19B shows 3′ end PCR detection result of F0 generation mice by primers BNDG-R-GT-F and R-GT-R. M is marker. H₂O is water control. WT is wildtype control. PC is positive control. BNDG-F0-01, BNDG-F0-02, BNDG-F0-03, BNDG-F0-04, BNDG-F0-05, and BNDG-F0-06 are mouse numbers.

FIG. 20A shows 5′ end PCR detection result of F1 generation mice by primers L-GT-F and BNDG-L-GT-R. M is marker. H₂O is water control. WT is wildtype control. PC is positive control. BNDG-F0-01 and BNDG-F0-02 are mouse numbers.

FIG. 20B shows 3′ end PCR detection result of F1 generation mice by primers BNDG-R-GT-F and R-GT-R. M is marker. H₂O is water control. WT is wildtype control. PC is positive control. BNDG-F0-01 and BNDG-F0-02 are mouse numbers.

FIG. 21 shows Southern Blot analysis result of F1 generation mice by BNDG-P1 or BNDG-P2 probe. M is marker. WT is wildtype control. BNDG-F0-01 and BNDG-F0-02 are mouse numbers.

FIG. 22A shows a flow cytometry result of spleen cells from B-NDG mouse. The spleen cells were stained with anti-mouse B2M antibody mβ2M PE and anti-mouse CD45 antibody mCD45 APC.

FIG. 22B shows a flow cytometry result of spleen cells from B-NDG background MHC humanized heterozygous mouse (H/+). The spleen cells were stained with anti-mouse B2M antibody mβ2M PE and anti-mouse CD45 antibody mCD45 APC.

FIG. 22C shows a flow cytometry result of spleen cells from B-NDG mouse. The spleen cells were stained with anti-human B2M antibody hβ2M PE and anti-mouse CD45 antibody mCD45 APC.

FIG. 22D shows a flow cytometry result of spleen cells from B-NDG background MHC humanized heterozygous mouse (H/+). The spleen cells were stained with anti-human B2M antibody hβ2M PE and anti-mouse CD45 antibody mCD45 APC.

FIG. 22E shows a flow cytometry result of spleen cells from B-NDG mouse. The spleen cells were stained with anti-mouse H-2Kb/H-2Db antibody and anti-mouse CD45 antibody mCD45 APC.

FIG. 22F shows a flow cytometry result of spleen cells from B-NDG background MHC humanized heterozygous mouse (H/+). The spleen cells were stained with anti-mouse H-2Kb/H-2Db antibody and anti-mouse CD45 antibody mCD45 APC.

FIG. 22G shows a flow cytometry result of spleen cells from B-NDG mouse. The spleen cells were stained with anti-human HLA-A2 antibody hHLA-A2 PE and anti-mouse CD45 antibody mCD45 APC.

FIG. 22H shows a flow cytometry result of spleen cells from B-NDG background MHC humanized heterozygous mouse (H/+). The spleen cells were stained with anti-human HLA-A2 antibody hHLA-A2 PE and anti-mouse CD45 antibody mCD45 APC.

FIG. 23 shows the alignment between mouse B2M amino acid sequence (NP_033865.2; SEQ ID NO: 2) and human B2M amino acid sequence (NP_004039.1; SEQ ID NO: 4).

FIG. 24 shows the alignment between rat B2M amino acid sequence (NP_036644.1; SEQ ID NO: 66) and human B2M amino acid sequence (NP_004039.1; SEQ ID NO: 4).

FIG. 25 shows the alignment between mouse H2-D1 amino acid sequence (NP_034510.3; SEQ ID NO: 6) and human HLA-A2.1 amino acid sequence (AAC24825.1; SEQ ID NO: 59).

FIG. 26 shows the alignment between mouse H2-D1 amino acid sequence (NP_034510.3; SEQ ID NO: 6) and human HLA-A*0101 amino acid sequence (NP_001229687.1; SEQ ID NO: 8).

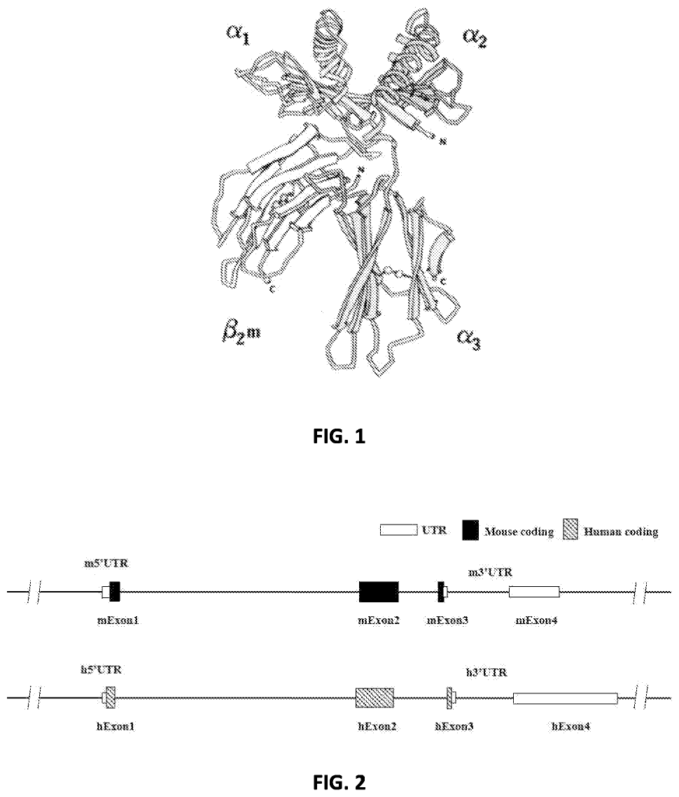

### EXAMPLES

The invention is further described in the following examples, which do not limit the scope of the invention described in the claims.

### Materials and Methods

The following materials were used in the following examples.

NOD-Prkdc^{scid} IL-2rg^{null} (B-NDG) mice were obtained from Beijing Biocytogen Co., Ltd. The catalog number is B-CM-001 or B-CM-002.

UCA kit was obtained from Beijing Biocytogen Co., Ltd. The catalog number is BCG-DX-001.

Ambion™ in vitro transcription kit was purchased from Ambion, Inc. The catalog number is AM1354.

Cas9 mRNA was obtained from SIGMA. The catalog number is CAS9MRNA-1EA.

PE anti-human β2-microglobulin Antibody (hβ2M PE) was purchased from BioLegend. The catalog number is 316305.

PE anti-mouse β2-microglobulin Antibody (mβ2M PE) was purchased from BioLegend. The catalog number is 154503.

PE anti-human HLA-A2 Antibody (hHLA-A2 PE) was purchased from BioLegend. The catalog number is 343305.

PE anti-mouse H-2K^{b}/H-2D^{b} Antibody (H-2Kb/H-2Db PE) was purchased from BioLegend. The catalog number is 114607.

FITC anti-mouse CD19 Antibody (mCD19FITC) was purchased from BioLegend. The catalog number is 115506.

Purified anti-mouse CD16/32 Antibody was purchased from BioLegend. The catalog number is 101302.

APC anti-mCD45 (mCD45APC) was purchased from BioLegend. The catalog number is 559864.

APC/Cy7 anti-mouse TCR R chain Antibody (mTCRβ APC/Cy7) was purchased from BioLegend. The catalog number is 109220.

Alexa Fluor® 488 anti-mouse CD3 Antibody (mCD3 Alexa Flour 488) was purchased from Biolegend. The catalog number is 100210.

PE anti-mouse CD19 Antibody (mCD19 PE) was purchased from Biolegend. The catalog number is 115508.

Brilliant Violet 421™ anti-mouse CD4 Antibody (mCD4 BV421) was purchased from BioLegend. The catalog number is 100438.

Brilliant Violet 711™ anti-mouse CD8a Antibody (mCD8a BV711) was purchased from BioLegend. The catalog number is 100747.

BamHI, BglII, EcoNI, and SspI restriction enzymes were purchased from NEB. The catalog numbers are R3136, R0144, R0521, and R3132, respectively.

### Example 1: Generation of MHC Humanized Mice

The genome of a non-human animal (e.g., a mouse) can be modified to include a nucleic acid sequence encoding all or a part of a human B2M and HLA-A2.1 proteins, such that the genetically modified non-human animal can express human or humanized B2M and HLA-A2.1 proteins. The mouse B2M gene (NCBI Gene ID: 12010, Primary source: MGI: 88127, UniProt ID: P01887) is located in chromosome 2 of the mouse genome (from 122,147,686 to 122,153,083 of NC_000068.7). The transcript sequence NM_009735.3 is set forth in SEQ ID NO: 1, and the corresponding protein sequence NP_033865.2 is set forth in SEQ ID NO: 2. The human B2M gene (NCBI Gene ID: 567, Primary source: HGNC: 914, UniProt ID: P61769) is located in chromosome 15 of the human genome (from 44,711,487 to 44,718,877 of NC_000015.10). The transcript sequence NM_004048.3 is set forth in SEQ ID NO: 3, and the corresponding protein sequence NP_004039.1 is set forth in SEQ ID NO: 4. Mouse and human B2M gene loci are shown in FIG. 2.

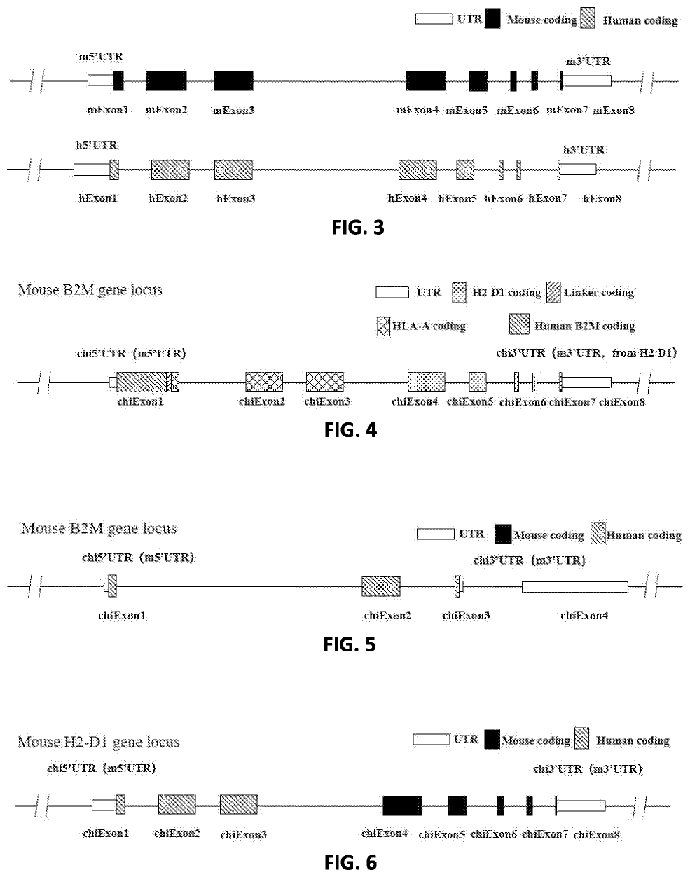

The mouse H2-D1 gene (NCBI Gene ID: 14964, Primary source: MGI: 95896, UniProt ID: P01899) is located in chromosome 17 of the mouse genome. The transcript sequence NM_010380.3 is set forth in SEQ ID NO: 5, and the corresponding protein sequence NP_034510.3 is set forth in SEQ ID NO: 6. The human HLA-A gene (NCBI Gene ID: 3105, Primary source: HGNC: 4931, UniProt ID: P04439) is located in chromosome 6 of the human genome. The transcript sequence NM_001242758.1 is set forth in SEQ ID NO: 7, and the corresponding protein sequence NP_001229687.1 is set forth in SEQ ID NO: 8. Mouse H2-D1 gene locus and human HLA-A gene locus are shown in FIG. 3.

To obtain a transgenic mouse expressing a human or humanized MHC molecule, various strategies can be used. For example, the mouse endogenous B2M gene and endogenous H2-D1 gene can be inactivated, and a nucleic acid sequence encoding a polypeptide sequence including: human B2M; the signal peptide and a portion of the extracellular region of human HLA-A2.1 (e.g., amino acids 1-203 of SEQ ID NO: 59); a portion of the extracellular region (Alpha-3 region and connecting peptide), the transmembrane region and the cytoplasmic region of mouse H2-D1 (e.g., amino acids 207-362 of SEQ ID NO: 6) can be knocked into mouse genome.

Different modifications and combinations can also be used on the mouse B2M and/or mouse H2-D1 gene loci.

For example, the mouse endogenous B2M gene locus can be humanized as follows. As shown in FIG. 4, within exon 1 of the mouse endogenous B2M gene, a nucleic acid sequence encoding a polypeptide including: human B2M; the signal peptide, Alpha-1, and Alpha-2 regions of human HLA-A2.1 (e.g., amino acids 1-203 of AAC24825.1 (SEQ ID NO: 59) encoded by human HLA-A2.1 exons 1-3); the Alpha-3 region, connecting peptide, transmembrane region, and the cytoplasmic region of mouse H2-D1 (e.g., amino acids 207-362 of NP_034510.3 (SEQ ID NO: 6) encoded by mouse H2-D1 exons 4-8) can be used to replace a sequence spanning exons 1-3 the mouse endogenous B2M gene.

As a different strategy, the mouse endogenous B2M gene can be directly humanized. As shown in FIG. 5, the coding region of mouse B2M gene can be replaced with the coding region of human B2M gene. Meanwhile, a sequence encoding a polypeptide (SEQ ID NO: 64) including: the signal peptide, Alpha-1, and Alpha-2 regions of human HLA-A2.1; the Alpha-3 region, connecting peptide, transmembrane region, and the cytoplasmic region of mouse H2-D1 can be knocked into mouse genome by transgenic techniques. Alternatively, as shown in FIG. 6, at mouse endogenous H2-D1 gene locus, a sequence encoding a polypeptide including the signal peptide, Alpha-1, and Alpha-2 regions of human HLA-A2.1 can be used to replace a corresponding sequence of mouse H2-D1 gene encoding the signal peptide, Alpha-1 and Alpha-2 regions. When the mouse B2M gene and H2-D1 gene are respectively humanized, double-gene humanized mice can be prepared by one-step or multistep targeting strategies. It is also possible to prepare single-gene humanized mice separately, and obtain double-gene humanized mice through methods such as breeding. The obtained double-gene humanized mice can simultaneously express human B2M protein and humanized MHC α chain protein in vivo.

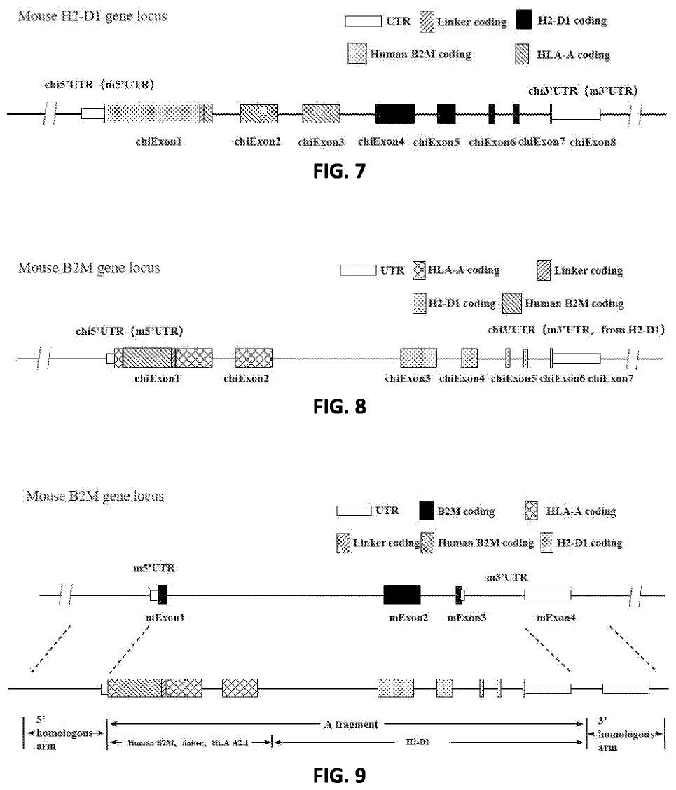

As a different strategy, the mouse endogenous B2M gene can be knocked out. Meanwhile, as shown in FIG. 7, at mouse endogenous H2-D1 gene locus, a sequence encoding a polypeptide (SEQ ID NO: 63) including human B2M; the signal peptide, Alpha-1, and Alpha-2 regions of human HLA-A2.1 can be used to replace a sequence encoding the signal peptide, Alpha-1 and Alpha-2 regions of mouse H2-D1.

In the following experiment, the humanization method shown in FIG. 4 was used to generate transgenic mice with humanized MHC molecules. Gene editing technology can be used to modify mouse cells. The endogenous mouse B2M gene locus can be knocked into a sequence encoding human B2M protein, a portion of human HLA-A2.1 protein, and a portion of mouse H2-D1 protein, which can also disrupt the mouse B2M gene coding region. The generated humanized mice can express humanized MHC molecules in vivo, which contains: human B2M protein; the Alpha-1 and Alpha-2 regions of human HLA-A2.1 protein (NCBI reference sequence: AAC24825.1, SEQ ID NO: 59); the Alpha-3 region, connecting peptide, the transmembrane region, and the cytoplasmic region of mouse H2-D1 protein. The human HLA-A2.1 protein portion is directly connected to the mouse H2-D1 protein portion, and the humanized mice does not express endogenous B2M protein. Further, in order to ensure correct expression of human HLA-A2.1 protein, a sequence encoding the signal peptide of human HLA-A2.1 protein can also be inserted before the human B2M coding region. The schematic diagram of the humanized mouse B2M locus is shown in FIG. 8. The mRNA sequence transcribed from the humanized B2M, HLA-A2.1 and H2-D1 genes is shown in SEQ ID NO: 9, and the DNA sequence of the humanized B2M locus (only the modified part) is shown in SEQ ID NO: 10.

Given that human B2M have multiple isoforms or transcripts, the methods described herein can be applied to other isoforms or transcripts.

The CRISPR/Cas system was applied for gene editing, and the targeting strategy is shown in FIG. 9. A targeting vector was designed, containing homologous arm sequences upstream and downstream of mouse B2M gene, and an “A fragment” encoding human B2M protein, a portion of human HLA-A2.1 protein, and a portion of mouse H2-D1 protein. The upstream homologous arm sequence (5′ homologous arm, SEQ ID NO: 11) is identical to nucleic acids 122146329-122147737 of NCBI reference sequence NC_000068.7. The downstream homologous arm sequence (3′ homologous arm, SEQ ID NO: 12) is identical to nucleic acids 122152171-122153513 of NCBI reference sequence NC_000068.7. The “A fragment” contains sequences from 5′ end to 3′ end that encode the following polypeptides: the signal peptide of human HLA-A2.1; human B2M; a flexible linker polypeptide sequence; a portion of the human HLA-A2.1 protein; and a portion of the mouse H2-D1 protein. Specifically, the sequence encoding the signal peptide of human HLA-A2.1 (SEQ ID NO: 13) is identical to nucleic acids 99567-99638 of GenBank reference sequence AF055066.1. The sequence encoding human B2M (SEQ ID NO: 14) is identical to nucleic acids 91-387 of NCBI reference sequence NM_004048.3. The flexible linker polypeptide sequence is the (GGGGS)₃ (SEQ ID NO: 67) linker that is encoded by a 45 bp sequence 5′-GGAGGTGGCGGATCCGGCGGAGGCGGCTCGGGTGGCGGCGGCTCT-3′ (SEQ ID NO: 51). The portion of human HLA-A2.1 protein is encoded by a sequence (SEQ ID NO: 15) that is identical to nucleic acids 98606-99435 of GenBank reference sequence AF055066.1. The portion of mouse H2-D1 protein is encoded by a sequence (SEQ ID NO: 16) that is identical to nucleic acids 35266871-35267765 of NCBI reference sequence NC_000083.6. The protein expressed in the transgenic mice is shown in SEQ ID NO: 61. However, due to the existence of the flexible linker polypeptide sequence, the protein can have the functional domains of human B2M and HLA-A2.1 protein.

The targeting vector was constructed, e.g., by restriction enzyme digestion/ligation, or gene synthesis. The constructed targeting vector sequence was preliminarily verified by restriction enzyme digestion, then verified by sequencing. The verified targeting vector was used for subsequent experiments.

The target sequences are important for the targeting specificity of sgRNAs and the efficiency of Cas9-induced cleavage. Specific sgRNA sequences were designed and synthesized that recognize the 5′ end targeting site (sgRNA1-sgRNA7) and 3′ end targeting site (sgRNA8-sgRNA15). The 5′ end targeting site is located within the first exon or the first intron of the mouse B2M gene. The 3′ end targeting site is located on the third exon or third intron of the mouse B2M gene. The targeting site sequence of each sgRNA on the B2M gene locus is as follows:

sgRNA1 targeting site (SEQ ID NO: 17):
5′-CCTGGCCAATCCCGTCGGGAAGG-3′
sgRNA2 targeting site (SEQ ID NO: 18):
5′-CCGTCAGCACACTCGCAAACAGG-3′
sgRNA3 targeting site (SEQ ID NO: 19):
5′-GTTCTCCTTCCCGACGGGATTGG-3′
sgRNA4 targeting site (SEQ ID NO: 20):
5′-ACTCTGGATAGCATACAGGCCGG-3′
sgRNA5 targeting site (SEQ ID NO: 21):
5′-CTGGTGCTTGTCTCACTGACCGG-3′
sgRNA6 targeting site (SEQ ID NO: 22):
5′-GGGGAAAGAGGCACTCACTCTGG-3′
sgRNA7 targeting site (SEQ ID NO: 23):
5′-GACAAGCACCAGAAAGACCAGGG-3′
sgRNA8 targeting site (SEQ ID NO: 24):
5′-CTGGAGGCTTCCGGACACTCAGG-3′
sgRNA9 targeting site (SEQ ID NO: 25):
5′-TGATCAAGCATCATGATGGTAGG-3′
sgRNA10 targeting site (SEQ ID NO: 26):
5′-AGGAGCGTGAGAGGGAACGTGGG-3′
sgRNA11 targeting site (SEQ ID NO: 27):
5′-GAGGAACGTAGCCATGTCACTGG-3′
sgRNA12 targeting site (SEQ ID NO: 28):
5′-CATGTCACTGGCCCTCTAAAGGG-3′
sgRNA13 targeting site (SEQ ID NO: 29):
5′-CATGTGATCAAGCATCATGATGG-3′
sgRNA14 targeting site (SEQ ID NO: 30):
5′-ACCCGCAGAGCTCTGTCACTCGG-3′
sgRNA15 targeting site (SEQ ID NO: 31):
5′-CTCTGTCACTCGGCTCCTCTGGG-3′

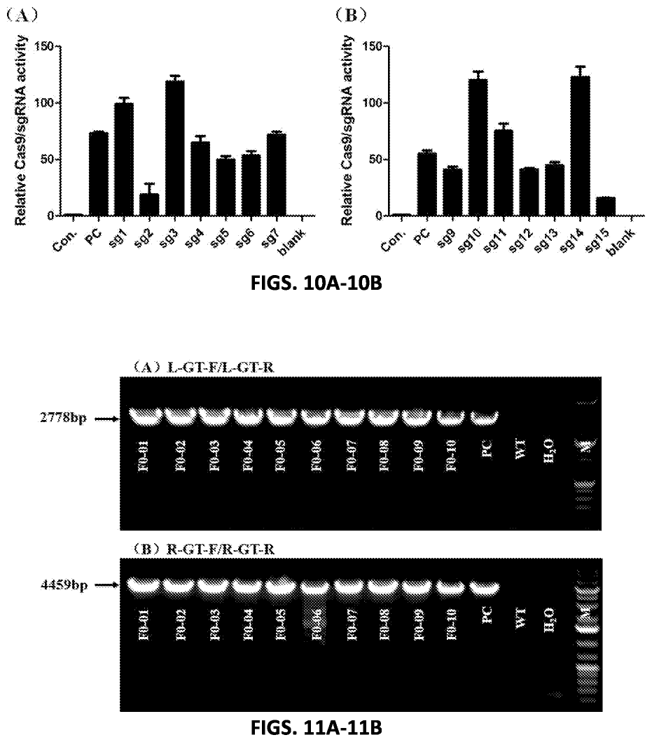

The UCA kit was used to detect the activities of sgRNAs. The results showed that the sgRNAs had different activities. The results are shown in Table 5 and FIGS. 10A-10B. sgRNA3 and sgRNA14 were selected for subsequent experiments. Oligonucleotides were added to the 5′ end and a complementary strand to obtain a forward oligonucleotide and a reverse oligonucleotide (see Table 6 for the sequences). After annealing, the products were ligated to the pT7-sgRNA plasmid (the plasmid was first linearized with BbsI), respectively, to obtain expression vectors PT7-B2M-HLA-A2.1-3 and pT7-B2M-HLA-A2.1-14.

TABLE 5
UCA test results showing sgRNA activity

| | |
|---|---|
| 5′ end targeting site | 3′ end targeting site |
| detection result | detection result |

| | | | |
|---|---|---|---|
| Con. | 1.00 ± 0.06 | Con. | 1.00 ± 0.03 |
| PC | 73.04 ± 1.51 | PC | 55.01 ± 2.95 |
| sgRNA1 | 99.08 ± 5.37 | sgRNA8 | 143.48 ± 8.70 |
| sgRNA2 | 18.89 ± 9.70 | sgRNA9 | 41.19 ± 2.51 |
| sgRNA3 | 118.85 ± 5.15 | sgRNA10 | 120.32 ± 7.44 |
| sgRNA4 | 64.87 ± 5.75 | sgRNA11 | 75.28 ± 6.24 |
| sgRNA5 | 49.91 ± 3.03 | sgRNA12 | 41.31 ± 1.12 |
| sgRNA6 | 53.58 ± 3.78 | sgRNA13 | 44.85 ± 2.75 |
| sgRNA7 | 71.85 ± 2.74 | sgRNA14 | 122.80 ± 9.26 |
| — | − | sgRNA15 | 15.83 ± 0.39 |

TABLE 6
sgRNA3 and sgRNA14 sequence list
sgRNA3 sequences

| | |
|---|---|
| SEQ ID NO: 32 | Upstream: 5′-GTTCTCCTTCCCGACGGGAT-3′ |
| SEQ ID NO: 33 | Upstream: 5′-TAGGTTCTCCTTCCCGACGGGAT-3′ |
| (forward oligonucleotide) | |
| SEQ ID NO: 34 | Downstream: 5′-ATCCCGTCGGGAAGGAGAA-3′ |
| SEQ ID NO: 35 | Downstream: 5′-AAACATCCCGTCGGGAAGGAGAA-3′ |
| (reverse oligonucleotide) | |

sgRNA14 sequences

| | |
|---|---|
| SEQ ID NO: 36 | Upstream: 5′-ACCCGCAGAGCTCTGTCACT-3′ |
| SEQ ID NO: 37 | Upstream: 5′-TAGGACCCGCAGAGCTCTGTCACT-3′ |
| (forward oligonucleotide) | |
| SEQ ID NO: 38 | Downstream: 5′-AGTGACAGACTCTGCGGGT-3′ |
| SEQ ID NO: 39 | Downstream: 5-AAACAGTGACAGACTCTGCGGGT-3′ |
| (reverse oligonucleotide) | |

T7 promoter and sgRNA scaffold (SEQ TD NO: 40), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, in vitro transcription products of the pT7-B2M-HLA-A2.1-3 and pT7-B2M-HLA-A2.1-14 plasmids (using Ambion in vitro transcription kit to carry out the transcription according to the method provided in the product instruction) were injected into the cytoplasm or nucleus of C57BL/6 mouse fertilized eggs with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable mouse lines with human or humanized B2M and HLA-A2.1 gene.

Experiments were performed to identify somatic cell genotype of the F0 generation mice. For example, PCR analysis was performed using mouse tail genomic DNA of the F0 generation mice. The PCR analysis results for some of the F0 mice are shown in FIGS. 11A-11B. In view of the 5′ end primer detection result and the 3′ end primer detection result, the 10 mice numbered from F0-01 to F0-10 were all positive mice.

The following primers were used in the PCR:

5′ end primers:
L-GT-F (SEQ ID NO: 41):
5′-GAATGTGTGCCTCCTCTCAGTTTCC-3′
L-GT-R (SEQ ID NO: 42):
5′-TCCTTCCCGTTCTCCAGGTATCTGC-3′
3′ end primers:
R-GT-F (SEQ ID NO: 43):
5′-GCGGCTACTACAACCAGAGCGAG-3′
R-GT-R (SEQ ID NO: 44):
5′-TCCAGCAATAAGAACCAGTCCCTAGCT-3′

The primer L-GT-F is located on the left side of the 5′ homologous arm. R-GT-R is located on the right side of the 3′ homologous arm. Both L-GT-R and R-GT-F are located on the human sequence.

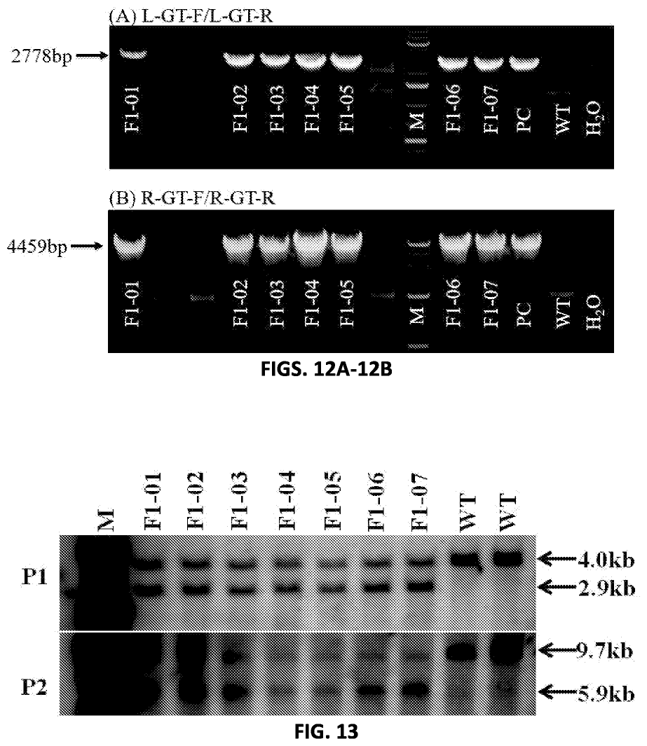

The positive F0 generation MHC humanized mice were bred with wildtype mice to generate F1 generation mice. The same method (e.g., PCR) was used for genotypic identification of the F1 generation mice. As shown in FIGS. 12A-12B, 7 mice numbered F1-01, F1-02, F1-03, F1-04, F1-05, F1-06, and F1-07 were identified as positive mice. The 7 positive F1 generation mice were further analyzed by Southern Blot, to confirm if random insertions were introduced. Specifically, mouse tail genomic DNA was extracted, digested with BamHI or BglII restriction enzyme, transferred to a membrane, and then hybridized with probes. Probes P1 and P2 are located on the upstream region of the 5′ homologous arm and on the 3′ homologous arm, respectively. The probes used in Southern Blot assays are listed in the table below.

TABLE 7

| Restriction enzyme | Probe | WT size | Targeted size |
|---|---|---|---|
| BamHI | P1 | 9.7 kb | 5.9 kb |
| BglII | P2 | 4.0 kb | 2.9 kb |

The probes were synthesized using the following primers:

P1-F (SEQ ID NO: 45):
5′-ATGAGGTCTTTTTGTGGGCAGAGCA-3′
P1-R (SEQ ID NO: 46):
5′-CTCCCTACGGCCACATCACCATTAC-3′
P2-F (SEQ ID NO: 47):
5′-TAACTTCATGTAAGGCACCGTCAC-3′
P2-R (SEQ ID NO: 48):
5′-TCCAGACCTCACCATCAAATGAG-3′

The detection result of Southern Blot is shown in FIG. 13. In view of the hybridization results by P1 and P2 probes, the seven F1 generation mice were confirmed to be positive heterozygotes and no random insertions were detected. This indicates that the method described above can be used to generate genetically-modified MHC gene humanized mice that can be stably passaged without random insertions.

The heterozygous mice identified as positive in the F1 generation can be bred with each other to obtain the F2 generation MHC humanized homozygous mouse (H/H).

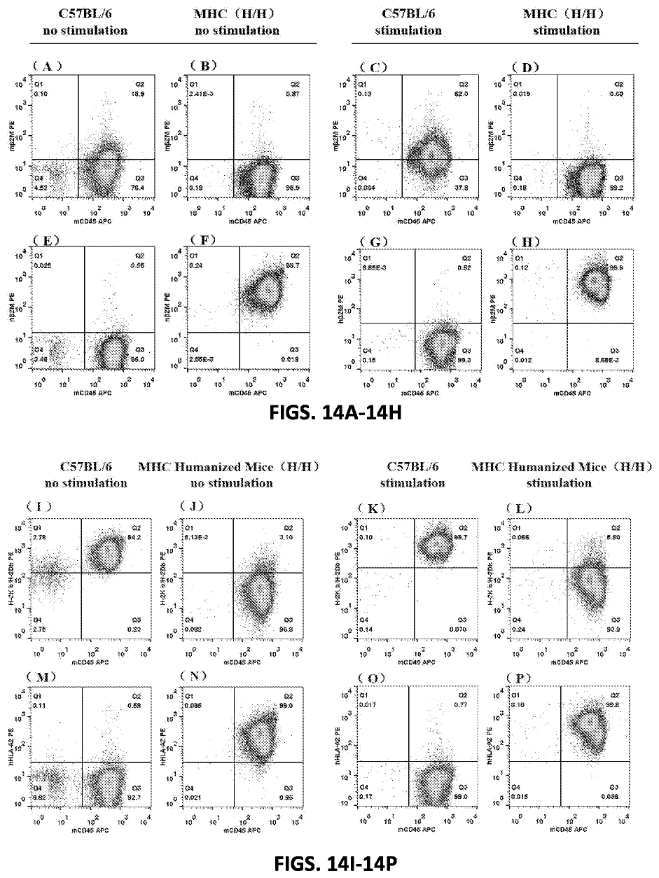

The expression of human B2M protein and HLA-A2.1 protein in positive mice was confirmed by ELISA. Specifically, one wildtype C57BL/6 female mouse (6-week old) and one MHC humanized female homozygous mouse (6-week old) prepared by the method described herein were selected, and each mouse was injected intraperitoneally with 7.5 μg (volume: 200 l) anti-mouse CD3 antibody. After 24 hours, the mice were sacrificed and then spleen cells were collected. Anti-mouse B2M antibody mβ2M PE, anti-mouse H-2Kb/H-2Db antibody, anti-human B2M antibody hβ2M PE, or anti-human HLA-A2 antibody hHLA-A2 PE; together with anti-mouse CD45 antibody mCD45 APC were used for spleen cell staining. The stained cells were subjected to flow cytometry analysis with results shown in FIGS. 14A-14P. Regardless of whether the cells were stimulated by anti-mouse CD3 antibody, only mouse B2M-expressing cells were detected in C57BL/6 mice (FIGS. 14A and 14C), human or humanized B2M-expressing cells were not detected in C57BL/6 mice (FIGS. 14E and 14G). Cells expressing human B2M (FIGS. 14F and 14H), and cells expressing HLA-A2.1 (FIGS. 14N and 14P) were only detected in MHC humanized homozygous mice. However, cells expressing mouse B2M were not detected in MHC humanized homozygous mice (FIGS. 14B and 14D). In addition, because the mouse H2-D1 coding sequence was not knocked out, a small number of cells expressing mouse H2-D1 were detected in MHC humanized homozygous mice (FIGS. 14G and 14L).

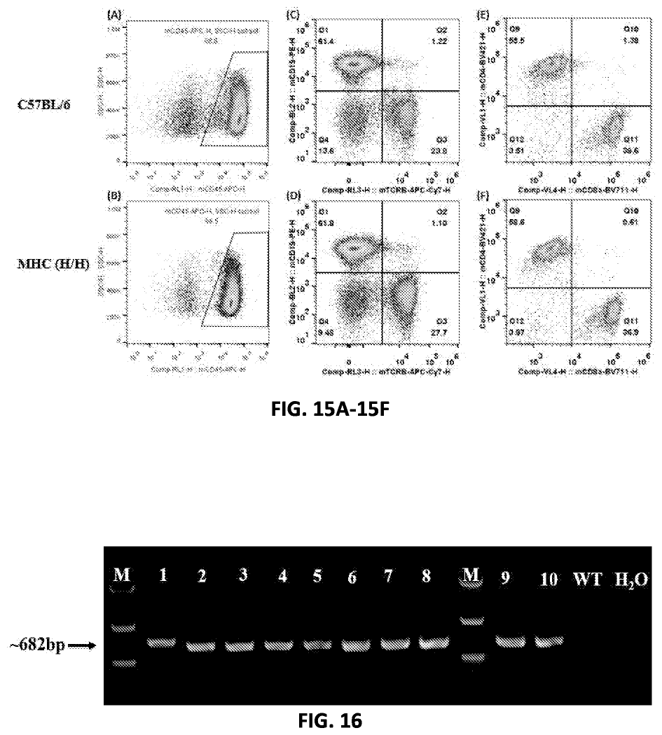

To confirm whether the differentiation of B cells and T cells in F2 generation MHC humanized homozygous mice was consistent with that of wildtype mice, the mouse lymphocyte subsets were analyzed by flow cytometry. Specifically, one wildtype C57BL/6 male mouse (16-week old) and one MHC humanized homozygous male mouse (13-week old) were selected respectively. The spleen cells were collected, and anti-mouse CD45 antibody mCD45 APC was used for cell staining and flow cytometry detection. As shown in FIGS. 15A-15B, the ratios of leukocytes in wildtype C57BL/6 mice and MHC humanized homozygous mice were 88.6% and 96.5%, respectively. Subsequently, mouse T cell surface antibody mTCRB-APC-Cy7 and anti-mouse CD19 antibody mCD19-PE were used to stain T cells and B cells, respectively, for flow cytometry analysis. The results showed that the T cells and B cells in the wildtype C57BL/6 mice were 23.8% and 61.4%, respectively (FIG. 15C); whereas the T cells and B cells in the humanized MHC homozygous mice were 27.7% and 61.8%, respectively (FIG. 15D). Further, anti-mouse CD4 antibody mCD4-BV421 and the anti-mouse mCD8a antibody mCD8a-BV711 were used for cell staining, and the stained cells were subjected to flow cytometry analysis. As shown in FIGS. 15E-15F, the ratios of CD4+ T cells and CD8+ T cells in wildtype C57BL/6 mice and MHC humanized homozygous mice were comparable.

In summary, the above results showed that the expression of lymphocyte subsets in MHC humanized mice was similar to that of wildtype C57BL/6 mice. The results further indicated that the differentiation of T cells and B cells in MHC humanized mice was not affected by humanization of B2M and HLA-A2.1 genes.

Since the cleavage of Cas9 results in DNA double strand break, and the homologous recombination repair may result in insertion/deletion mutations, it is possible to obtain B2M gene knockout mice using the method described herein. A pair of primers was designed to detect the gene knockout mice. Wildtype mice should have no PCR bands, and knockout mice should have one PCR band at about 682 bp. As shown in FIG. 16, mice numbered 1-10 were identified as B2M gene knockout mice. One PCR primer was located on the left side of the 5′ targeting site, and the other PCR primer was located on the right side of the 3′ targeting site. The primers are shown below:

SEQ ID NO: 49:
5′-GAATAAATGAAGGCGGTCCCAGGCT-3′
SEQ ID NO: 50:
5′-AGGTGAGTTCTGGCTCCACCATTTG-3′

### Example 2. MHC Humanized Mice with Severe Immunodeficiency

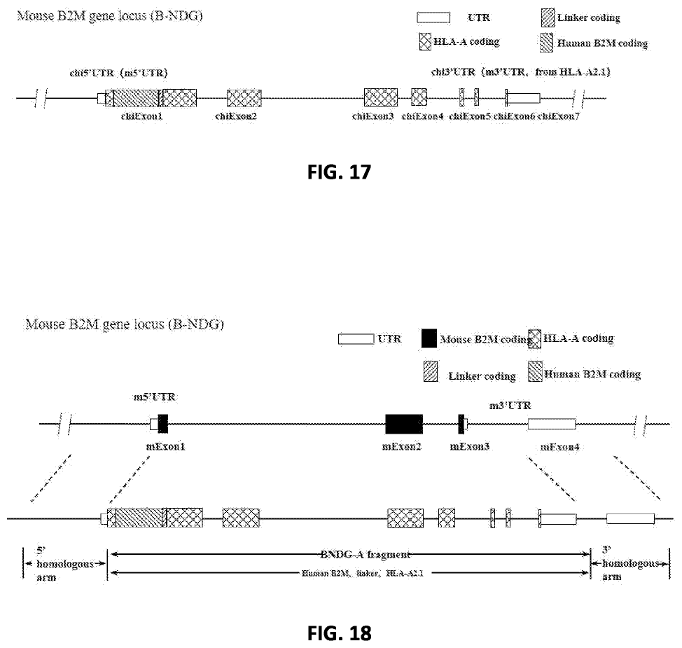

In addition to the humanization strategy as described in Example 1, mice with a higher degree of humanization of MHC molecules can also be designed. Furthermore, immunodeficient mice with humanized MHC molecules can be designed to provide effective experimental animal models for the research of pathogenesis mechanisms of immune system diseases, such as diabetes and transplant rejection, and drug development. In this example, B-NDG background mice were used to carry out a higher degree of humanization of MHC molecules. Specifically, gene editing technology was used to modify B-NDG mice. The endogenous mouse B2M gene locus was knocked into a sequence encoding human B2M protein and HLA-A2.1 protein, which also disrupted the mouse B2M gene coding region. The generated humanized mice can express humanized MHC molecules in vivo, containing human B2M protein and human HLA-A2.1 protein. The humanized mice did not express endogenous B2M protein. Further, in order to ensure correct expression of human HLA-A2.1 protein, a sequence encoding the signal peptide of human HLA-A2.1 protein was inserted before the human B2M coding region. The schematic diagram of the humanized mouse B2M locus is shown in FIG. 17. The mRNA sequence transcribed from the humanized B2M and HLA-A2.1 genes is shown in SEQ ID NO:52. The protein expressed by the transgenic mice is shown in SEQ ID NO: 62. However, due to the existence of the flexible linker polypeptide sequence, the protein can have the functional domains of human B2M protein and human HLA-A2.1 protein.

Given that human B2M gene have multiple isoforms or transcripts, the methods described herein can be applied to other isoforms or transcripts.

Further, a schematic diagram of the targeting strategy is shown in FIG. 18. The targeting vector is similar to the targeting vector used in Example 1, except that: the 5′ homologous arm (SEQ ID NO: 60) has 99% homology with nucleic acids 122146329-122147737 of NCBI reference number NC_000068.7, with mutations at position 122147015 (from G to T), position 122147108 (from C to T), and position 122147591 (from C to T); the 3′ homologous arm (SEQ ID NO: 53) has 99% homology with nucleic acids 122152171-122153513 of NCBI reference number NC_000068.7, with mutations at position 122152258 (from G to A), position 122152391 (from G to A), position 122152771 (from A to G), position 122153104 (from T to C), and position 122153148 (from A to C); and deletion at position 122152788 (deletion of A). the BNDG-A fragment (SEQ ID NO: 65) is similar to the “A fragment” in Example 1, but contains a sequence encoding the full-length human HLA-A2.1 protein. The sequence (SEQ ID NO: 54) encoding the full-length human HLA-A2.1 protein is identical to nucleic acids 95493-99436 of GenBank reference sequence AF055066.1. The BNDG-A fragment does not contain any sequence encoding moues H2-D1 protein.

The targeting vector was constructed, e.g., by restriction enzyme digestion/ligation, or gene synthesis. The constructed targeting vector sequence was preliminarily verified by restriction enzyme digestion, then verified by sequencing. The verified targeting vector was used for subsequent experiments (e.g., microinjection). The sgRNAs used was the same as the sgRNAs used in Example 1.

Specifically, the pre-mixed Cas9 mRNA, the targeting vector, in vitro transcription products of the pT7-B2M-HLA-A2.1-3 and pT7-B2M-HLA-A2.1-14 plasmids (using Ambion in vitro transcription kit to carry out the transcription according to the method provided in the product instruction) were injected into the cytoplasm or nucleus of B-NDG mouse fertilized eggs with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable B-NDG background mouse lines with human B2M and HLA-A2.1 genes.

Experiments were performed to identify somatic cell genotype of the F0 generation mice with B-NDG background. For example, PCR analysis was performed using mouse tail genomic DNA of the F0 generation mice. The PCR analysis results for some of the F0 mice are shown in FIGS. 19A-19B. In view of the 5′ end primer detection result and the 3′ end primer detection result, the 6 mice numbered from BNDG-F0-01 to BNDG-F0-06 were all positive mice.

The following primers were used in the PCR:

5′ end primers:
L-GT-F (SEQ ID NO: 41):
5′-GAATGTGTGCCTCCTCTCAGTTTCC-3′
BNDG-L-GT-R (SEQ ID NO: 55):
5′-CAGCTCCAAAGAGAACCAGGCCAG-3′
3′ end primers:
BNDG-R-GT-F (SEQ ID NO: 56):
5′-TACCCTGCGGAGATCACACTGACC-3′
R-GT-R (SEQ ID NO: 44):
5′-TCCAGCAATAAGAACCAGTCCCTAGCT-3′

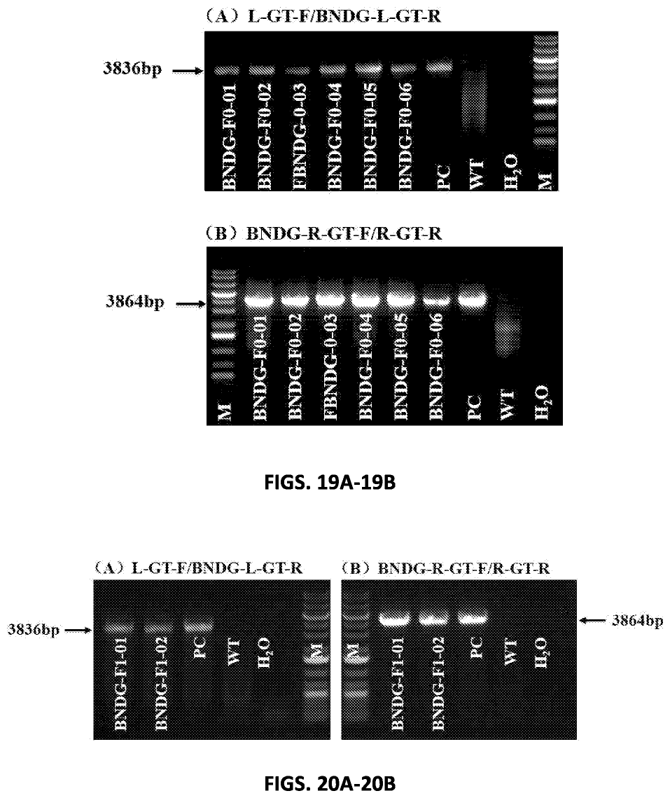

The positive F0 generation MHC humanized mice were bred with wildtype mice to generate F1 generation mice. The same method (e.g., PCR) was used for genotypic identification of the F1 generation mice. As shown in FIGS. 20A-20B, 2 mice numbered BNDG-F1-01 and BNDG-F1-02 were identified as positive mice. The 2 positive F1 generation mice were further analyzed by Southern Blot, to confirm if random insertions were introduced. Specifically, mouse tail genomic DNA was extracted, digested with EcoNI or SspI restriction enzyme, transferred to a membrane, and then hybridized with probes. Probes BNDG-P1 and BNDG-P2 are located on the upstream region of the 5′ homologous arm and on the 3′ homologous arm, respectively. The probes used in Southern Blot assays are listed in the table below.

TABLE 8

| Restriction enzyme | Probe | WT size | | Targeted size |
|---|---|---|---|---|
| EcoNI | BNDG-P1 | 8.8 | kb | 5.3 kb |
| SspI | BNDG-P2 | 12.1 | kb | 7.6 kb |

The probes were synthesized using the following primers:

BNDG-P1-F (SEQ ID NO: 57):
5′-TTCTGATGCTCCTTCCTTCCGTGC-3′
BNDG-P1-R (SEQ ID NO: 58):
5′-TTCTCTGTGCTCAGTGTTCCCTGC-3′
BNDG-P2-F (SEQ ID NO: 47):
5′-TAACTTCATGTAAGGCACCGTCAC-3′
BNDG-P2-R (SEQ ID NO: 48):
5′-TCCAGACCTCACCATCAAATGAG-3′

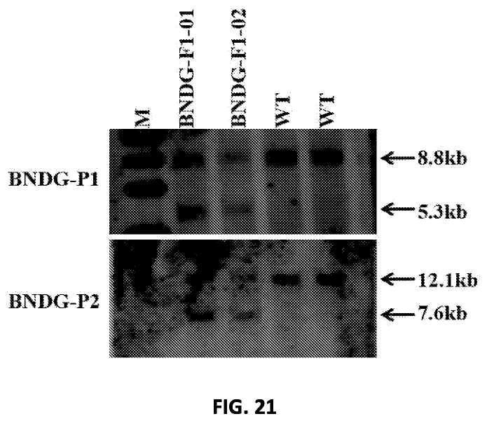

The detection result of Southern Blot is shown in FIG. 21. In view of the hybridization results by BNDG-P1 and BNDG-P2 probes, the two F1 generation mice numbered BNDG-F1-01 and BNDG-F1-02 were confirmed to be positive heterozygotes and no random insertions were detected. This indicates that the method described above can be used to generate genetically-modified MHC gene humanized mice with B-NDG background that can be stably passaged without random insertions.

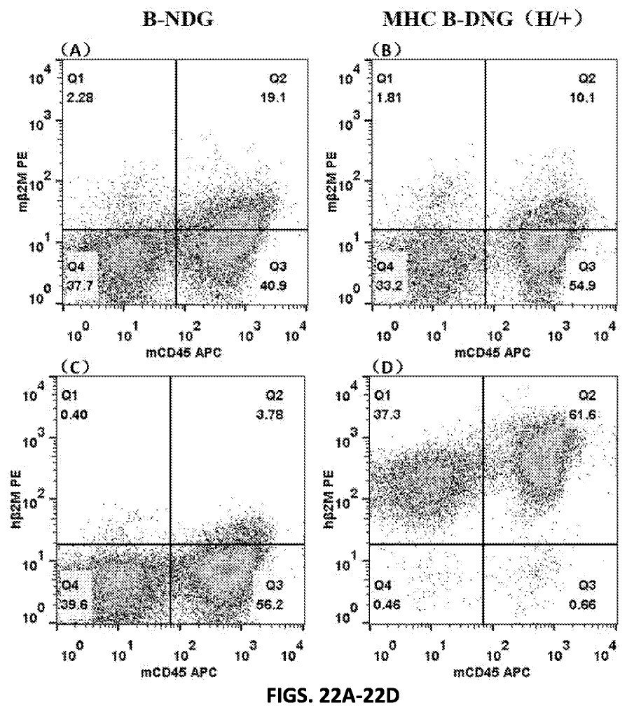

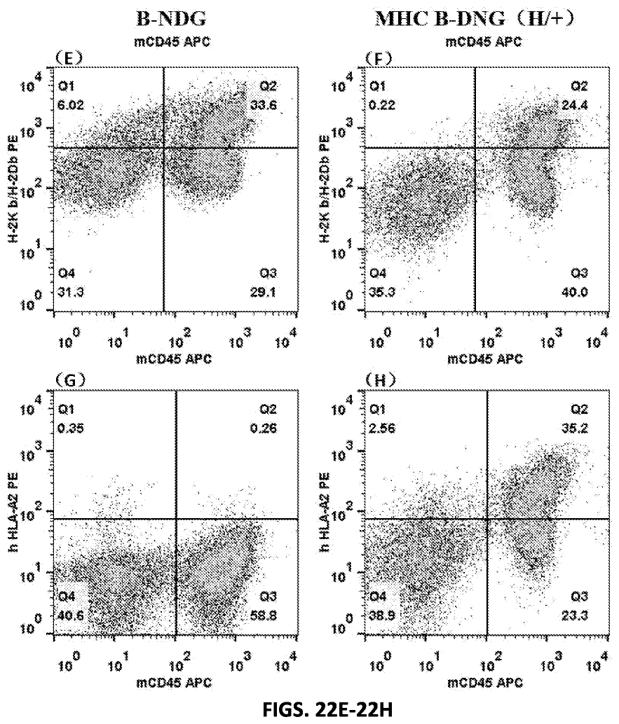

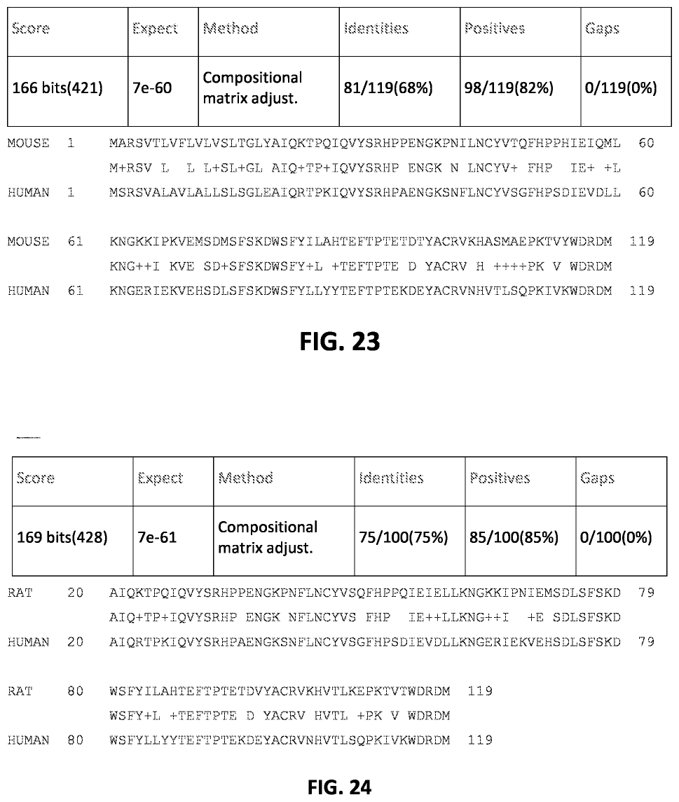

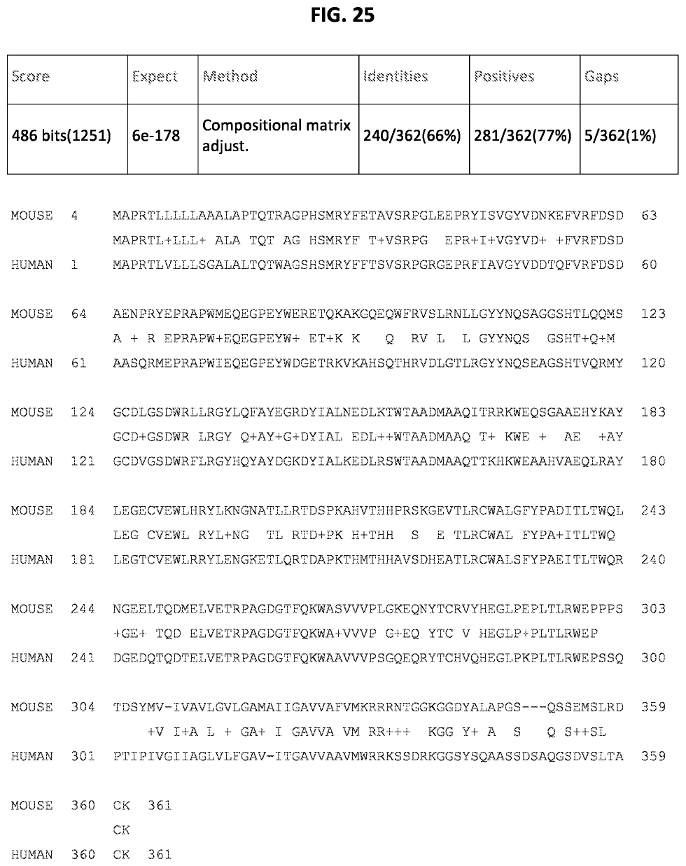

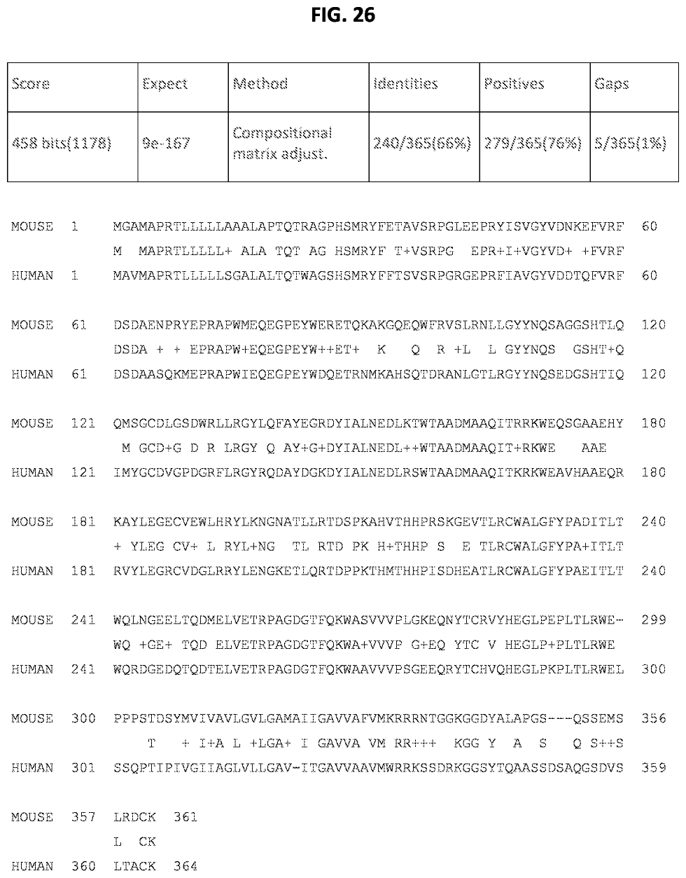

The expression of human B2M protein and HLA-A2.1 protein in positive mice (with B-NDG background) was confirmed by flow cytometry. Specifically, one B-NDG female mouse (6-week old) and one MHC humanized heterozygous female mouse (6-week old) prepared by the method described herein were sacrificed and then spleen cells were collected. Anti-mouse B2M antibody mβ2M PE, anti-mouse H-2Kb/H-2Db antibody, anti-human B2M antibody hβ2M PE, or anti-human HLA-A2 antibody hHLA-A2 PE; together with anti-mouse CD45 antibody mCD45 APC were used for spleen cell staining. The stained cells were subjected to flow cytometry analysis with results shown in FIGS. 22A-22H. The results showed that in B-NDG mice and B-NDG background MHC humanized heterozygous mice, cells expressing mouse B2M (FIGS. 22A and 22B) and cells expressing mouse H2-D1 (FIGS. 22E and 22F) were detected. However, cells expressing human B2M protein (FIG. 22D) and cells expressing human HLA-A2 protein (FIG. 22H) were only detected in humanized MHC mice with B-NDG background. Cells expressing human B2M protein (FIG. 22C) and cells expressing human HLA-A2 protein (FIG. 22G) were not detected in B-NDG mice.

### Example 3. Method Based on Embryonic Stem Cells

The non-human mammals can also be prepared through other gene editing systems and approaches, which includes, but is not limited to, gene homologous recombination techniques based on embryonic stem cells (ES), zinc finger nuclease (ZFN) techniques, transcriptional activator-like effector factor nuclease (TALEN) technique, homing endonuclease (megakable base ribozyme), or other molecular biology techniques. In this example, the conventional ES cell gene homologous recombination technique is used as an example to describe how to obtain a MHC humanized mouse by other methods.

According to the gene editing strategy of the methods described herein and the modified B2M gene locus in MHC humanized mice (FIGS. 8-9), a targeting strategy can be designed with different targeting vector. In view of the fact that one of the objects is to disrupt the coding region of the mouse B2M gene coding region, and to knock in a nucleic acid sequence encoding human B2M protein, a portion of human HLA-A2.1 protein, and a portion of mouse H2-D1 protein at the mouse B2M gene locus, a targeting vector that contains a 5′ homologous arm, a 3′ homologous arm, and a humanized gene fragment is designed. The vector can also contain a resistance gene for positive clone screening, such as neomycin phosphotransferase coding sequence Neo. On both sides of the resistance gene, two site-specific recombination systems in the same orientation, such as Frt or LoxP, can be added. Furthermore, a coding gene with a negative screening marker, such as the diphtheria toxin A subunit coding gene (DTA), can be constructed downstream of the recombinant vector 3′ homologous arm. Vector construction can be carried out using methods known in the art, such as restriction enzyme digestion and ligation. The recombinant vector with correct sequence can then be transfected into mouse embryonic stem cells, and then the recombinant vector can be screened by the positive clone screening gene. The cells transfected with the recombinant vector are next screened by using the positive clone marker gene, and Southern Blot can be used for DNA recombination identification. For the selected correct positive clones, the positive clonal cells (black mice) are injected into the isolated blastocysts (white mice) by microinjection according to the method described in the book A. Nagy, et al., “Manipulating the Mouse Embryo: A Laboratory Manual (Third Edition),” Cold Spring Harbor Laboratory Press, 2003. The resulting chimeric blastocysts formed following the injection are transferred to the culture medium for a short time culture and then transplanted into the fallopian tubes of the recipient mice (white mice) to produce F0 generation chimeric mice (black and white). The F0 generation chimeric mice with correct gene recombination are then selected by extracting the mouse tail genomic DNA and PCR analysis for subsequent breeding and identification. The F1 generation mice are obtained by mating the F0 generation chimeric mice with wild-type mice. By extracting tail genomic DNA and PCR analysis, positive F1 generation heterozygous mice that can be stably passed are selected. Next, the F1 heterozygous mice are bred to each other to obtain genetically recombinant positive F2 generation homozygous mice. In addition, the F1 heterozygous mice can also be bred with Flp or Cre mice to remove the positive clone screening marker gene (Neo, etc.), and then the humanized homozygous mice can be obtained by breeding these mice with each other. The methods of genotyping and phenotypic detection of the obtained F1 heterozygous mice or F2 homozygous mice are similar to those used in the examples described above.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified non-human animal expressing a fusion protein comprising β2 microglobulin (B2M) and a human or humanized major histocompatibility complex (MHC) α chain.

2. The animal of claim 1, wherein the genome of the animal comprises at least one chromosome comprising a sequence encoding the fusion protein.

3. The animal of claim 1 or 2, wherein the fusion protein comprises a human or humanized B2M protein.

4. The animal of any one of claims 1-3, wherein the MHC α chain is a MHC class I α chain.

5. The animal of any one of claims 1-4, wherein the MHC α chain is a human HLA-A protein.

6. The animal of any one of claims 1-4, wherein the MHC α chain is a chimeric MHC α chain.

7. The animal of any one of claims 1-4, wherein the MHC α chain is a human HLA-A/mouse H2-D1 chimeric molecule.

8. The animal of claim 1, wherein the fusion protein comprises a human B2M protein and a chimeric MHC α chain comprising human HLA-A α1 and α2 domains.

9. The animal of claim 8, wherein the chimeric MHC α chain further comprises a mouse H2-D1 α3 domain.

10. The animal of claim 1, wherein the fusion protein comprises a human B2M protein and a human HLA-A protein.

11. The animal of any one of claims 2-10, wherein the sequence encoding the fusion protein is operably linked to an endogenous regulatory element (e.g., a promoter) at the endogenous β2 microglobulin (B2M) gene locus in the at least one chromosome.

12. The animal of any one of claims 2-10, wherein the sequence encoding the fusion protein is operably linked to an endogenous regulatory element (e.g., a promoter) at the endogenous MHC gene locus in the at least one chromosome.

13. The animal of any one of claims 2-10, wherein the animal is a mouse, and the sequence encoding the fusion protein is operably linked to an endogenous regulatory element at the mouse H2-D1 gene locus in the at least one chromosome.

14. The animal of any one of claims 5-13, wherein the human HLA-A is human HLA-A2.1.

15. The animal of any one of claims 5-13, wherein the human HLA-A is human HLA-A1*0101.

16. The animal of any one of claims 1-4, wherein the fusion protein comprises
(a) a human B2M; and
(b) a human HLA-A.

17. The animal of claim 16, wherein the human B2M and the human HLA-A are linked via a linker peptide sequence.

18. The animal of claim 16 or 17, wherein the human B2M comprises or consists of an amino acid sequence that is at least 70%, 75%, 80%, 85%, 90%, 95%, 99%, or 100% identical to SEQ ID NO: 4 or amino acids 21-119 of SEQ ID NO: 4.

19. The animal of any one of claims 16-18, wherein the human HLA-A is HLA-A2.1.

20. The animal of any one of claims 16-19, wherein the human HLA-A comprises or consists of an amino acid sequence that is at least 70%, 75%, 80%, 85%, 90%, 95%, 99%, or 100% identical to SEQ ID NO: 8, amino acids 25-365 of SEQ ID NO: 8, SEQ ID NO: 59, or amino acids 22-362 of SEQ ID NO: 59.

21. The animal of any one of claims 16-20, wherein the fusion protein comprises or consists of an amino acid sequence that is at least 70%, 75%, 80%, 85%, 90%, 95%, 99%, or 100% identical to SEQ ID NO: 62.

22. The animal of any one of claims 1-4, wherein the fusion protein comprises
(a) a human B2M; and
(b) a chimeric MHC α chain.

23. The animal of claim 22, wherein the human B2M and the chimeric MHC α chain are linked via a linker peptide sequence.

24. The animal of claim 22 or 23, wherein the human B2M comprises or consists of an amino acid sequence that is at least 70%, 75%, 80%, 85%, 90%, 95%, 99%, or 100% identical to SEQ ID NO: 4 or amino acids 21-119 of SEQ ID NO: 4.

25. The animal of any one of claims 22-24, wherein the chimeric MHC α chain comprises human HLA-A α1 and α2 domains.

26. The animal of claim 25, wherein the chimeric MHC α chain further comprises a human HLA-A α3 domain.

27. The animal of claim 25, wherein the chimeric MHC α chain further comprises an endogenous MHC α3 domain and/or an endogenous MHC cytoplasmic region.

28. The animal of any one of claims 22-27, wherein the chimeric MHC α chain comprises an amino acid sequence that is at least 70%, 75%, 80%, 85%, 90%, 95%, 99%, or 100% identical to SEQ ID NO: 8, amino acids 25-206 of SEQ ID NO: 8, SEQ ID NO: 59, or amino acids 22-203 of SEQ ID NO: 59.

29. The animal of any one of claims 22-28, wherein the chimeric MHC α chain comprises a α3 domain, a connecting peptide, a transmembrane region, and a cytoplasmic region of an endogenous MHC.

30. The animal of any one of claims 22-28, wherein the animal is a mouse, and the chimeric MHC α chain comprises a α3 domain, a connecting peptide, a transmembrane region, and a cytoplasmic region of mouse H2-D1.

31. The animal of any one of claims 22-30, wherein the chimeric MHC α chain comprises an amino acid sequence that is at least 70%, 75%, 80%, 85%, 90%, 95%, 99%, or 100% identical to amino acids 207-362 of SEQ ID NO: 6.

32. The animal of any one of claims 22-31, wherein the chimeric MHC α chain comprises an amino acid sequence that is at least 70%, 75%, 80%, 85%, 90%, 95%, 99%, or 100% identical to SEQ ID NO: 61 or SEQ ID NO: 63.

33. The animal of any one of claims 1-32, wherein the fusion protein further comprises a signal peptide of human HLA-A2.1 (e.g., at the N-terminus of the fusion protein).

34. The animal of claim 33, wherein the signal peptide comprises an amino acid sequence that is at least 70%, 75%, 80%, 85%, 90%, 95%, 99%, or 100% identical to amino acids 1-21 of SEQ ID NO: 59.

35. The animal of any one of claims 1-34, wherein the animal is heterozygous with respect to the sequence encoding the fusion protein.

36. The animal of any one of claims 1-34, wherein the animal is homozygous with respect to the sequence encoding the fusion protein.

37. A genetically-modified, non-human animal whose genome comprises at least one chromosome comprising a sequence encoding a chimeric MHC α chain comprising a human HLA-A α1 domain, a human HLA-A α2 domain and an endogenous MHC α3 domain.

38. The animal of claim 37, wherein the sequence encoding the chimeric MHC α chain is operably linked to an endogenous regulatory element at the endogenous MHC α chain gene locus in the at least one chromosome.

39. The animal of claim 37 or 38, wherein the genome of the animal further comprises a sequence encoding a human B2M, wherein the human B2M and the chimeric MHC α chain can associate with each other, forming a functional MHC protein complex in the animal.

40. The animal of claim 39, wherein the sequence encoding the human B2M is operably linked to an endogenous regulatory element (e.g., a promoter) at the endogenous B2M gene locus.

41. The animal of any one of claims 37-40, wherein the animal is a mouse, and the sequence encoding the chimeric MHC α chain is operably linked to an endogenous regulatory element (e.g., a promoter) at the mouse H2-D1 gene locus.

42. The animal of any one of claims 37-41, wherein the human HLA-A is human HLA-A2.1.

43. A genetically-modified, non-human animal whose genome comprises at least one chromosome comprising a sequence encoding a human HLA-A.

44. The animal of claim 43, wherein the sequence encoding the human HLA-A is operably linked to an endogenous regulatory element at the endogenous MHC α chain gene locus in the at least one chromosome.

45. The animal of claim 43 or 44, wherein the genome of the animal further comprises a sequence encoding a human B2M, wherein the human B2M and the human HLA-A can associate with each other, forming a functional MHC protein complex in the animal.

46. The animal of claim 45, wherein the sequence encoding the human B2M is operably linked to an endogenous regulatory element (e.g., a promoter) at the endogenous B2M gene locus.

47. The animal of any one of claims 43-46, wherein the animal is a mouse, and the sequence encoding the human HLA-A is operably linked to an endogenous regulatory element (e.g., a promoter) at the mouse H2-D1 gene locus.

48. The animal of any one of claims 43-47, wherein the human HLA-A is human HLA-A2.1.

49. The animal of any one of claims 1-48, wherein the animal does not express endogenous B2M.

50. The animal of any one of claims 1-49, wherein the animal does not express endogenous MHC α chain.

51. The animal of any one of claims 1-50, wherein B2M and the MHC α chain can associate with each other, forming a functional MHC protein complex, wherein the protein complex can present a non-self antigen to the surface of one or more cells.

52. The animal of claim 51, wherein a human T cell (e.g., a cytotoxic T cell) can recognize the presented non-self antigen and initiate immune response.

53. The animal of claim 51, wherein an endogenous T cells (e.g., a cytotoxic T cell) can recognize the presented non-self antigen and initiate immune response.

54. The animal of any one claims 1-53, wherein the animal is a mammal, e.g., a monkey, a rodent or a mouse.

55. The animal of claim 54, wherein the animal is a mouse (e.g., with a C57BL/6 background).

56. The animal of any one of claims 1-55, wherein the animal is an immunodeficient mouse.

57. The animal of any one of claims 1-56, wherein the genome of the animal comprises a disruption in the animal's endogenous CD132 gene.

58. The animal of any one of claims 1-57, wherein the animal is a NOD/scid mouse, a NOD/scid nude mouse, or a B-NDG mouse.

59. The animal of any one of claims 1-58, wherein the animal further comprises a sequence encoding an additional human or chimeric protein.

60. The animal of claim 59, wherein the additional human or chimeric protein is programmed cell death protein 1 (PD-1), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, SIRPα, CD47, THPO, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α(SIRPα) or TNF Receptor Superfamily Member 4 (OX40).

61. A method for making a genetically-modified, non-human animal, comprising: replacing in at least one cell of the animal, at an endogenous B2M gene locus, a sequence encoding a region of endogenous B2M with a sequence encoding a human B2M or a sequence encoding a fusion protein comprising a human B2M and a human or humanized MHC α chain.

62. The method of claim 61, wherein the sequence encoding the region of endogenous B2M comprises all or a part of exon 1, exon 2, and exon 3 of endogenous B2M gene.

63. A method for making a genetically-modified, non-human animal, comprising: replacing in at least one cell of the animal, at an endogenous MHC gene locus, a sequence encoding a region of endogenous MHC α chain with a sequence encoding a human MHC α chain or a sequence encoding a fusion protein comprising a human B2M and a human or humanized MHC α chain.

64. The method of claim 63, wherein the sequence encoding the region of endogenous MHC molecule comprises all or a part of exon 1, exon 2, exon 3, exon 4, exon 5, exon 6, exon 7, and exon 8 of endogenous MHC gene.

65. The method of claim 63, wherein the animal is mouse, and the sequence encoding the region of endogenous MHC comprises all or a part of exon 1, exon 2, exon 3 of mouse H2-D1 gene.

66. The method of any one of claims 61-65, wherein the sequence encoding the fusion protein comprises the following elements:
(a) exon 1, exon 2, and/or exon 3 of human B2M;
(b) an optional sequence encoding a linker peptide sequence; and
(c) exon 2 and/or exon 3 of human HLA-A2.1.

67. The method of claim 66, wherein the sequence encoding the fusion protein further comprises exon 4, exon 5, exon 6, exon 7, and/or exon 8 of the endogenous MHC molecule gene that is downstream of element (c).

68. The method of claim 66 or 67, wherein the animal is mouse, and the sequence encoding the fusion protein further comprises the 3′ UTR of mouse H2-D1 gene.

69. The method of any one of claims 61-68, wherein the fusion protein comprises an amino acid sequence that is at least 70%, 75%, 80%, 85%, 90%, 95%, 99%, or 100% identical to SEQ ID NO: 61, SEQ ID NO: 62, SEQ ID NO: 63, or SEQ ID NO: 64.

70. A method of determining effectiveness of an agent or a combination of agents for the treatment of cancer, comprising:
engrafting tumor cells to the animal of any one of claims 1-60, thereby forming one or more tumors in the animal;
administering the agent or the combination of agents to the animal; and
determining the inhibitory effects on the tumors.

71. The method of claim 70, wherein before engrafting the tumor cells to the animal, human peripheral blood cells (hPBMC) or human hematopoietic stem cells are injected to the animal.

72. The method of claim 70, wherein the tumor cells are from cancer cell lines.

73. The method of claim 70, wherein the tumor cells are from a tumor sample obtained from a human patient.

74. The method of claim 70, wherein the inhibitory effects are determined by measuring the tumor volume in the animal.

75. The method of claim 70, wherein the tumor cells are melanoma cells, lung cancer cells, primary lung carcinoma cells, non-small cell lung carcinoma (NSCLC) cells, small cell lung cancer (SCLC) cells, primary gastric carcinoma cells, bladder cancer cells, breast cancer cells, and/or prostate cancer cells.

76. A method of producing an animal comprising a human hemato-lymphoid system, the method comprising:
engrafting a population of cells comprising human hematopoietic cells or human peripheral blood cells into the animal of any one of claims 1-60.

77. The method of claim 76, wherein the human hemato-lymphoid system comprises human cells selected from the group consisting of hematopoietic stem cells, myeloid precursor cells, myeloid cells, dendritic cells, monocytes, granulocytes, neutrophils, mast cells, lymphocytes, and platelets.

78. The method of claim 76 or 77, further comprising: irradiating the animal prior to the engrafting.

79. A fusion protein comprising β2 microglobulin (B2M) and a human or humanized MHC α chain.

80. A nucleic acid encoding the fusion protein of claim 79.

81. A protein comprising an amino acid sequence, wherein the amino acid sequence is one of the following:
(f) an amino acid sequence set forth in SEQ ID NO: 4, 8, 59, 61, 62, 63, or 64;
(g) an amino acid sequence that is at least 90% identical to SEQ ID NO: 4, 8, 59, 61, 62, 63, or 64;
(h) an amino acid sequence that is at least 91%, 92%, 93%, 94%, 95%, 96%, 97%, 98%, or 99% identical to SEQ ID NO: 4, 8, 59, 61, 62, 63, or 64;
(i) an amino acid sequence that is different from the amino acid sequence set forth in SEQ ID NO: 4, 8, 59, 61, 62, 63, or 64 by no more than 10, 9, 8, 7, 6, 5, 4, 3, 2 or 1 amino acid; and
(j) an amino acid sequence that comprises a substitution, a deletion and/or insertion of one, two, three, four, five or more amino acids to the amino acid sequence set forth in SEQ ID NO: 4, 8, 59, 61, 62, 63, or 64.

82. A nucleic acid comprising a nucleotide sequence, wherein the nucleotide sequence is one of the following:
(e) a sequence that encodes the protein of claim 81;
(f) SEQ ID NO: 9, 10, 13, 14, 15, 16, 52, 54, or 65;
(g) a sequence that is at least 90% identical to SEQ ID NO: 9, 10, 13, 14, 15, 16, 52, 54, or 65; and
(h) a sequence that is at least 91%, 92%, 93%, 94%, 95%, 96%, 97%, 98%, or 99% identical to SEQ ID NO: 9, 10, 13, 14, 15, 16, 52, 54, or 65.

83. A cell comprising the protein of claim 81 and/or the nucleic acid of claim 82.

84. An animal comprising the protein of claim 81 and/or the nucleic acid of claim 82.
